(19) United States
(12) Patent Application Publication
(10) Pub. No.: US 2024/0309360 A1
(43) Pub. Date: Sep. 19, 2024

(54) TRANS-ACTIVATORS AND METHODS AND USE THEREOF

(71) Applicant: THE GOVERNING COUNCIL OF THE UNIVERSITY OF TORONTO, Toronto (CA)

(72) Inventors: Mikko Joonas Oskari Taipale, Ontario (CA); Nader Alerasool, San Francisco, CA (US); He Leng, Toronto (CA)

(21) Appl. No.: 18/575,279

(22) PCT Filed: Jul. 14, 2022
(86) PCT No.: PCT/CA2022/051100

§ 371 Date: Dec. 28, 2023

Related U.S. Application Data
(60) Provisional application No. 63/221,611, filed on Jul. 14, 2021.

(51) Int. Cl. C12N 15/10; C12N 9/22; C12N 15/11; C12N 15/90

(52) U.S. Cl. CPC C12N 15/1082; C12N 9/22; C12N 15/11; C12N 15/907; C12N 2310/20; C12N 2800/80

(57) ABSTRACT

A heterologous transcriptional activator comprising a DNA targeting domain, preferably a catalytically inactive DNA targeting protein such as a CRISPR-Cas protein, and an effector domain comprising at least one transactivation domain described herein or an functional variant thereof. Also provided herein are expression constructs, vectors, and cells encoding or expressing said transcriptional activator, as well as systems and methods for transcriptional activation of a target gene, and compositions, kits and reagents employed in the making and use thereof.

### CROSS-REFERENCE TO RELATED APPLICATIONS

The present disclosure claims the benefit of priority from U.S. patent application No. 63/221,611, filed on Jul. 14, 2021, the contents of which is incorporated herein by reference in its entirety.

### FIELD

The present disclosure relates to reagents and methods for transcriptional activation and in particular to the use of heterologous transactivator domains in transcriptional activators for targeted transcriptional activation.

### INTRODUCTION

Transcription of protein-coding genes is orchestrated by a coordinated interplay of transcription factors (TFs) that bind DNA in a sequence-specific manner, RNA polymerase II machinery that initiates transcription from promoters, and diverse chromatin-associated factors and complexes that modulate chromatin structure and act as bridges between TFs and RNA pol II (Cramer, 2019). The human genome encodes thousands of proteins that are involved in various stages of transcriptional regulation, and the ready availability of methods such as ChIP-seq has revealed the genomic binding sites of hundreds of factors in diverse conditions (The ENCODE Project Consortium et al., 2020). At the same time, systematic studies have characterized or inferred the DNA-binding specificity of about three-quarters of human TFs (Badis et al., 2009; Jolma et al., 2013; Lambert et al., 2018; Najafabadi et al., 2015; Weirauch et al., 2014). Similarly, interaction proteomics approaches have uncovered many chromatin-associated proteins and characterized the composition of transcriptional regulatory complexes in human cells (Gao et al., 2012; Huttlin et al., 2020; Lambert et al., 2019; Li et al., 2015; Marcon et al., 2014; Mashtalir et al., 2018).

However, whether and how TFs and chromatin-associated factors promote transcriptional activation or repression (or regulate chromatin states by other means) has remained largely unknown due to the limited causal insights afforded by these methods. For example, the vast majority of genomic binding sites observed in ChIP-seq experiments are not causally associated with transcriptional events. That is, knockdown or knockout of a given transcriptional regulator does not affect the transcription of most genes that the regulator binds to. On the other hand, sequence-based annotation of transcriptional regulators has been challenging, because most transcriptional effector functions are encoded by degenerate linear motifs rather than folded and conserved protein domains (Arnold et al., 2018; Erijman et al., 2020; Sigler, 1988; Staller et al., 2021).

Artificial recruitment, also known as activator bypass, is powerful method to characterize the transcriptional effect of diverse proteins in a defined context (Ptashne and Gann, 1997; Sadowski et al., 1988). In this approach, proteins or their fragments are ectopically recruited to a reporter gene by fusing the protein to the DNA-binding domain of a well-characterized TF such as Gal4 or TetR. The defined context alleviates the challenges posed by endogenous gene regulation, where multiple factors bind regulatory elements in concert, hindering causal inference. Artificial recruitment has been traditionally used to identify transcriptional activators or transactivation domains (TADs) in individual transcriptional regulators (Ptashne and Gann, 1997). However, recent studies have characterized the transcriptional effects of large collections of regulators in fruit flies or yeast by individually tethering them to reporter genes (Keung et al., 2014; Stampfel et al., 2015). Due to the limited scalability of the arrayed format, these studies focused on known regulators rather than potentially novel factors. Moreover, classical model organisms lack several regulatory mechanisms and layers critical for gene expression in mammals, such as enhancers (yeast) or DNA methylation (yeast and fruit flies). More recently, Tycko and colleagues implemented an unbiased pooled screening strategy to characterize the transcriptional activation potential of annotated human protein domains (Tycko et al., 2020). This study highlighted the value of unbiased approaches in identifying novel transcriptional regulators, such as the unexpected role of variant KRAB domains in transcriptional activation instead of repression. Yet, because most transcriptional activation domains are encoded by disordered regions (Arnold et al., 2018; Dyson and Wright, 2005), it is likely that a domain-focused screen misses a significant fraction of transactivators.

Thus, despite the increased knowledge of the composition of transcriptional regulator complexes and their genomic binding patterns, a complete understanding of the downstream effects elicited by TFs and diverse chromatin-associated factors is lacking.

### SUMMARY

Described herein, the inventors have established a platform to systematically identify and characterize the transcriptional regulatory potential of human proteins in an unbiased manner. By screening over 13,000 proteins in a pooled format, several hundred potent activators were identified, many of which were previously poorly annotated. Transactivation domains were systematically uncovered among the hits, including some that do not adhere to the canonical “acidic blob” model of activation domains. Furthermore, interaction proteomics were combined with chemical inhibitors to delineate the co-factor specificity of both novel and known transcriptional activators, highlighting how even highly related TFs with virtually identical DNA-binding specificities can activate transcription through distinct co-factor complexes.

The inventors describe herein the first systematic screen of transcriptional activators in human cells. Several hundred transcriptional activators were identified that were, as expected, enriched in sequence-specific transcription factors and other chromatin-associated proteins.

The results shown herein suggest that only a very limited number of TFs are strong transcriptional activators. This makes sense in the context of the known DNA-binding specificities and chromatin occupancies of human TFs (Jolma et al., 2013; The ENCODE Project Consortium et al., 2020; Yan et al., 2013). Most TFs recognize short (˜6-10 bp) motifs and associate with thousands of sites across the genome. Yet, most binding events are not associated with transcriptional output. Strong transcriptional activation domains coupled to limited sequence specificity of the DNA-binding domain would lead to spurious activation of large swaths of genes, likely impeding cellular fitness. Many of the strongest activators identified herein were not DNA-binding transcription factors but other chromatin-associated proteins.

The discovery of novel and highly potent human transactivation domains has also therapeutic implications. Components derived from viral proteins, such as the VP16 transactivation domain or the tripartite VPR activator, can elicit immune responses in vivo and lead to adverse effects in the clinic. Designing synthetic transcriptional regulators from fully human components is expected to be advantageous in therapeutic applications (Israni et al., 2021). Furthermore, also shown herein, combining activation domains from multiple different human proteins can generate “superactivators” that are able to robustly upregulate genes even in highly compacted regions of the genome.

An aspect includes a heterologous transcriptional activator comprising:
a DNA targeting domain, optionally an enzymatically inactive CRISPR-CAS protein, a zinc finger DNA binding domain, a tet-repressor or transcriptional activator-like effector (TALE) DNA binding domain; and
an effector domain comprising:
   at least one transactivation domain (TAD) selected from the TADs listed in any one of Tables 1 to 6, optionally Table 2 or Table 6, or a functional variant thereof, or
   at least two TADs selected from the TADs listed in any one of Tables 1-6, optionally Table 1 or Table 3, or functional variants of any thereof, preferably at least one TAD selected from the TADs listed in Table 4 or Table 5 or Table 6, or functional variants thereof,
wherein the DNA targeting domain and effector domain are operably linked.

An aspect includes an isolated nucleic acid encoding an effector domain described herein

An aspect includes an isolated nucleic acid encoding a heterologous transcriptional activator described herein.

An aspect includes an expression construct comprising a nucleic acid described herein operably linked to one or more promoters and one or more transcription termination sites.

An aspect includes a vector comprising a nucleic acid or expression construct described herein, optionally wherein the vector is an adenoviral or lentiviral vector.

An aspect includes a cell comprising a transcriptional activator, nucleic acid, expression construct, or vector described herein.

An aspect includes a transcriptional activation system comprising: a heterologous transcriptional activator described herein, wherein the DNA targeting domain comprises a CRISPR-Cas protein and at least one gRNA.

An aspect includes a method of activating transcription of a target gene in a cell, the method comprising: a) introducing into the cell a transcriptional activator, nucleic acid, expression construct, or vector described herein; and b) culturing the cell under suitable conditions such that the effector domain activates transcription of the target gene.

An aspect includes a screening method, the method comprising: a) introducing into a plurality of cells a transcriptional activator, one or more nucleic acids, one or more expression constructs, or one or more vectors described herein, wherein the DNA targeting domain comprises a CRISPR-Cas protein; and a plurality of gRNAs; or introducing a plurality of gRNAs into a population of cells described herein wherein the DNA targeting domain comprises a CRISPR-Cas protein; b) culturing the plurality of cells such that the one or more gRNAs associate with the CRISPR-Cas protein and guides the transcriptional activator to a CRISPR target site such that the effector domain activates transcription of a target gene; c) optionally treating with an amount of a test drug or toxin; d) optionally culturing the plurality of cells for a period of time to allow for gRNA dropout or enrichment; and e) collecting the plurality of cells, or a subset thereof.

An aspect includes a composition comprising a transcriptional activator, nucleic acid, expression construct, vector, or cell described herein.

An aspect includes a kit comprising a vial and a heterologous transcriptional activator, nucleic acid, expression construct, vector, cell, or composition described herein and optionally one or more of: an inducing agent, a gRNA or a gRNA expression construct.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 depicts the pooled ORFeome screen for transcriptional activators. A, shows a schematic of the chemically-induced dimerization system to characterize transcriptional activators in human cells. B, shows the percent of high GFP cells when the indicated constructs were transfected into HEK293T reporter cells and treated with abscisic acid or DMSO for 48 hours. C, Outline of the pooled ORFeome screen for transcriptional activators. D, Enrichment of high GFP cells in the pooled ORFeome screen after 48 hour ABA treatment. E, Enrichment of ORFs in the high GFP pool compared to the unsorted ORFeome. F, Enrichment of Gene Ontology categories among positive screen hits. G, Enrichment of InterPro domains among positive screen hits. H, Enrichment of CORUM complexes among screen hits.

FIG. 2 shows transcriptional activity of transcription factor families. Transcriptional regulators were individually tested for activation of the reporter in an arrayed manner. DNA-binding specificity (shown as sequence logos) is from CisBP (Weirauch et al., 2014). Asterisks indicate statistically significant activators (FDR <0.05). A, Homeobox family proteins B, Forkhead box proteins, C, Kruppel-like factors, D, SRY-related HMG-box (SOX) proteins, E, Polycomb-group RING Finger (PCGF) proteins. Composition of canonical (cPRC1) and non-canonical (ncPRC1) complexes is shown on the right. F, Spy1/RINGO family proteins. Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006).

FIG. 3 shows systematic discovery of transactivation domains in human proteins with TAD-seq. A, Outline of the TAD-seq pooled assay. B, Examples of known transactivation domains. Domain organization is shown on top. TAD-seq plot shows the fold enrichment of RNAseq reads in the high GFP population or the medium GFP population. Each circle shows the mid-point (30th amino acid) of the 60-aa tile. Filled circles indicate statistically significant hits. Grey boxes indicate previously described transactivation domains. C, Examples of novel transactivation domains. Labeling is as in panel B. D, Location of the transactivation domain of HOXA2. E, Sequences of the activating fragments identified in HOXA2. Activating fragments are in bold. The region common to all three fragments that were enriched in the medium GFP population is indicated as overlap of activating fragments. The location of the antennapedia-like hexapeptide sequence is indicated as hexapeptide. F, Location of the transactivation domain in YAF2. G, Crystal structures of RING1B RAWUL domain bound to the YAF2_RYBP domain of RYBP (PDB 31XS) and the CBX_C domain of CBX7 (PDB 3GS2). H, YAF2_RYBP domains and CBX_C domains from indicated proteins were individually tested for transcriptional activity. Asterisks indicate statistically significant activators (FDR <0.05). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006). Right, in vitro affinity of the domains towards RING1B (Wang et al., 2008, 2010). Statistical significance was calculated with an unpaired two-tailed t-test.

FIG. 4. shows co-factor specificity of transcriptional activators. A, Proximity partners of indicated transcriptional regulators were identified with BiolD2. Enrichment of selected co-factor complexes is shown as a heat map. Average spectral counts (n+1) were normalized to background spectral counts (n+1) of EGFP and Nanoluc baits. B, Interaction patterns of activating Forkhead transcription factors based on the AP-MS study of (Li et al., 2015). Spectral counts were normalized as in panel A. C, Left, effect of p300/CBP inhibition by A-485 on the activity of 83 transcriptional regulators. Known p300 interactors are shown as solid dark circles, known NuA4 interactors as outlined circles. Right, p300 interactors are significantly more affected than other transcriptional regulators by A-485 treatment. Statistical significance was calculated with one-way ANOVA using Dunnett's multiple testing correction. D, Left, effect of BET bromodomain protein inhibition by JQ1 on the activity of 83 transcriptional regulators. Known p300 interactors are shown as solid dark circles, known NuA4 interactors as outlined circles. Right, p300 interactors are significantly more affected than other transcriptional regulators by A-485 treatment. Statistical significance was calculated with one-way ANOVA using Dunnett's multiple testing correction. E, Clustering of transcriptional activators based on their sensitivity on diverse inhibitors. Clustering was performed with Euclidian distance with average linkage. Clusters enriched in known CBP/p300 interactors (solid circles) and NuA4 interactors crosses are indicated.

FIG. 5 shows SRF-C3orf62 fusion generates a potent p300-dependent transcriptional activator that promotes expression of SRF/MRTF target genes. A, Schematic of the JAZF1-SUZ12 fusion found in low-grade endometrial stromal sarcomas. The transactivation domain identified by TAD-seq is indicated. B, Schematic of the SRF-C3orf62 fusion found in myofibroma/myopericytoma. The transactivation domain identified by TAD-seq is indicated. C, JAZF1, SUZ12 and JAZF1-SUZ12 proximity interactors were identified with BiolD2. JAZF1-SUZ12 interacts with both PRC2 components and NuA4 components, generating a supercomplex. D, SRF, C3orf62, C3orf62-Cterm and SRF-C3orf62 proximity interactors were identified with BiolD. SRF-C3orf62 fusion robustly interacts with CBP and p300. E, SRF-C3orf62 is a potent transcriptional activator. Indicated PYL1 fusions were individually tested for activation of the genomically integrated reporter. F, SRF-C3orf62 activates expression of serum response element reporter in the absence of cofactors. Indicated constructs C-terminally tagged with 3×FLAG-V5 were co-transfected into NIH3T3 cells with an SRE-Firefly luciferase reporter and a constitutive Nanoluc reporter. Relative luciferase activities were measured to assess the activity of each construct. G, NIH3T3 cells stably expressing doxycycline-inducible SRF-C3orf62-GFP or Nanoluc-GFP were treated with doxycycline for 46 hours, of which the last 22 hours in low-serum conditions (0.5% FCS). Gene expression patterns were analyzed by RNA-seq. Significantly upregulated (dark circles, top) and downregulated (medium circles, bottom) genes (absolute log 2 fold change >1, FDR <0.05). Well characterized targets of SRF/MRTF (top, labeled circles) and SRF/TCF (bottom, labeled circles) are indicated. H, Gene set enrichment analysis of SRF-C3orf62-GFP expressing cells compared to Nanoluc-GFP expressing cells.

FIG. 6 shows ORFeome screen for transcriptional activators. A, Distribution of sequencing reads across the ORFeome in pooled plasmid DNA and in infected cells. B, Distribution of ORF sizes in the plasmid pool and in infected cells. C, Transcriptional activity depends on abscisic acid treatment. ORFeome-PYL1 infected cells were treated with 100 μM ABA and the fraction of high GFP cells measured by flow cytometer over time. D, The effect of ABA concentration on transcriptional activity. Reporter cells transfected with the indicated constructs were treated with increasing amounts of ABA for 48 hours. E, No high GFP population is observed in ABA treated cells not expressing the ORFeome-PYL1 library. F, Enrichment of interaction hubs among the hits of the activation screen. G, Enrichment of yeast two-hybrid autoactivators among the hits of the activation screen. H, Individual validation of transcriptional activators identified in the activation screen. Indicated constructs were transfected into the reporter cell line and high GFP cell fraction was measured by flow cytometry after 48-hour treatment with ABA. Asterisks indicate statistically significant ABA-dependent increase in high GFP population (FDR <5%). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006).

FIG. 7 shows transcriptional activity of transcription factor and chromatin-associated protein families. Transcriptional regulators were individually tested for activation of the reporter in an arrayed manner. DNA-binding specificity (shown as sequence logos) is from CisBP (Weirauch et al., 2014). Asterisks indicate statistically significant activators (FDR <5%). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006). A, ETS family TFs B, Atonal-related bHLH factors, C, Myogenic factors, D, Twist/Hand bHLH factors, E, Casein kinases, F, Mediator components, G, Transcription factors identified in the primary activation screen are enriched for factors that can reprogram human iPS cells (right) or mouse ES cells (left) when ectopically expressed (Ng et al., 2021; Theodorou et al., 2009). Statistical significance was calculated with a Wilcoxon rank sum test (left) or Fisher's exact test (right).

FIG. 8 shows differential activity of transcription factors is not explained by expression levels. A, Schematic of the transcriptional activation assay measuring both reporter gene expression and effector protein expression. B, Transactivation of Kruppel-like factors (left) compared to the expression level of each factor as measured by RFP fluorescence (right). Background RFP intensity is shown as a dashed line. C, Forkhead TF activity. D, Homeodomain TF activity, E, SOX TF activity.

FIG. 9 shows TAD-seq identifies transactivation domains. A, High and medium GFP population was assessed by flow cytometry after recruiting 60aa fragments to the reporter with ABA for 48 hours. High GFP and medium GFP cells were sorted by FACS and ORFs enriched in the pools were identified by next-generation sequencing. B, Enrichment and depletion of amino acids among the identified transactivator fragments compared to inactive fragments in the library. Amino acids shown in bold were statistically significantly enriched or depleted. C, Enrichment of predicted transactivation domains among the active fragments. 9aaTADs were predicted with 9aaTAD prediction tool (https://www.med.muni.cz/9aaTAD/) using “Moderately Stringent Pattern”. Only 100% confident matches were considered in the analysis. ADpred algorithm was described in (Erijman et al., 2020). Statistical significance was calculated with Fisher's exact test. D, Predicted TADs among active fragments are longer than those in inactive fragments. Statistical significance was calculated with two-tailed t-test assuming equal variance. E, Enrichment of fragments in the high GFP pool versus the medium GFP pool. Significant hits are shown as outlined circles. F, Individual validation of transactivating fragments. Indicated TADs were fused to PYL1 and transfected into the reporter cells in an arrayed format. Asterisks indicate statistically significant activators (FDR <5%). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006).

FIG. 10 shows systematic discovery of transactivation domains in human proteins with TAD-seq. A, Examples of known transactivation domains. Domain organization is shown on top. TAD-seq plot shows the fold enrichment of RNAseq reads in the high GFP population (dark circles) or the medium GFP population (light circles). Each circle shows the mid-point (30th amino acid) of the 60-aa tile. Filled circles indicate statistically significant hits. Grey boxes indicate previously described transactivation domains. B, Examples of novel transactivation domains. Labeling is as in panel A.

FIG. 11 shows transactivation domains of SPDYE4 and YAF2. A, Alignment of five Spy1/RINGO family proteins: SPDYE4 (SEQ ID NO: 142); SPDYE1 (SEQ ID NO: 143); SPDYE7P (SEQ ID NO: 144); SPDYE2 (SEQ ID NO: 145); and SPDYC (SEQ ID NO: 146). Four fragments screened by TAD-seq (SPDYE4-2 (SEQ ID NO: 154); SPDYE4-3 (SEQ ID NO: 90); SPDYE4-4 (SEQ ID NO: 91); and SPDYE4-5 (SEQ ID NO: 155)) are shown above the alignment, and active fragments are indicated in bold. Dashed box indicates the inferred minimal activating domain. Note that the region of the minimal activating domain is not conserved in SPDYC, which was the only Spy1/RINGO family member that did not activate transcription. B, Alignment of YAF2_RYBP domains of YAF2 (SEQ ID NO: 147) and RYBP (SEQ ID NO: 148), and CBX_C domains of CBX family proteins (SEQ ID NOs: 149-153). The location of the two beta sheets is indicated with arrows.

FIG. 12 shows interaction networks of transcriptional activators. A, BiolD2 network of transcriptional activators. Bait proteins (e.g. FAM90A1, SPDYE4, SS18L2, SOX7, etc.) are shown as light grey rectangles. BAF complex members, p300/CBP, NuA4 complex members, Mediator components, and TFIID components are indicated. The width of the edges indicates average spectral counts of two replicates. For clarity, two highly connected prey proteins (ZNF518A and ZNF518B) were removed from this visualization B, AP-MS network of transcriptional activators. Labeling as in panel A. For clarity, nine highly connected prey proteins (APEH, ACTC1, ALDH1L1, PSDM4, LSS, FLII, PACSIN2, RPS3A, QPCTL) were removed from this visualization.

FIG. 13 shows the effect of small molecule inhibitors on the transcriptional activity of 83 transcriptional regulators. A, Effect of CDK9 inhibition by flavopiridol. Known p300 interactors are shown as solid dark circles, known NuA4 interactors as outlined circles. B, Effect of Casein kinase 2 inhibition by CX4945. Known p300 interactors are shown as solid dark circles, known NuA4 interactors as outlined circles. C, Effect of DYRK1A/DYRK1B inhibition by AZ191. Known p300 interactors are shown as solid dark circles, known NuA4 interactors as outlined circles. DYRK1A/DYRK1B interactor DCAF7, and DCAF7 interactor NCKISPD are highlighted. D, Effect of p300 inhibition by A-485 on transcriptional regulators characterized by AP-MS and BiolD. Asterisks indicate statistically significant activators (FDR <5%). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006). E, Effect of BET bromodomain inhibition by JQ1 on transcriptional regulators characterized by AP-MS and BiolD. Asterisks indicate statistically significant activators (FDR <5%). Statistical significance was calculated with unpaired two-tailed t-test assuming equal variance, and corrected for multiple hypotheses with False Discovery Rate (FDR) approach of Benjamini, Krieger and Yekutieli (Benjamini et al., 2006).

FIG. 14 shows transcriptional activation by SRF-C3orf62. A, SRF, C3orf62, C3orf62-Cterm and SRF-C3orf62 interactors were characterized with AP-MS. SRF-C3orf62 fusion robustly interacts with CBP and p300. B, Analysis of differentially expressed genes in NIH3T3 cells expressing SRF-GFP, C3orf62-GFP, or SRF-C3orf62-GFP compared to cells expressing Nanoluc-GFP. C, Gene Ontology enrichment analysis of significantly upregulated and downregulated genes in NIH3T3 cells expressing SRF-C3orf62-GFP. D, Overlap of genes significantly upregulatedy by SRF-C3orf62-GFP and target genes of SRF/MRTF or SRF/TCF, published previously (Esnault et al., 2014; Gualdrini et al., 2016). Statistical significance was calculated with a hypergeometric distribution test.

FIG. 15 shows the effect of the minimal activating sequence for each individual component of SPDYE4-CITED1-P65-HSF1 fusion on transcriptional activity of an a EGFP reporter. All components were fused to PYL1 dimerization domain and recruited to the reporter with addition of 1 μM abscisic acid (ABA) for 48 hours. Sequence of each fusion is shown in SEQ ID NOs: 121, 123, 127, 129, 131, 133, and 135. Sequences of each of the individual domains tested are provided in SEQ ID NOs: 47, 90, 101-104, 116, and 118.

FIG. 16 shows examples of when fusing two or more transactivation domains and targeting them to the same reporter. Addition of SPDYE4 activation domain showed a marked improvement to activity of CITED1 domain alone. This is in contrast to SPDYE4's weak activity when tethered to the reporter on its own, suggesting synergistic activity when fused to CITED1 activation domain. All fusion constructs indicate activation domains and not full-length proteins, except for “full length CITED1/2”, as indicated, which were tested to compare activity with their corresponding transactivation domains. P300core is the catalytic domain EP300 (amino acids 1048-1664). Reporter cells were treated with 1 μM abscisic acid (ABA) or same volume of DMSO for 48 hours before collection for flow cytometry. Error bars represent S.D. from four replicates.

FIG. 17 shows activity of tethering different combinations and orientations of activation domains from human SPDYE4, CITED1, p65, and HSF1 proteins to an EGFP reporter (left) or promoter of CD133 gene in HEK293T cells. Recruitment was induced by addition of 1 μM abscisic acid (ABA) for either 24 or 48 hours before cells were collected.

FIG. 18 shows the effect of replacing each part of the multi-component SPDYE4-CITED1-P65-HSF1 (SCPH) activator with different activation domains. SPDYE4 activation domain appeared the most indispensable as replacing it with other individually stronger activation domains disrupted activity of the SPCH activator. On the other hand, replacing the CITED1 component with other strong human transactivation domains from our screen had no deleterious effect on activity and even seemed to improve its potency with ZXDC and C3orf62 activation domains. All constructs were tethered to the EGFP reporter by addition of 1 μM abscisic acid for 48 hours.

FIG. 19 shows the effect of 117 different effector domains comprising different combinations of transactivation domains, or fragments thereof, when used in combination with rTetR or dCas9 based recruitment systems. A, Transcriptional activation using the 117 effector domains in combination with a rTetR DNA targeting domain. B, Transcriptional activation using the 117 effector domains in combination with a dCas9 DNA targeting domain. C, Correlation of transcriptional activation between the rTetR and dCas9 based recruitment systems.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

The terms “nucleic acid”, “oligonucleotide”, “primer” as used herein means two or more covalently linked nucleotides. Unless the context clearly indicates otherwise, the term generally includes, but is not limited to, deoxyribonucleic acid (DNA) and ribonucleic acid (RNA), which may be single-stranded (ss) or double stranded (ds). For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The term “oligonucleotide” as used herein generally refers to nucleic acids up to 200 base pairs in length and may be single-stranded or double-stranded. The sequences provided herein may be DNA sequences or RNA sequences, however it is to be understood that the provided sequences encompass both DNA and RNA, as well as the complementary RNA and DNA sequences, unless the context clearly indicates otherwise. For example, the sequence 5′-GAATCC-3′, is understood to include 5′-GAAUCC-3′, 5′-GGATTC-3′, and 5′GGAUUC-3′.

The term “functional variant” as used herein includes modifications of the polypeptide sequences disclosed herein that perform substantially the same function as the polypeptide molecules disclosed herein in substantially the same way. For example, functional variants may include active fragments of the polypeptides described herein, for example an N- and/or C-terminal truncation which retains transcriptional activation activity and/or co-activator interaction. Functional variants may include variants having one or more substituted amino acids and/or which retain at least a minimal sequence identity to the unmodified or non-variant sequence. For example, the functional variant may comprise substitutions of up to 1, 2, 3, or more amino acids for every ten amino acids. For example, the functional variant may comprise sequences having at least 80%, or at least 90%, or at least 95% sequence identity to the sequences disclosed herein. The functional variant may also comprise conservatively substituted amino acid sequences of the sequences disclosed herein. Substitutional amino acid variants are those in which at least one residue in the sequence has been removed and a different residue inserted in its place. An example of substitutional amino acid variants are conservative amino acid substitutions. Functional variants such as active fragments including minimal fragments which can for example be identified as described herein which retain transcriptional activation activity and/or co-activator interaction can be identified for example using the methods described herein.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

The term “heterologous transcriptional activator” or “transcriptional activator described herein” as used herein means an engineered fusion protein or engineered dimer comprising: an effector domain comprising at least one transactivation domain (TAD) selected from the TADs listed in Table 2 and functional variants thereof, or at least two TADS selected from the TADs listed in Table 1, Table 2, Table 3, Table 4, Table 5, and/or Table 6 and functional variants of any one thereof, operably linked to a DNA targeting domain.

The term “operably linked” as used herein refers to a relationship between two components that allows them to function in an intended manner. For example, a first polypeptide may be operably linked to a second polypeptide by covalent linkage (e.g. as a fusion protein), or through one or more interaction components. Similarly, where a reporter gene is operably linked to a promoter, the promoter actuates expression of the reporter gene.

The transcriptional activator may further comprise one or more interaction components of an interaction system, which provides a functional interaction between the effector domain and/or DNA targeting domain and/or target DNA. The term “interaction component” is used herein to encompass one or more components of an interaction system, which together provide said functional interaction. The term “interaction system” as used herein is intended to encompass interaction components that permit covalent or non-covalent interactions, and/or constitutive or inducible interactions. Such interaction systems may include for example a peptide linker, optionally a protease-sensitive peptide linker; one or more dimer, trimer, or higher order multimerization components such as an interaction domain, optionally inducible dimer, trimer, or multimerization components, optionally an inducible interaction domain; and/or one or more components which can modulate subcellular localization of the transcriptional activator. The interaction system can comprise two or more components.

The DNA targeting domain and the effector domain may be covalently linked, for example as domains of a single polypeptide (e.g. fusion protein), or may be linked by an interaction component such as an interaction domain for example, that interact under certain conditions (e.g. as a dimer). Accordingly, the heterologous transcriptional activator may comprise a single polypeptide, or may comprise a first polypeptide comprising a DNA targeting domain and a first interaction component such as a dimer interaction domain, and a second polypeptide comprising an effector domain and a second interaction component such as a dimer interaction domain, wherein the first and second dimer interaction domain can interact, for example under certain conditions. Higher-order multimerization systems, such as the SunTag system (Tenenbaum et al., 2014), are also contemplated herein.

The interaction between the effector domain and/or DNA targeting domain and/or target DNA can be controlled using a variety of inducible interaction systems. For example, the effector domain and DNA targeting domain may be linked by a protease-sensitive linker such as a self-cleaving NS3 protease domain, which is stabilized in the presence of an NS3 inhibitor such as grazoprevir. In another example, localization of the DNA targeting domain and/or effector domain to the nucleus can be controlled by an interaction component such as a localization domain, for example tamoxifen-regulated nuclear localization using estrogen receptor ligand binding domain variants. In a further example, the DNA targeting domain can be linked to a first interaction component such as a first interaction domain and the effector domain can be linked to a second interaction component such as a second interaction domain, such that the first and second interaction domain interact.

As used herein, the term “interaction domain” means a sequence motif in a first polypeptide (e.g. first dimer interaction domain), that is capable of interacting with a binding partner comprising a sequence motif in a second polypeptide (e.g. second dimer interaction domain) to operably link the first polypeptide and second polypeptide. In particular, the term is intended to encompass a first or second interaction dimer domain which together form a heterodimer pair that dimerizes for example under suitable inducing conditions. Other interaction domains are specifically contemplated and can be identified by the skilled person depending on the desired characteristics. Suitable inducible interaction domain pairs include, without limitation: FKBP/FRB (FK506 binding protein/FKBP rapamycin binding), which can be induced with e.g. rapamycin or AP21967; PYL/ABI which can be induced e.g. with abscisic acid; GID1/GAI, which can be induced with e.g. gibberellin or gibberellic acid; and pMag/nMag, which can be induced by e.g. blue light and/or temperature.

As used herein, “DNA targeting domain” refers to a polypeptide domain which binds DNA under DNA binding conditions, thereby targeting the polypeptide to said DNA. The DNA targeting domain can be any suitable DNA binding domain, for example an enzymatically inactive sequence-specific DNA targeting protein such as a CRISPR-Cas protein, (e.g. dCas9, dCas12, or other Cas-family proteins), a zinc-finger DNA binding domain, a transcriptional activator-like effector (TALE) DNA binding domain, bromodomains, chromodomains, Tudor domains, WD40 domains, PHD domains, PWWP domains, or other DNA-binding domains (DBDs) from eukaryotes or prokaryotes (e.g. Forkhead, basic helix-loop-helix, leucine zipper, homeodomain, nuclear hormone receptor, or a tet-repressor), or variants thereof. The DNA targeting domain may bind DNA in a sequence specific manner (e.g. Cas-family proteins, zinc-finger DNA binding domains, TALE DNA binding domains) or may bind to specific chromatin modifications (e.g. bromodomains (for acetylated histones) or chromodomains, Tudor domains, WD40 domains, PHD domains, PWWP domains etc. (for methylated histones). The DNA targeting domain may be a natural (e.g. non-engineered) DNA binding domain, such as for example a DNA binding domain found in a naturally occurring (e.g. endogenous) transcription factor, or the DNA targeting domain may be engineered for example to provide custom sequence specificity (e.g. sequence specificity that differs from the non-engineered DNA binding domain) or altered DNA binding affinity. Methods of engineering for example zinc finger DNA binding domains and TALE DNA binding domains to provide custom DNA binding specificity are known in the art, for example in Maeder et al. 2008 and Sanjana et al. 2012. Enzymatically active Cas9 can also be used when it would lead to repression, for example when the guide is a truncated guide (see for example [24]). The DNA targeting domain may have inherent target sequence specificity, for example in the case of zinc-finger DNA binding domains and TALE DNA binding domains, or target sequence specificity may be mediated by additional sequence-specific factors such as e.g. a guide RNA in the case of CRISPR-Cas proteins. Suitable DNA binding conditions depend on the DNA targeting domain and may include for example the presence of additional factors, such as for example tetracycline in the case in the case of tet-repressors, or a guide RNA in the case of Cas-family proteins.

The term “effector domain” as used herein refers to a polypeptide domain comprising at least one transactivation domain (TAD) described herein, for example the TADs listed in Tables 1-5 and functional variants thereof such as active fragments thereof. Optionally the effector domain may comprise two or more, for example two, three, four, or more transactivation domains described herein. In the activators described herein, the active fragment can be about 15 amino acids, about 20 amino acids, about 30 amino acids, about 40 amino acids, about 50 amino acids, about 60 amino acids, about 70 amino acids, or any number between 15 and 70 amino acids, or more than 70 amino acids. For example, for HSF1, the active fragment may comprise GFSVDTSALLDLFSP (SEQ ID NO: 104) which corresponds to amino acids 406 to 420 of HSF1. Accordingly, by way of example, the active fragment of HSF1 may comprise amino acids 401 to 427 of HSF1. Active fragments of other TADs can be identified by any suitable methods, for example using the methods described herein.

The heterologous transcriptional activator can be an effector N-terminal or a C terminal fusion, for example the order of the fusion can be effector domain—DNA targeting domain or DNA targeting domain—effector domain (see for example [25], [26], [27] and [28]). The effector domain can be fused to the DNA targeting domain by way of a linker. Similarly, two or more TADs may be fused together by way of one or more linkers. For example, glycine and glycine serine linkers can be used. Transcriptional activators described in the Examples used a variety of glycine serine linkers for example SGGSGGS (SEQ ID NO: 6), GGS, SGGS (SEQ ID NO: 7), and/or GSGSGS (SEQ ID NO: 8). Other linkers can also be used for example INSRSSGS (SEQ ID NO: 9).

The terms “CRISPR-Cas” or “Cas” as used herein refer to a CRISPR Clustered Regularly Interspaced Short Palindromic Repeats-CRISPR associated (CRISPR-Cas) protein that binds RNA and is targeted to a specific DNA sequence by the RNA to which it is bound. The CRISPR-Cas is a class II monomeric Cas protein for example a type II Cas such as Cas9. The Cas9 protein may be Cas9 from Streptococcus pyogenes, Francisella novicida, A. Naesulndii, Staphylococcus aureus or Neisseria meningitidis. Optionally the Cas9 is from S. pyogenes. The Cas protein can also be Cas12a (e.g. dCas12a) for example from Acidaminococcus sp., Lachnospiraceae bacterium, or Francisella tularensis (these have been shown to work as dCas variants), CasCD (Cas12j) and CasX (Cas12e) may also be used.

As used herein, the term “dCas9” refers to an enzymatically inactive (or dead) Cas9, which lacks DNA endonuclease activity but retains target DNA binding activity. For example, the dCAS9 comprises the sequence of CAS9 and D10A/H840A mutations in the RuvC1 and HNH nuclease domains. Optionally the dCas9 is a protein comprising an amino acid sequence with at least 80%, at least 90%, at least 95%, at least 99% or 100% sequence identity to a protein encoded by SEQ ID NO: 1 and comprising D10A/H840A mutations and retaining Cas9 target DNA binding activity (e.g. binding the gRNA and the target site). Similarly dCas12a refers to an enzymatically inactive Cas12a.

The terms “guide RNA,” “guide,” or “gRNA” as used herein refer to an RNA molecule that hybridizes with a specific DNA sequence and minimally comprises a spacer sequence. The guide RNA may further comprise a protein binding segment that binds a CRISPR-Cas protein. The portion of the guide RNA that hybridizes with a specific DNA sequence is referred to herein as the nucleic acid-targeting sequence, or spacer sequence. The protein binding segment of the guide may comprise for example a tracrRNA and/or a direct repeat. The term “guide” or “guide RNA” may refer to a spacer sequence alone, or an RNA molecule comprising a spacer sequence and a protein binding segment, according to the context. The guide RNA can be represented by the corresponding DNA sequence. The guide can be a truncated guide, for example comprising 15 or fewer nucleotides of complementarity to a target site as described in [24] when the enzyme is Cas9. For example, when Cas9 interacts with a truncated guide, Cas9's DNA binding capability remains intact while its nucleolytic activity is eliminated. Any length of guide that maintains Cas binding capability can be used.

The term “spacer” or “spacer sequence” as used herein refers to the portion of the guide that forms, or is capable of forming, an RNA-DNA duplex with the target sequence or a portion thereof. The spacer sequence may be complementary or correspond to a specific CRISPR target sequence. The nucleotide sequence of the spacer sequence may determine the CRISPR target sequence and may be designed or configured to target a desired CRISPR target site.

The term “tracrRNA” as used herein refers to a “trans-encoded crRNA” which may, for example, interact with a CRISPR-Cas protein such as Cas9 and may be connected to, or form part of, a guide RNA. The tracrRNA may be a tracrRNA from for example S. pyogenes. A tracrRNA may have for example the sequence of 5′-gtttcagagctatgctggaaacagcatagcaagttgaaataaggctagtccgttatcaacttgaaaaagtggcaccgagtcggtg c-3′ (SEQ ID NO: 2). Other tracrRNAs may also be used. Suitable tracrRNAs can be identified by a person skilled in the art, including for example 5′-GTTTTAGAGCTAGAAATAGCAAGTTAAAATAAGGCTAGTCCGTTATCAACTTGAAAAAGT GGCACCGAGTCGGTGC-3′ (SEQ ID NO: 3) or 5′-GTTTCAGAGCTACAGCAGAAATGCTGTAGCAAGTTGAAAT-3′ (SEQ ID NO: 4).

The terms “CRISPR target site” or “CRISPR-Cas target site” as used herein mean a nucleic acid to which an activated CRISPR-Cas protein (e.g. a CRISPR-Cas protein such as dCas9 bound to a guide RNA) will bind under suitable conditions. A CRISPR target site comprises a protospacer-adjacent motif (PAM) and a CRISPR target sequence (i.e. corresponding to the spacer sequence of the guide to which the activated CRISPR-Cas protein is bound). The sequence and relative position of the PAM with respect to the CRISPR target sequence will depend on the type of CRISPR-Cas protein. For example, the CRISPR target site of Cas9 or dCas9 may comprise, from 5′ to 3′, a 15 to 25, 16 to 24, 17 to 23, 18 to 22, or 19 to 21 nucleotide, optionally a 20 nucleotide target sequence followed by a 3 nucleotide PAM having the sequence NGG. Accordingly, a Cas9 target site may have the sequence 5′-N₁NGG-3′, where N₁ is 15 to 25, 16 to 24, 17 to 23, 18 to 22, or 19 to 21 nucleotides in length, optionally 20 nucleotides in length.

The CRISPR target site can be in any suitable genomic locus. For example, the CRISPR target site can be in a promoter, enhancer, 3′UTR, or other regulatory element, in a gene, optionally an intron or exon, in a locus corresponding to a non-coding RNA, or in an intergenic region. Optionally, the CRISPR target site is in a promoter or an enhancer.

Target DNA located in the nucleus of a cell requires a transcriptional activator that can enter the nucleus. Accordingly, the transcriptional activator may be nuclear-localized and/or may comprise for example one or more nuclear localization signals (NLS), optionally one or more SV40 NLSs. Optionally the transcriptional activator comprises two or more NLSs. Optionally the transcriptional activator may comprise one or more N-terminal NLSs, one or more C-terminal NLSs, one or more internal NLSs, or one or more N-terminal, one or more C-terminal NLSs, and/or one or more internal NLSs. Other configurations are specifically contemplated. In an embodiment, the NLS is an SV40 NLS having the sequence PKKKRKV (SEQ ID NO: 22). In an embodiment, the NLS further comprises an N- and/or C-terminal linker such as INSRSSGS (SEQ ID NO: 9), and optionally has the sequence INSRSSGSPKKKRKVGS (SEQ ID NO: 141).

The transcriptional activator can also be labelled with a tag. For example, suitable tags include but are not limited to Myc, FLAG, HA, V5, ALFA, T7, 6×His, VSV-G, S-tag, AviTag, StrepTag II, CBP, GFP, mCherry. The label can be fused at the N-terminus, the C-terminus or between two components of the heterologous transcriptional activator such as between the DNA targeting domain and the effector domain.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Similarly, it is specifically contemplated herein that the phrase “one or more” in reference to a group of elements includes at least one member of the stated group but not necessarily including one of each of the members of the stated group. For example, where an element comprises one or more of group members A, B, and/or C, the element may comprise A; B; C; A and B; A and C; B and C; or A, B, and C. Additional members not specifically listed in the group may also be present, for example with reference to the example above, the element may additionally comprise unlisted member D, and accordingly may comprise A and D; B and D; A, C and D; etc.

The term “about” as used herein means plus or minus 10%-15%, 5-10%, or optionally about 5% of the number to which reference is being made.

It should also be understood that, in certain methods described herein that include more than one step or act, the order of the steps or acts of the method is not necessarily limited to the order in which the steps or acts of the method are recited unless the context indicates otherwise. Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

Although any materials and methods similar or equivalent to those described herein can also be used in the practice or testing of the present disclosure, the following materials and methods are now described.

### II. Materials and Methods

Described herein is a collection of heterologous transcriptional activators, comprising one or more transactivation domains (TADs), and combinations thereof (“effector domains”) that can be operably linked to a DNA targeting domain to generate a heterologous transcriptional activator, and which can be used to activate gene expression of a desired gene, including an endogenous gene, for example for therapeutic purposes. In the heterologous transcriptional activators described herein, effector domains are operably linked to a DNA-targeting domain that can direct binding of the fusion construct to any locus in the genome. As demonstrated in the Examples, heterologous transcriptional activators comprising dCas9 or rTetR functionally associated with an effector domain comprising any one of the TADs listed in Table 1, active fragments thereof, for example those of SEQ ID NOs: 100-104, 107-115, 118-119, 156-160, 162, 164, and 166-185, and combinations of two or more of the TADs or active fragments thereof, for example as listed in Table 3, can be used to activate transcription of a target gene. The TAD or active fragment thereof may additionally comprise a linker and/or additional natural sequence e.g. 1, 2, 3, 4, 5, 6, 7 or more, for example up to 5, up to 10, up to 20, up to 30, or up to 40 (or any number in between), N- and/or C-terminal amino acids on the end of the TAD or active fragment. For example, the TAD labeled “ZXDC short” in Table 3 (SEQ ID NO: 107) comprises a 40 amino acid fragment found in both ZXDC-12 and 13 and comprises an additional 6 N-terminal amino acids of ZXDC-12 natural sequence and an additional 5 C-terminal amino acids of ZXDC-13 natural sequence. In an embodiment, the active fragment may be shorter than a fragment identified. For example, the active fragment may be 20 amino acids, or 25 amino acids of the 40 amino acid fragment found in for example both ZXDC-12 and ZXDC-13, optionally for example an internal portion of SEQ ID NO: 107. Shorter active fragments are exemplified for example by SEQ ID NO: 119 which comprises a 20 amino acid fragment found in both HSF1-20 and HSF1-21. Active fragments can be identified as described elsewhere herein. For example, the minimal fragment can be identified by comparing active fragments, for example for ATF6, the overlapping fragment shown in Table 5 is HRLDEDWDSALFAELGYFTDTDELQLEAANETYENNFDNL and for KLF7 the overlapping fragment is YFSALPSLEETWQQTCLELERYLQTEPRRISETFGEDLDC.

Accordingly, one aspect of the disclosure includes a heterologous transcriptional activator comprising a DNA targeting domain, and an effector domain comprising at least one TAD selected from the group consisting of any one of the TADs listed in any one of Tables 1-6, optionally Table 1 or optionally Table 2 or Table 6, active fragments thereof, and combinations thereof for example at least two TADs selected from the TADs listed in Table 1 or Table 3, or functional variants thereof, preferably at least one TAD selected from the TADs listed in Table 4, or Table 5 or Table 6, and/or functional variants thereof, wherein the DNA targeting domain and effector domain are operably linked.

In an embodiment, the at least one TAD is selected from Table 1.

In an embodiment, the at least one TAD is selected from Table 2.

In an embodiment, the at least one TAD is selected from Table 3.

In an embodiment, the at least one TAD is selected from Table 4.

In an embodiment, the at least one TAD is selected from Table 5.

In an embodiment, the at least one TAD is selected from Table 6.

It is understood that the at least two TADs or functional variants thereof can be selected from any Table, for example one from each of Table 3 and Table 4, two or more, for example 3 or 4, from Table 3 etc. It is also contemplated that the grouping can include any sub-combination of the TADS described in any of Tables 1-6, for example one or more TADs may be excluded.

The DNA targeting domain and the effector domain may be operably linked by covalent linkage, for example as domains of a single polypeptide, and/or may be operably linked via one or more interaction components such as interaction domains and/or interact under certain conditions. Accordingly, in one embodiment, the heterologous transcriptional activator is a single polypeptide. In another embodiment, the heterologous transcriptional activator further comprises a pair of (i.e. a first and a second) interaction domains, optionally dimer interaction domains, optionally a pair of inducible dimer interaction domains that dimerize under suitable conditions. For example, the heterologous transcriptional activator may comprise a first polypeptide comprising a DNA targeting domain and a first dimer interaction domain, optionally an inducible dimerization domain, and a second polypeptide comprising an effector domain and a second dimer interaction domain, optionally an inducible dimerization domain, wherein the first dimer interaction domain and second dimer interaction domain interact, optionally the first inducible dimerization domain and second inducible dimerization domain interact in the presence of one or more inducing agents.

As shown in the Examples, the dimerization of a heterologous transcriptional activator comprising ABI1 and PYL1 may be induced with the addition of abscisic acid. Accordingly, in an embodiment, the transcriptional activator comprises a first and second inducible dimerization domain that provide for inducible transcriptional activation in the presence of an inducing agent. The skilled person can readily identify and select suitable inducible dimerization domains that may be used together. Any suitable inducible dimerization domains may be used, for example the dimerization of ABI1 and PYL1 may be induced with the addition of abscisic acid. Other inducible systems include those based on induction with rapamycin, gibberellic acid/gibberellin, and split dCas9-based systems. For example, dimerization of GID1 and GAI can be induced by gibberellin, and dimerization of FKBP and FRB can be induced with rapamycin or its analogs, e.g., rapalogs. Higher-order multimerization systems, such as the SunTag system (Tenenbaum et al., 2014) are also contemplated herein.

Interaction between the DNA targeting domain and effector domain can also be controlled using other inducible systems. Other systems (that are not dependent on dimerization) include grazoprevir-induced stabilization (Tague et al. 2018) or tamoxifen-regulated nuclear localization using estrogen receptor ligand binding domain variants. In the case of grazoprevir-induced stabilization, the DNA targeting domain and effector domain would be linked by a self-cleaving NS3 protease domain. Only in the presence of grazoprevir (which inhibits NS3 activity), DNA targeting domain and effector domain would stay together and regulate gene expression.

The DNA targeting domain can be selected from a variety of DNA binding domains, for example a zinc finger DNA binding domain, transcriptional activator-like effector (TALE) DNA binding domain, dCas9, dCas12 or other Cas-family proteins, or other DNA-binding domains (DBDs) from eukaryotes or prokaryotes (e.g. Forkhead, basic helix-loop-helix, leucine zipper, homeodomain, nuclear hormone receptor, or a tet-repressor), or variants thereof. The DNA targeting domain may be a natural (e.g. non-engineered) DNA binding domain, such as for example a DNA binding domain found in a naturally occurring (e.g. endogenous) transcription factor, or the DNA targeting domain may be engineered for example to provide custom sequence specificity (e.g. sequence specificity that differs from the non-engineered DNA binding domain) or altered DNA binding affinity. Methods of engineering for example zinc finger DNA binding domains and TALE DNA binding domains to provide custom DNA binding specificity are known in the art, for example in Maeder et al. 2008 and Sanjana et al. 2012. In the case where a heterologous transcriptional activator described herein comprises a DNA targeting domain comprising a natural DNA-binding domain, the effector domain would be targeted to all loci that the transcription factor endogenously binds to, thereby augmenting/replacing the function of the endogenous transcription factor. For example, it is known that replacing Oct4 transactivation domain with VP16 increases the efficiency of reprogramming fibroblasts to iPS cells. Similarly, a heterologous transcriptional activator comprising a natural DNA binding domain operably linked to an effector domain could promote e.g., wound healing, transdifferentiation, or tissue regeneration by activating transcription of target genes that are regulated by the endogenous transcription factor. In the case of engineered (e.g. custom sequence specificity) zinc finger DNA binding domains, TALE DNA binding domains or Cas family proteins, an effector domain could be brought to one or more specific loci, or optionally a single locus in the genome in a controlled manner.

In an embodiment, the DNA targeting domain comprises a CRISPR-Cas protein such as dCas9. Enzymatically inactive CRISPR-Cas proteins which retain gRNA and target DNA binding activity can be used. For example, mutation of D10A/H840A in Cas9 introduces mutations in the RuvC1 and HNH nuclease domains and results in inactivation. In an embodiment, the CRISPR-Cas protein is dCas9 having an amino acid sequence of SEQ ID NO: 1 or an amino acid sequence with at least 80%, at least 90%, at least 95%, or at least 99% sequence identity to SEQ ID NO: 1 and comprises D10A/H840A and which retains gRNA and target DNA binding activity. Other enzymatically inactive CRISPR-Cas proteins are also contemplated can be identified by the skilled person.

In an embodiment, the DNA targeting domain comprises a zinc finger DNA binding domain. In an embodiment, the zinc finger DNA binding domain is an engineered zinc finger DNA binding domain which has been engineered to bind a specific DNA sequence.

The effector domain comprises at least one transactivation domain (TAD) described herein, or an active fragment thereof. As shown in the Examples, various full-length ORFs and TADs identified herein can be used, alone or in combination, to activate transcription of a GFP reporter construct or an endogenous gene such as CD133. Also shown in the Examples, the effector domain can comprise at least one TAD domain shown in Table 1, and/or an active fragment thereof, such as for example as shown in Table 3. Accordingly, in an embodiment, the effector domain comprises at least one TAD shown in Table 2, Table 4, and/or Table 5 and/or Table 6 and/or a functional variant of any one thereof, or two or more TADs shown in Table 1, Table 2, Table 3, Table 4, Table 5, and/or Table 6 and functional variants of any one thereof. In an embodiment, the TAD comprises a polypeptide having a sequence with at least 80%, at least 90%, at least 95%, or at least 99% sequence identity to any one of the TAD domains in Table 1, Table 2, Table 3, Table 4, Table 5, and/or Table 6 and functional variants of any one thereof, and which retains (e.g. is as at least 80% as effective at) transcriptional activation activity and/or interaction with specific transcriptional co-activators such as for example CBP/p300, NuA4, and/or BRD4.

Variants and combinations thereof may also be used. In an embodiment, the effector domain comprises two or more tandem TADs, optionally two TADs, three TADs, four TADs, or more than four TADs, for example 5 TADs, 10 TADs, 15 TADs, 20 TADs, 25 TADs, 30 TADs, or any number of TADs between 5 TADs and 30 TADs, or more than 30 TADs. In an embodiment, the effector domain comprises two or more TADs or functional variants thereof selected from those listed in Table 1, Table 2, Table 3, Table 4, Table 5, and/or Table 6 and functional variants of any one thereof. In an embodiment, the effector domain comprises three or four TADs selected from those listed in Table 3. In an embodiment, the effector domain comprises one or more of SEQ ID NO: 185, SEQ ID NO: 103, SEQ ID NO: 167, SEQ ID NO: 105, SEQ ID NO: 106, and/or SEQ ID NO: 104. In an embodiment, the effector domain comprises SEQ ID NO: 185, optionally SEQ ID NO: 90, 91, 102, or 157. In an embodiment, the effector domain comprises SEQ ID NO: 103, optionally SEQ ID NO: 46, 47, or 162. In an embodiment, the effector domain comprises SEQ ID NO: 167, optionally SEQ ID NO: 101, 110, 166, or 172. In an embodiment, the effector domain comprises SEQ ID NO: 105, optionally SEQ ID NO: 116, 117, or 165. In an embodiment, the effector domain comprises SEQ ID NO: 106, optionally SEQ ID NO: 116 or 117. In an embodiment, the effector domain comprises SEQ ID NO: 104, optionally SEQ ID NO: 118, 119, or 159. In an embodiment, the effector domain comprises SPDYE4-CITED1-RELA-HSF1 (SEQ ID NO: 121); SPDYE4-CITED1-RELA (SEQ ID NO: 123); HSF1-RELA-SPDYE4-CITED1 (SEQ ID NO: 125); SPDYE4-CITED1-p65-miniHSF1 (SEQ ID NO: 127); miniSPDYE4-CITED1-p65-HSF1 (SEQ ID NO: 129); SPDYE4-miniCITED1-p65-HSF1 (SEQ ID NO: 131); SPDYE4-CITED1-minip65(C)-HSF1 (SEQ ID NO: 133); or SPDYE4-CITED1-minip65(N)-HSF1 (SEQ ID NO: 135). In an embodiment, the effector domain comprises SPDYE4-CITED1-SERTAD2-HSF1; SPDYE4-CITED1-KLF6-HSF1; SPDYE4-CITED1-ZXDC-HSF1; SPDYE4-CITED1-ATF6-HSF1; SPDYE4-CITED1-FOXO1-HSF1; SPDYE4-CITED1-ATMIN-HSF1; SPDYE4-CITED1-p65-SERTAD2; SPDYE4-CITED1-p65-KLF6; SPDYE4-CITED1-p65-ZXDC; SPDYE4-CITED1-p65-ATF6; SPDYE4-CITED1-p65-FOXM1; SPDYE4-CITED1-p65-ATMIN; SPDYE4-C3orf62-p65-HSF1; SPDYE4-DDIT3-p65-HSF1; SPDYE4-FOXO1-p65-HSF1; SPDYE4-ATMIN-p65-HSF1; SPDYE4-ZXDC-p65-HSF1; C3orf62-CITED1-p65-HSF1; C11orf74-CITED1-p65-HSF1; KLF6-CITED1-p65-HSF1; ZXDC-CITED1-p65-HSF1; or SOX7-CITED1-p65-HSF1. In an embodiment, the effector domain comprises SPDYE4-C3orf62.2-P_AD-HSF1 (SEQ ID NO: 174); SPDYE4-C3orf62.3-P_AD-HSF1 (SEQ ID NO: 176); SPDYE4-C3orf62_MT-P_AD-HSF1 (SEQ ID NO: 178); SPDYE4-DDIT3_MT-P_AD-HSF1 (SEQ ID NO: 180); SPDYE4-CITED1-P_AD-HSF1_MT (SEQ ID NO: 182); or 3×ZNF473_KRAB (SEQ ID NO: 184). Other combinations are specifically contemplated herein.

The effector domain may comprise two or more TADs with different transcriptional co-activator preferences. For example, the effector domain may comprise a TAD which interacts with CBP/p300 components for example a FOXO TAD, and a TAD which interacts with BET components for example a SPDYE4 TAD. The effector domain may comprise two or more TADs with similar transcriptional co-activator preferences. For example, the effector domain may comprise two TADs which interact with CBP/p300 components, for example the effector domain may comprise a FOXO1 TAD and a CITED1 TAD. Other combinations are specifically contemplated herein.

With respect to functional variants, “as effective” as used herein means the functional variant retains at least 80%, at least 85%, at least 90%, at least 95%, at least 96%, at least 97%, at least 98%, at least 99%, 100%, or more than 100% transcriptional activation activity and/or co-activator interaction compared to the unmodified or non-variant TAD (e.g. wild-type or full-length TAD). Transcriptional activation activity and/or co-activator interaction of variants such as truncations can be determined for example using the methods described herein. For example transcriptional activation activity can be determined using the GFP reporter system described in the Examples. Variants can be tethered to the same reporter or endogenous context while controlling for expression levels of each DNA targeting domain (e.g. dCas9). Any differences detected in induced expression of the reporter or target genes when compared to the parental TAD can be contributed to the effect of the variant. Co-activator interaction can be determined for example by AP-MS and/or BiolD e.g. as shown in the Examples.

Exemplary TAD and effector domain nucleic acids and polypeptides are provided in Tables 1-6 and SEQ ID NOs: 120-135 and 173-184. In an embodiment, the effector domain may comprise an amino acid sequence encoded by said nucleic acids, or an amino acid sequence with at least 80%, at least 90%, at least 95%, at least 96%, at least 97%, at least 98%, or at least 99% sequence identity to an amino acid sequence encoded by the TAD of SEQ ID NOs: 120-135 and 173-184. The activity of the encoded polypeptides (fusion or when expressed and activated) of such polypeptides is as effective (e.g. provides at least 80% as effective transcriptional activation) as for example SEQ ID NOs: 120-135 and 173-184.

In an embodiment, the effector domain is fused to the DNA targeting domain by way of a linker. In an embodiment, two or more TADs are fused together by way of one or more linkers. For example, glycine and glycine serine linkers can be used. Transcriptional activators described in the Examples used a variety of glycine serine linkers for example SGGSGGS (SEQ ID NO: 6), GGS, SGGS (SEQ ID NO: 7), and/or GSGSGS (SEQ ID NO: 8). Other linkers can also be used for example INSRSSGS (SEQ ID NO: 9).

In an embodiment, the transcriptional activator comprises one or more nuclear localization signals (NLS). Any suitable NLS can be used. Optionally the NLS is an SV40 NLS. The one or more NLS can be one or more N-terminal NLS, one or more C-terminal NLS, one or more internal NLS, and/or combinations thereof. Optionally, the NLS may comprise an NLS of SEQ ID NO: 22. In an embodiment, the NLS further comprises an N- and/or C-terminal linker such as INSRSSGS (SEQ ID NO: 9), and optionally has the sequence INSRSSGSPKKKRKVGS (SEQ ID NO: 141).

As described herein, the transcriptional activator or effector domain may be encoded by a nucleic acid and/or expressed from an expression construct. Accordingly, one aspect of the disclosure is a nucleic acid encoding a transcriptional activator described herein. Another aspect of the disclosure is a nucleic acid encoding an effector domain of a transcriptional activator described herein. For example, the nucleic acid may encode a TAD as provided in any one of Tables 1 to 6, optionally Tables 2, 4, 5 and/or 6, optionally Table 2 or Table 4 or Table 6, or two or more TADs as provided in Tables 1-6. In an embodiment, the nucleic acid may comprise a nucleic acid of any one of SEQ ID NOs: 120, 122, 124, 126, 128, 130, 132, 134, 173, 175, 177, 179, and 180, or a sequence with at least 80%, at least 90%, at least 95%, at least 96%, at least 97%, at least 98%, at least 99% or 100% sequence identity to SEQ ID NOs: 120, 122, 124, 126, 128, 130, 132, 134, 173, 175, 177, 179, and 180, wherein the heterologous transcriptional activator, for example activates transcription about as effectively as the effector domains encoded by SEQ ID NO: 120, 122, 124, 126, 128, 130, 132, 134, 173, 175, 177, 179, and 180, for example at least 80% as effectively, at least 85% as effectively, or at least 90% as effectively, at least 95% as effectively, at least 96% as effectively, at least 97% as effectively, at least 98% as effectively, at least 99% as effectively, at least 100% as effectively, or more than 100% as effectively for example as assessed in an assay as described herein. The sequence identity is for example relative to the full effector domain sequence or relative to one or more TADs or TAD fragments encoded therein. Other portions, linkers, NLS etc. can be completely different. The nucleic acid encoding the effector domain may be suitable for generating a nucleic acid encoding a transcriptional activator described herein. For example, the nucleic acid encoding the effector domain may be flanked by suitable cloning sites, or an expression construct or vector comprising said nucleic acid may comprise a cloning site to facilitate insertion of a DNA targeting domain to operably link the effector domain and DNA targeting domain.

The term “cloning site” as used herein refers to a portion of a nucleic acid molecule into which a nucleic acid molecule of interest may be inserted, or to which a nucleic acid molecule of interest may be joined, using recombinant DNA technology (cloning). In the context of an expression cassette, the cloning site may be located between the promoter and the polyadenylation signal, such that a nucleic acid molecule of interest may be cloned into the expression cassette in operable linkage with the promoter and the polyadenylation site. Several cloning techniques are known to the skilled person and the cloning site will include the necessary characteristics (such as restriction endonuclease site(s), recombinase recognition site(s), or blunt or overhanging end(s)) to allow insertion of the nucleic acid molecule of interest at the cloning site. The cloning site may, for example, be a multiple cloning site (MCS) or polylinker region comprising a plurality of unique restriction enzyme recognition sites to allow a nucleic acid molecule of interest to be inserted. Alternately, or in addition, the cloning site may include one or more recombinase recognition sites to allow DNA insertion by recombinational cloning; employing site-specific recombinase(s), such as Integrase or Cre Recombinase, to catalyze DNA insertion. Examples of recombinational cloning systems include Gateway® (Integrase), Creator™ (Cre Recombinase), and Echo Cloning™ (Ore Recombinase). For some cloning strategies, an expression cassette or vector may be provided as a linear molecule, allowing blunt or overhanging ends of a nucleic acid molecule of interest to be joined to blunt or overhanging ends of the expression cassette or vector, for example by ligation or polymerase activity, thus forming a circular molecule. In this case, the blunt or overhanging ends of the expression cassette or vector may together be viewed as the cloning site. Such an approach is commonly used to clone PCR products.

A related aspect is an expression construct comprising the nucleic acid encoding the transcriptional activator operably linked to a promoter and a transcription termination site. Any suitable promoter may be used. Suitable promoters can be identified by a person skilled in the art, and may include for example CMV, EF1A, or PGK. For example, the promoter and/or enhancer sequences of e.g. SEQ ID NOs: 25, 26, 27, and/or 28 can be used in an expression construct. Inducible promoters may also be used.

In one embodiment, the construct is a vector. Any suitable vector may be used. Suitable vectors can be identified by a person skilled in the art, and may include a viral vector, optionally a lentiviral vector or an adenoviral vector. Suitable vectors may comprise for example a promoter for expressing effector construct, polyA tail, 3′UTR elements like WPRE to increase stability of expression, insulator sequences, lentiviral packaging signals, a fluorescent protein, and/or an antibiotic resistance marker. Additional suitable components can be identified by a person skilled in the art.

In another embodiment, the transcriptional activator, nucleic acid, construct, or vector is in a cell. Any suitable cell may be used and can be determined by the skilled person on the basis of the desired application. The cell may be from any organism. Optionally the cell is a mammalian cell such as a human cell or a mouse cell. Optionally the cell is a cell line. The cell line may be any suitable cell line.

The transcriptional activator, nucleic acid, construct, or vector may be introduced into the cell in any suitable manner, for example by transfection. Suitable transfection reagents and methods are routinely practiced in the art and can be identified by the skilled person. Optionally, the construct is a viral vector, optionally a lentiviral vector, and is introduced into the cell by transduction. Suitable transduction methods are routinely practiced in the art and can be identified by the skilled person.

In some embodiments the cell is stably expressing the heterologous transcriptional activator, optionally the cell is stably transduced, for example prepared using a virus comprising a nucleic acid encoding the heterologous transcriptional activator.

Another aspect is a transcriptional activation system comprising the transcriptional activator described herein, a nucleic acid encoding the transcriptional activator, or construct or vector comprising said nucleic acid or a cell expressing the transcriptional activator. In the case of a system based on CRISPR-Cas, the system comprises at least one gRNA. In the case of a system based on inducible dimerization domains, the system optionally comprises at least one inducing agent.

Also provided is a composition comprising a heterologous transcriptional activator described herein, a nucleic acid described herein, a construct described herein, a vector described herein, a cell described herein and/or a transcriptional activation system described herein. The composition can comprise a carrier, such as BSA, or a diluent suitable according to the composition components, optionally water or buffered saline. The composition can comprise multiple components such as transcriptional activators, nucleic acids, constructs, vectors or cells comprising the same or different elements.

Also provided herein is a kit for example for activating transcription of a target gene or performing a method described herein, the kit comprising a transcriptional activator described herein, a nucleic acid, expression construct, or vector encoding a transcriptional activator described herein, or a cell expressing the transcriptional activator described herein, and optionally a vial housing the transcriptional activator, nucleic acid, expression construct, vector, cell or composition. The kit can comprise multiple of one or more of the aforementioned components. Optionally the kit comprises a gRNA expression construct, an inducing agent, and/or instructions for carrying out the methods described herein.

Also described herein are methods of activating transcription of a target gene in a cell. As demonstrated in the Examples, a transcriptional activator of the disclosure can be targeted to a genomic locus such as a promoter to activate transcription of a target gene in a cell.

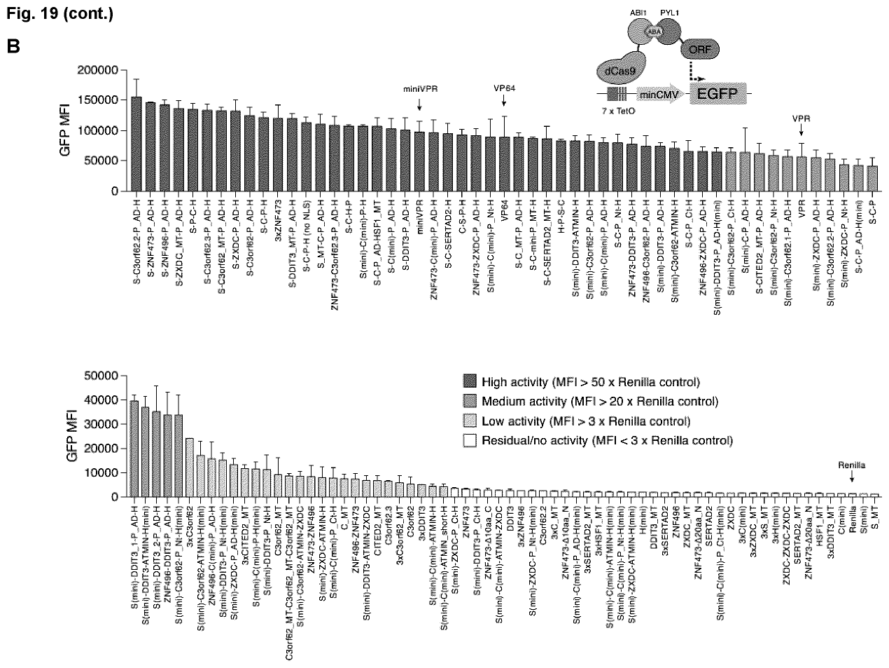

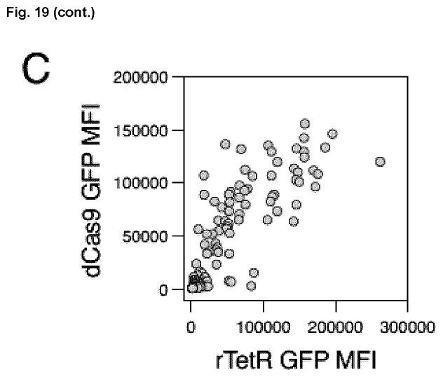

The transcriptional effectors identified herein may be full-length proteins, fragments thereof (transactivation domains), functional variants thereof or combinations of transactivation domains or functional variants thereof. They cover multiple different transcriptional activation strengths from very powerful to moderate to weak. This can be used to achieve a desired expression level of endogenous genes, particularly in cases where too high expression can cause deleterious phenotypes. Activation strength can be tuned by selecting different TADs or functional variants (e.g. active fragments) for inclusion in the effector domain. The activation strength of an effector domain can be determined by the skilled person for example using the MFI or percent GFP positive cells in the recruitment assays shown in the Examples described herein. For example, the relative strength of an effector domain can be determined by comparing the MFI or percent GFP positive cells of the specific effector domain in combination with a specific DNA targeting domain and specific DNA target relative to a control such as Renilla in combination with the same DNA targeting domain and specific DNA target. High activation can be considered to be for example at least or above 50× control, at least or above 75× control, at least or above 100× control, or at least or above 150× control. Medium activation can be considered to be for example at least or above 10×, at least or above 20×, at least or above 30×, or at least or above 40× control, and up to 50×, up to 75×, up to 100×, or up to 150× control. Low activation can be considered to be for example at least 2×, at least 2.5×, at least 3×, at least 4×, or at least 5× control and up to 10×, up to 20×, up to 30×, or up to 40× control. For example, as shown in FIGS. 19A and 19B, high activation may be considered to be >50-fold, medium may be 20× to 50×, and low may be at least 3× up to 20× relative to the Renilla control. Suitable activation levels may be selected depending on the desired application.

As described herein another level of control for transcriptional regulation can be added with chemically induced dimerization with e.g. rapalogs or abscisic acid. In this case, one half (e.g. a DNA targeting domain) would be fused to FKBP or PYL1, and the other half (e.g. the effector domain) fused to FRB or ABI1. Treatment with rapalog or abscisic acid would induce the interaction between FKBP and FRB or PYL1 and ABI1, respectively, leading to temporally regulated gene expression. As shown in the Examples, the dimerization of a heterologous transcriptional activator comprising ABI1 and PYL1 may be induced with the addition of abscisic acid. The skilled person can readily identify and select suitable inducible dimerization domains and inducing agents that may be used together. Any suitable inducible combination of protein dimerization domains and inducing agents may be used, for example the dimerization of ABI1 and PYL1 may be induced with the addition of abscisic acid. Other inducible systems include those based on induction with rapamycin, gibberellic acid/gibberellin, and split dCas9-based systems. For example dimerization of GID1 and GAI can be induced by gibberellin, and dimerization of FKBP and FRB can be induced with rapamycin or its analogs, e.g. rapalogs. Higher-order multimerization systems, such as the SunTag system (Tenenbaum et al., 2014) are also contemplated herein.

Interaction between the DNA targeting domain and effector domain can also be controlled using other inducible systems. Other systems (that are not dependent on dimerization) include grazoprevir-induced stabilization (Tague et al. 2018) or tamoxifen-regulated nuclear localization using estrogen receptor ligand binding domain variants. In the case of grazoprevir-induced stabilization, the DNA targeting domain and effector domain would be linked by a self-cleaving NS3 protease domain. Only in the presence of grazoprevir (which inhibits NS3 activity), DNA binding domain and effector domain would stay together and regulate gene expression.

Accordingly, one aspect of the disclosure is a method of activating expression of a target gene in a cell, the method comprising introducing into the cell a transcriptional activator described herein, and culturing the cell under suitable conditions such that the DNA targeting domain guides the transcriptional activator to the target site and the effector domain activates transcription of the target gene. In an embodiment, the target gene is an endogenous gene. In an embodiment, where the transcriptional activator comprises CRISPR-Cas, the method further comprises introducing into the cell at least one gRNA that targets a desired genomic locus in the cell, and culturing the cell under suitable conditions such that the at least one gRNA associates with the CRISPR-Cas protein and guides the CRISPR-Cas protein to guide the transcriptional activator to a CRISPR target site such that the effector domain activates transcription of the target gene. In an embodiment where the transcriptional activator comprises an inducible dimerization domain in each of the DNA targeting domain and in the effector domain, the method further comprises introducing into the cell at least one inducing agent and culturing the cell under suitable conditions that the first and second inducible dimerization domains associate such that the at least one effector domain activates transcription of the target gene.

The methods described herein can be used to modulate gene expression of a target gene for example to induce expression of an endogenous gene or modulate chromatin opening in defined regions of the genome. By way of example, some TADs could promote chromatin opening in intergenic regions (i.e. not promoters or enhancers), which could lead to chromatin opening and rearrangement of chromosome folding.

The methods described herein can be used to identify or screen for one or more genomic loci that are important for cell viability or a phenotype of interest. By way of example, the methods described herein can be used to screen for genes or regulatory elements thereof that are important for resistance or sensitivity to a toxin of interest such as diphtheria toxin. In another example, the methods described herein can be used to identify regulatory elements that are important for expression of a protein of interest such as CD81. In a further example, the methods described herein can be used in high-throughput screening methods to identify essential or non-essential genes in a cell type by screening for gRNAs that are over- or under-represented in a cell population under certain conditions e.g. drug treatment over time. Other applications can be determined by a person skilled in the art.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### III. Examples

### Example 1. Platform for Identifying Transcriptional Activators in Human Cells

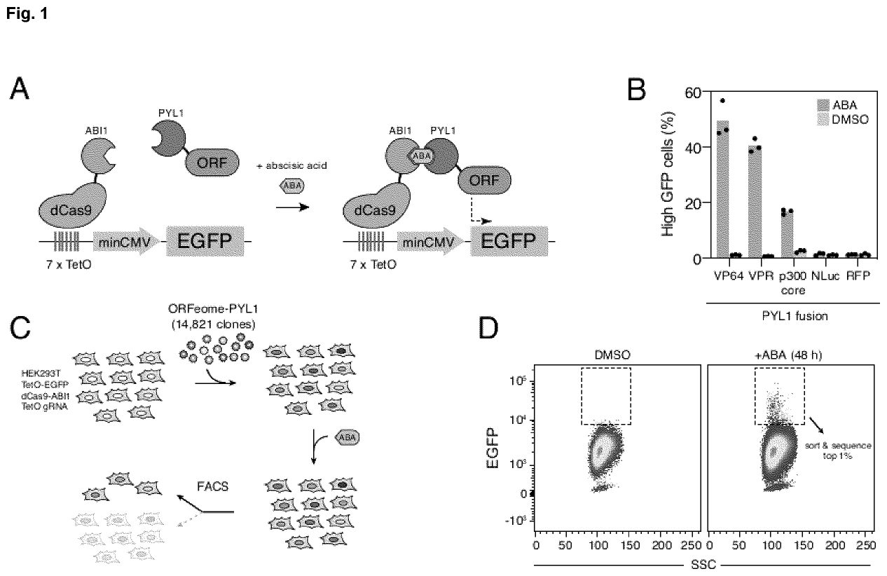

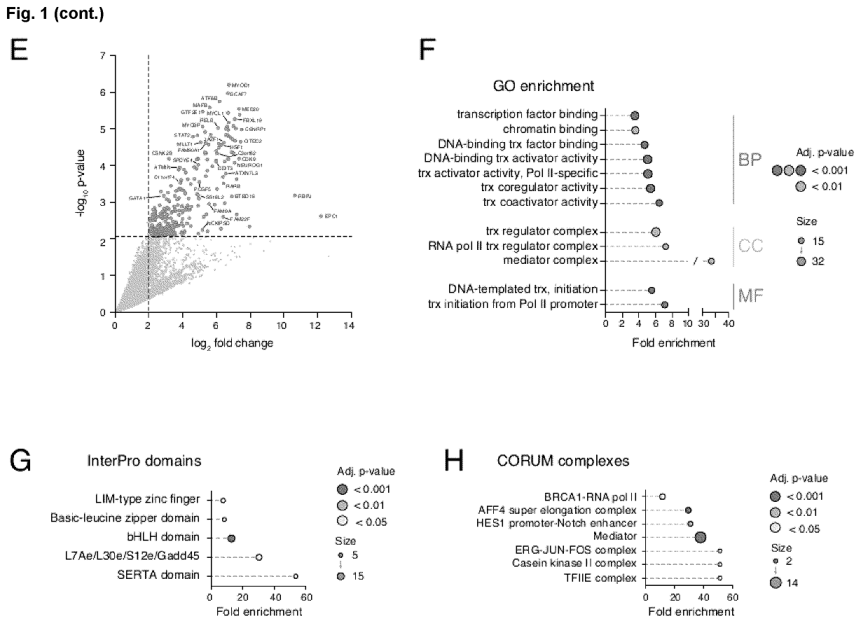

To identify transcriptional regulators in a systematic manner, a chemically-induced dimerization (CID) system was used, where catalytically inactive Cas9 (dCas9) is tagged in its N terminus with the protein phosphatase ABI1 and the potential transcriptional activator is fused to the abscisic acid receptor PYL1 (FIG. 1A)(Gao et al., 2016; Liang et al., 2011). Treatment of cells with abscisic acid (ABA) induces an interaction between ABI1 and PYL1, thereby recruiting the potential transcriptional activator to a specific genomic locus defined by the guide RNA (gRNA). As a reporter, a HEK293T cell line that contains a stably integrated construct with a 7×TetO array and a basal CMV promoter driving the expression of EGFP was used (Gao et al., 2016). A monoclonal cell line that expresses ABI1-dCas9 and a single gRNA targeting TetO was generated by lentiviral infection. Transfection of this cell line with known transcriptional activators VPR, VP64, and p300 fused to PYL1 led to robust induction of GFP upon ABA treatment (FIG. 1B), consistent with a previous report (Gao et al., 2016). Recruitment of luciferase or RFP to the promoter did not induce GFP expression, demonstrating that ABA alone does not activate the reporter (FIG. 1B).

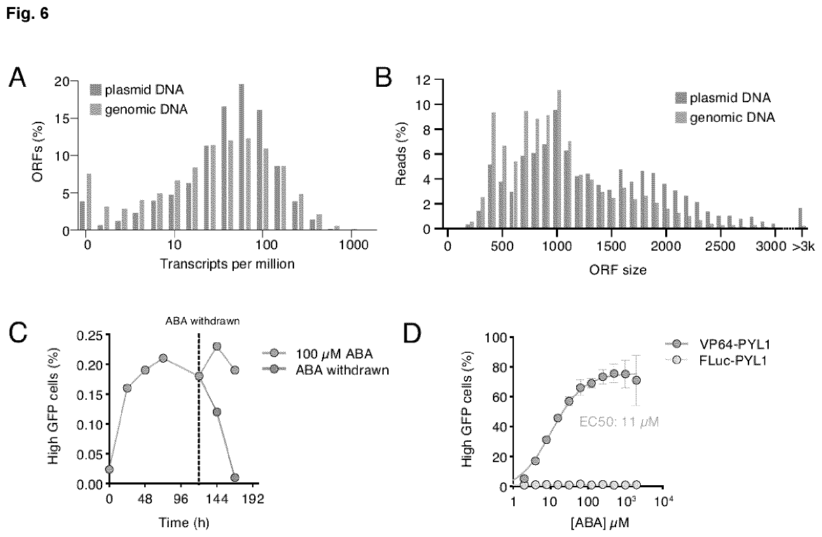

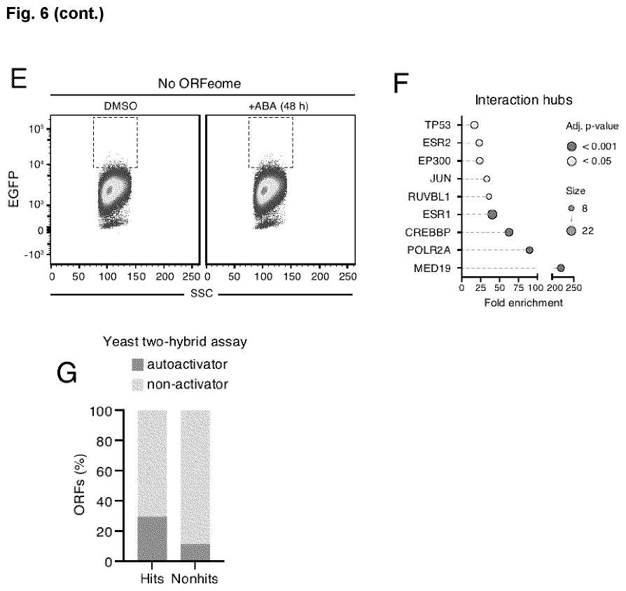

To scale up from individual clones, a pooled library of human ORFeome 8.1 and ORFeome collaboration clones (ORFeome Collaboration, 2016; Yang et al., 2011) in a lentiviral vector containing a C-terminal PYL1 was generated. Together, these open reading frame (ORF) libraries contain 14,821 clones corresponding to 13,571 unique genes. The reporter cell line was infected with the pooled library at low multiplicity of infection, thus ensuring that most cells were infected with only one lentivirus (FIG. 1C). The pooled library covered 96% and 93% of the ORFeome before and after infection, respectively, demonstrating wide coverage of the library despite significant diversity in insert size (FIGS. 6A and B). After treating the infected cells with ABA, a clear induction of GFP in a subset of cells was observed (FIG. 1D). Reporter induction was dependent on treatment time, ABA concentration, and presence of the ORFeome (FIGS. 6C-6E). Furthermore, withdrawal of ABA led to rapid loss of high GFP population (FIG. 6C), demonstrating that continuous promoter occupation is required for activation. To identify transcriptional activators, the top 1% of GFP positive cells of ABA treated cells was sorted in duplicate and the ORFs in the high GFP population were identified by sequencing (FIG. 1D).

Methods are as described in Example 7.

### Example 2. ORFeome-Wide Screen Identifies Known and Novel Transcriptional Activators

248 putative transcriptional activators were identified, using a cut-off of 5% false discovery rate and at least 4-fold change in read counts between top 1% GFP positive cells and unsorted cells (FIG. 1E). Gene ontology (GO) analysis revealed significant enrichment for multiple functional categories related to transcriptional activation (FIG. 1F). The hits were also highly enriched in protein domains found in many transcriptional regulators (FIG. 1G), subunits of chromatin-associated protein complexes (FIG. 1H), and in interactors of central hubs of transcription, such as RNA polymerase II and histone acetyltransferases CBP and p300 (FIG. 6F). Moreover, the hits were significantly overlapping with human proteins that function in yeast two-hybrid assays as autoactivators (11% in the proteome vs. 29% among the hits; p<0.0001, Fisher's exact test; FIG. 6G), which are proteins that activate reporter gene expression in yeast when ectopically recruited to the promoter (Luck et al., 2020). The screen results were also validated by assaying a collection of 90 hits and non-hits by individually transfecting them into the same reporter cell line. The results were highly consistent with the screen: almost all hits reproduced when tested individually, and conversely, most non-hits did not activate the reporter (FIG. 6H), suggesting low false positive and false negative rates in this setting. Together, these results indicate that the screen identified functionally relevant transcriptional activators in a reproducible manner.

Individual screen hits included well-characterized factors regulating distinct stages of transcription (FIG. 1E). For example, among sequence-specific transcription factor hits were known activators RELB and MYCL (Barrett et al., 1992; Ryseck et al., 1992) in addition to several master TFs regulating stress response, such as HSF1, ATF6, and DDIT3/CHOP (Vihervaara et al., 2018). Co-activators that do not bind DNA themselves but associate with TFs included STAT2, CITED1, and SERTAD1 (Bousoik and Montazeri Aliabadi, 2018; Hsu et al., 2001; Yahata et al., 2001). Both subunits of TFIIE (GTF2E1 and GTF2E2), a general transcription factor regulating the assembly of the pre-initiation complex (Cramer, 2019), were also prominent hits, as were proteins promoting RNA polymerase II release (the P-TEFb complex subunit CDK9) and transcriptional elongation (e.g., ELL3 and MLLT1)(Chen et al., 2018; Cramer, 2019). Other prominent activators included subunits of chromatin-modifying complexes such as SAGA (ATXN7L3, TADA3, SGF29) and NuA4 (EPC1, MGRBP), and GADD45 family proteins that mediate DNA demethylation (GADD45A, GADD45B, GADD45G)(Barreto et al., 2007). Finally, 13 of the 15 Mediator subunits that were present in the library were identified. These highlighted examples illustrate that the screen uncovered factors promoting transcription by multiple different mechanisms and at distinct stages of the transcription cycle.

In addition to known transcriptional regulators, a large collection of proteins were identified in the screen that have not been previously linked to transcription (e.g. C11orf74/IFTAP, NCKIPSD, DCAF7, HFM1) or are completely uncharacterized (e.g. C3orf62, FAM90A1, SPDYE4, SS18L2, FAM9A, C21orf58)(FIG. 1H). These results suggest that the human genome encodes many previously unknown transcriptional regulators. Detailed characterization of several of these factors is described below.

Methods are as described in Example 7.

### Example 3. Distinct Transcriptional Activities within TF Families

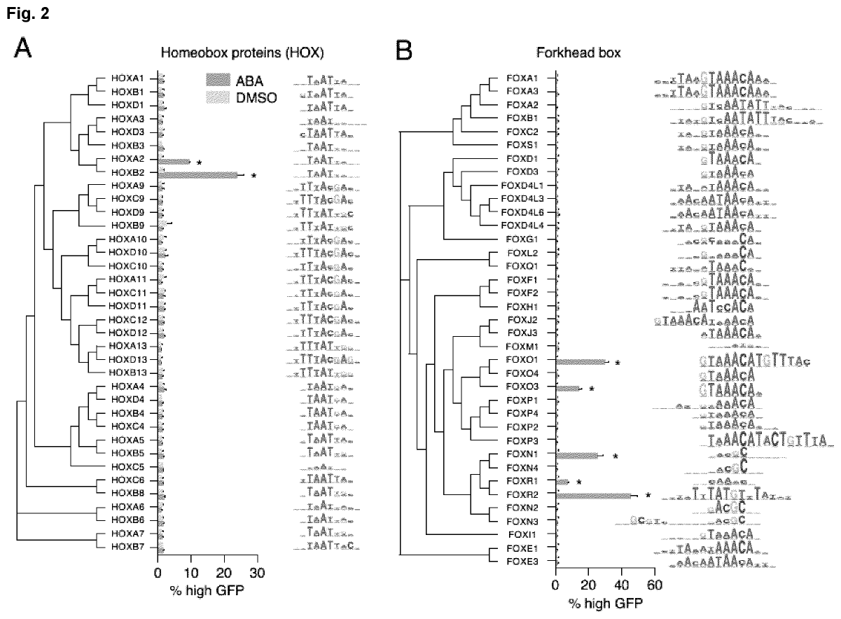

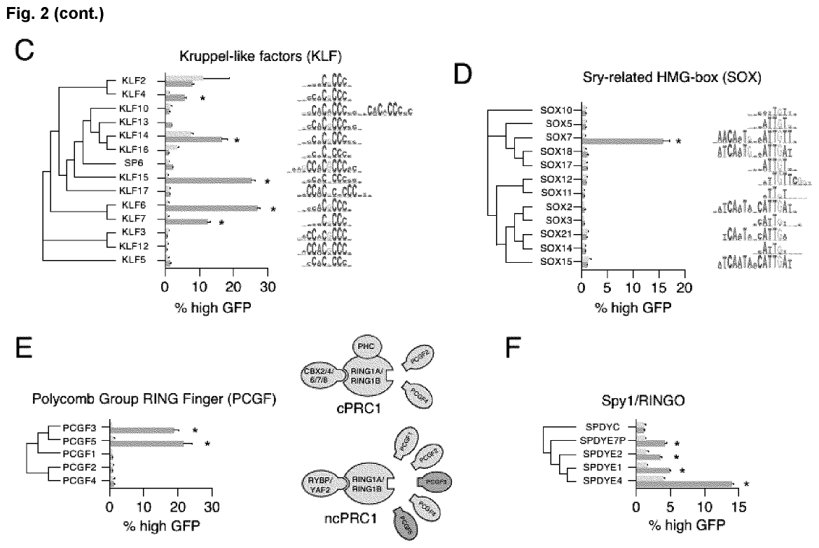

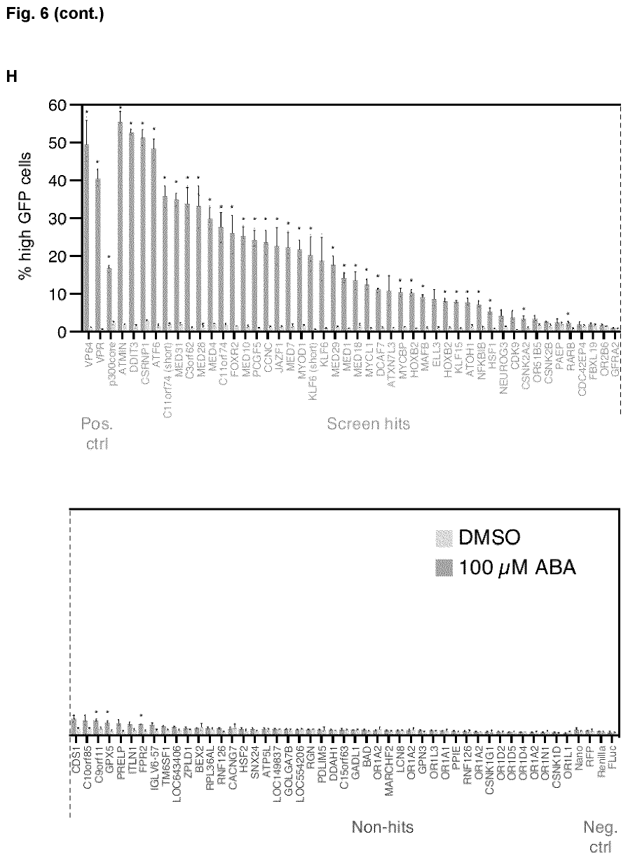

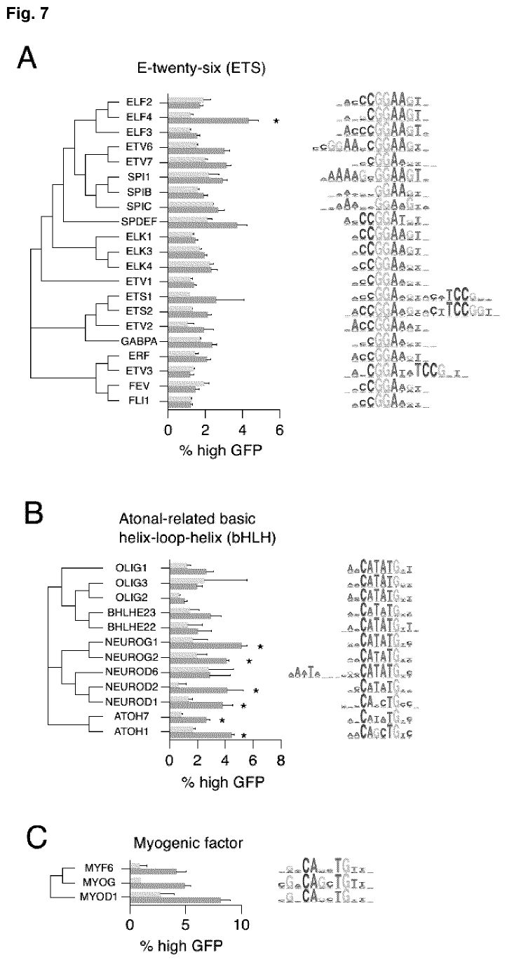

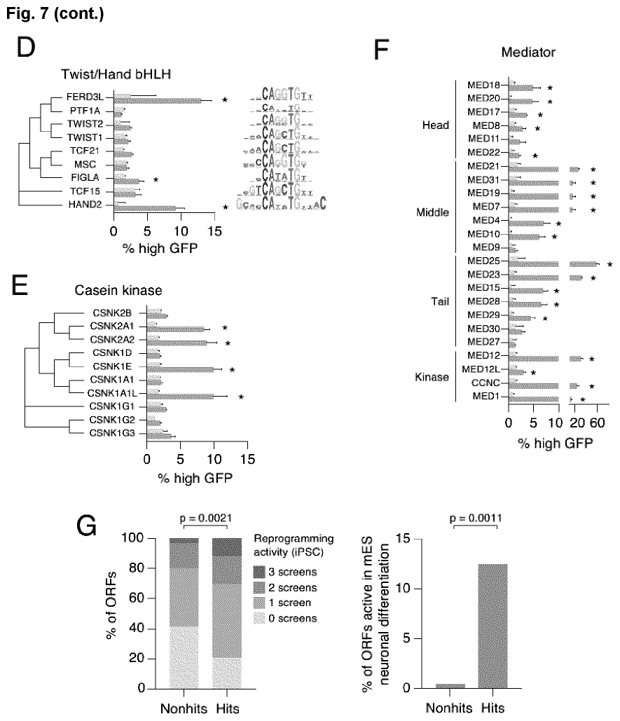

Transcription factors comprise multiple families, characterized by their distinct DNA-binding and auxiliary domains (Lambert et al., 2018). TFs that belong to the same family often have highly similar or even identical sequence specificities (Jolma et al., 2013; Lambert et al., 2018; Weirauch et al., 2014). Nevertheless, even highly related TFs can have distinct effects on transcription and chromatin due to their unique auxiliary domains. In line with this, only some members of transcription factor families were identified as hits in the pooled screen. To rule out sensitivity of the pooled approach as the cause, members of Forkhead-box (FOX), SRY-related HMG-box (SOX), E-twenty-six (ETS), atonal-related basic helix-loop-helix (bHLH), Twist/Hand, Kruppel-like factor (KLF), and Homeobox protein (HOX) families were individually assayed (FIGS. 2A and 7A). Two protein families linked to transcription and chromatin (Polycomb group RING finger (PCGF) and casein kinase family), one family (Spy1/RINGO) not previously linked to transcription (Gastwirt et al., 2007), and 24 Mediator subunits, which are not evolutionarily related but are integral components of the same, conserved complex were also included.

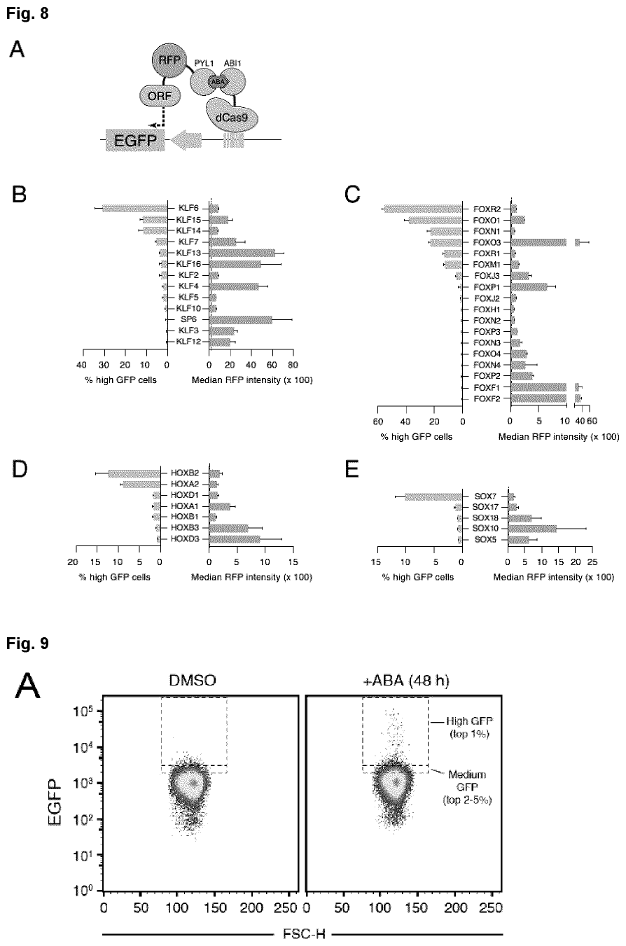

Consistent with the pooled screen, activation profiles for many highly homologous transcription factors were markedly different even when tested one at a time. For example, only five of the 37 Forkhead TFs, one of the 14 SOXs, five of 14 KLFs, and two of the 36 HOX proteins activated reporter expression (FIGS. 2A-2D and 7). To exclude the trivial explanation that the difference in activity was due to expression, several TFs were assayed as RFP-PYL1 fusions to account for fusion protein expression levels. The results were highly consistent with PYL1-only fusions and did not correlate with RFP levels (FIG. 8), indicating that the differences in transcriptional activity reflected the intrinsic potential of the TFs.

Notably, activating TFs identified in the screen were significantly enriched for factors that can induce differentiation of mouse embryonic stem cells and human induced pluripotent cells (iPSCs) when ectopically expressed (FIG. 7G)(Ng et al., 2021; Theodorou et al., 2009), suggesting that activation differences in the assay are related to differences in biological function. For example, of the related bHLH family transcription factors, NEUROG1, NEUROG2, NEUROD1, and NEUROD2 can induce neuronal differentiation of iPSCs whereas NEUROD6 cannot (Goparaju et al., 2017). This pattern is concordant with their ability to activate the reporter gene (FIG. 7B). Similarly, only four HOX factors (HOXA1, HOXA2, HOXB1, HOXB2) can activate the b1-ARE autoregulatory element located in the Hoxb1 locus (Di Rocco et al., 1997), and three of these were characterized as activators in the assay (HOXA2 and HOXB2 in the pooled screen and the arrayed assay, HOXA1 in the screen) (FIG. 2A). Moreover, recent work revealed a striking collinearity between the repressive potential, expression pattern, and genomic location of HOX genes (Tycko et al., 2020), with HOX transcription factors in the 5′ end of homeobox clusters being repressive. Consistent with this, the HOX family activators in the assay are encoded by the most or the next-to-last 3′ genes in their HOX gene clusters.

A particularly interesting case was that of PCGF family proteins, which are mutually exclusive components of canonical and non-canonical Polycomb Repressive Complexes 1 (PRC1) (Gahan et al., 2020; Gao et al., 2012)(FIG. 2E). Although generally thought to act in chromatin compaction and gene silencing in the context of PRC1, PCGF3 was identified as an activator in the original screen. PCGF3 robustly activated the reporter when tested individually, as did PCGF5 (which was not present in the original pooled screen) (FIG. 2E). In contrast, three other PCGF family members (PCGF1, PCGF2, and PCGF4/BM11) were neither screen hits nor activated the reporter when individually tested (FIG. 2E). PCGF5 has been previously shown to regulate transcriptional activation (Gao et al., 2014), but no such role has been described for PCGF3. Interestingly, in the PCGF family phylogeny, PCGF3 and PCGF5 form a distinct group that arose early during animal evolution (Gahan et al., 2020), suggesting that their transcriptional activation function is of ancient origin.

Some studies have indicated that subunits of the Mediator complex can have distinct regulatory functions, with some subunits promoting transcriptional activation and others promoting repression (Conaway and Conaway, 2011; Stampfel et al., 2015). In the assay, 20 of the 24 assayed Mediator subunits robustly activated the reporter, with no difference between Mediator submodules (FIG. 7F). For example, MED29 has been suggested to have transcriptional repressor activity (Wang et al., 2004), but it was both a hit in the pooled screen and validated as an activator when tested individually (FIG. 7F). Thus, at least in the context of the reporter system, almost all Mediator subunits promote transcriptional activation.

The primary activation screen identified two proteins (SPDYE4 and SPDYE7P) that belong to the Spy1/RINGO (Rapid INducer of G2/M progression in Oocytes) family of cell cycle regulators. Spy1/RINGO proteins bind to and activate Cdk1 and Cdk2 in a cyclin-independent manner, thereby promoting cell cycle progression (Gonzalez and Nebreda, 2020). However, they have not been previously implicated in transcriptional regulation. As a result of recent expansion (Chauhan et al., 2012), the human genome contains at least 19 Spy1/RINGO family genes and multiple pseudogenes. Five Spy1/RINGO proteins were individually tested for transcriptional activation, and four of them robustly activated the reporter (FIG. 2F). These results suggest that Spy1/RINGO proteins may promote cell cycle progression in part by functioning as transcriptional activators.

Methods are as described in Example 7.

### Example 4. TAD-Seq Reveals Novel Human Transactivation Domains

Transcription factors generally activate transcription through transactivation domains (TADs) that interact with co-activators, such as the Mediator, CBP/p300 acetyltransferases, or TFIID. Most TADs are short, unstructured sequences rich in acidic and hydrophobic residues (Sigler, 1988). Despite intensive efforts, no clear consensus motifs have emerged, making computational prediction of TADs challenging (Erijman et al., 2020; Ravarani et al., 2018; Staller et al., 2021). Several groups have recently implemented pooled recruitment approaches to identifying TADs from known transcriptional regulators or from random sequences (Arnold et al., 2018; Erijman et al., 2020; Ravarani et al., 2018; Sanborn et al., 2020). The screening platform described above was modified to identify the region(s) responsible for transcriptional activation among the hits. This approach was similar to the previously published TAD-seq method (Arnold et al., 2018) except that synthesized fragments were used instead of randomly fragmented DNA.

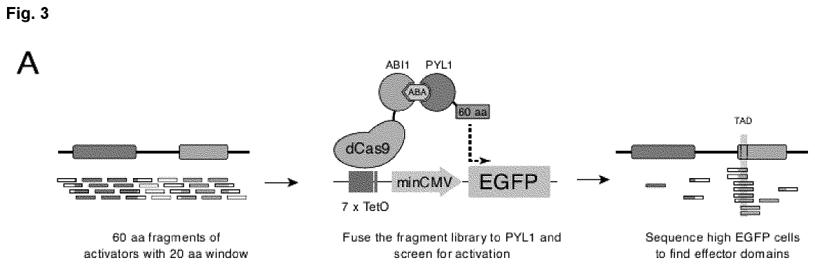

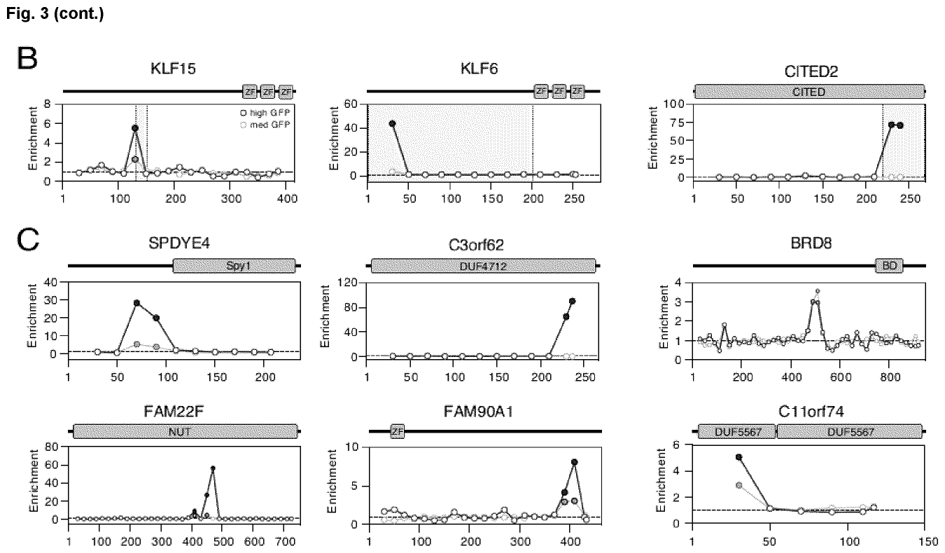

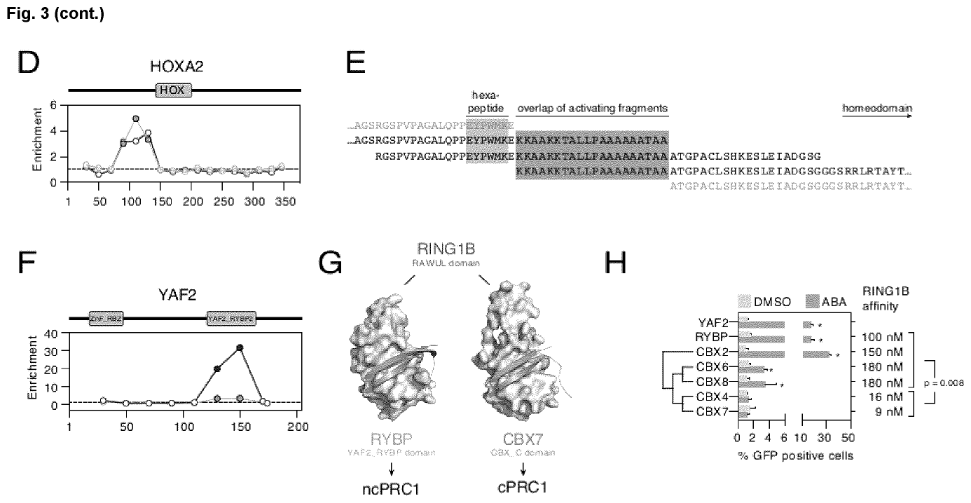

A fragment library of 75 activators identified in our screen was generated, using 60 amino-acid tiles every 20 aa such that every amino acid was represented by three different fragments (FIG. 3A). The pooled fragment library was fused to PYL1 and recruited to the same GFP reporter used in the original ORFeome screen (FIG. 3A). Again, ABA-dependent GFP expression was observed in a subset of reporter cells infected with the fragment library (FIG. 9A). The GFP positive population was sorted into two independent replicates, and fragments in each population were quantified by sequencing. However, in contrast to the ORFeome screen, both high GFP cells (top 1%) and medium GFP cells (top 2-5%) were sorted to potentially identify transactivators of different strength.

The pooled approach revealed 70 activating fragments in 39 different proteins. As expected, these fragments were enriched in acidic and hydrophobic amino acids, and depleted of positively charged (basic) amino acids (FIG. 9B), indicating that many represent “canonical” TADs consistent with the “acid blob” idea (Sigler, 1988). Indeed, activating fragments contained more predicted TADs based on two different algorithms (Erijman et al., 2020; Piskacek et al., 2007)(FIG. 9C) and the predicted TADs were longer in activating fragments than in inactive fragments (FIG. 9D). However, many active fragments were not predicted by any algorithm, highlighting the need for experimental approaches.

Of the 70 active fragments, the majority (44=63%) was identified in only the high GFP or the medium GFP population, suggesting that the fragments have distinct activation potential (FIG. 9E). For example, VP64 was enriched in the high GFP population but depleted in the medium GFP population when compared to the unsorted fragment library (FIG. 9E). This is consistent with the unusual potency of VP16 in transcriptional activation (VP64 consists of four VP16 peptides in tandem)(Sadowski et al., 1988). 10 fragments identified in the screen were individually tested, and 9 of them robustly activated the reporter (FIG. 9F), suggesting low false positive rate.

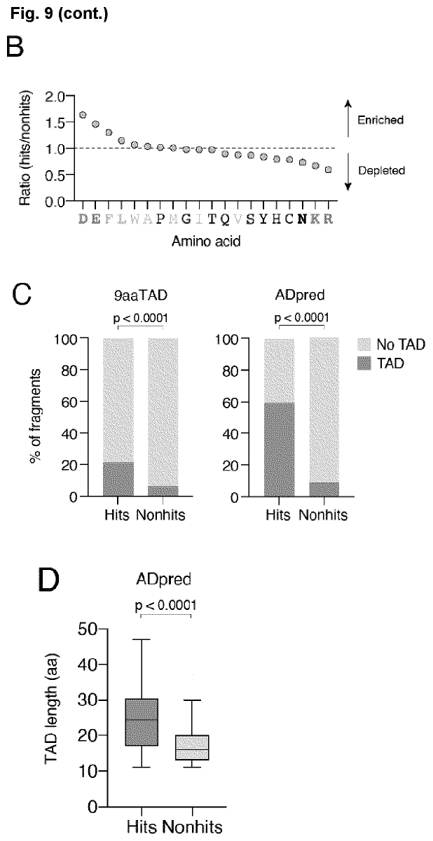

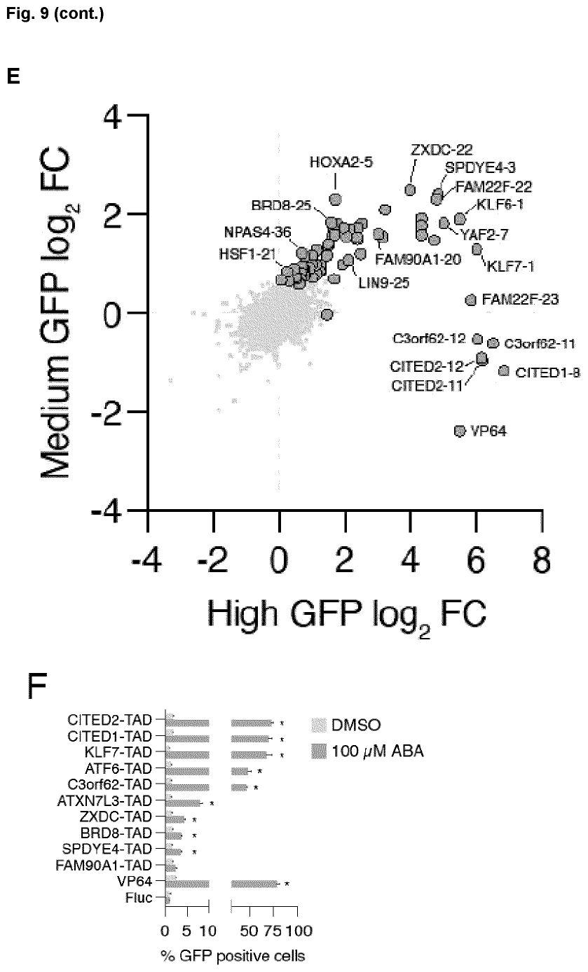

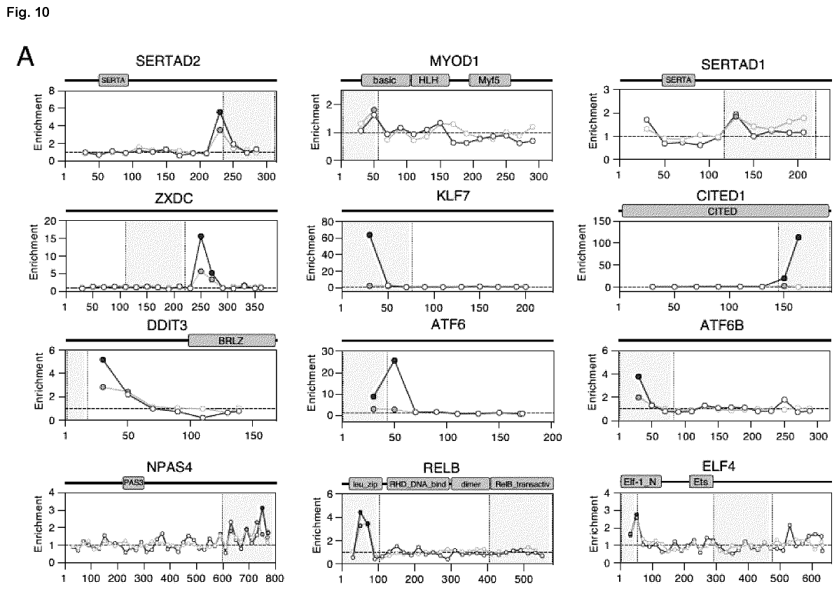

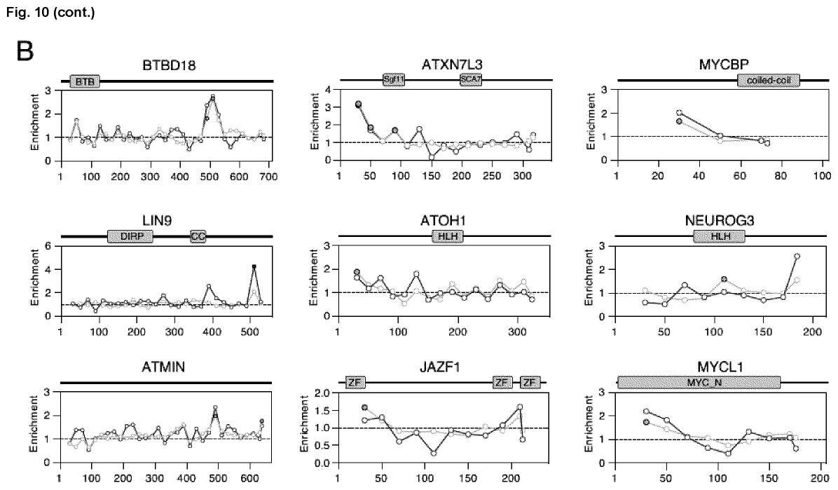

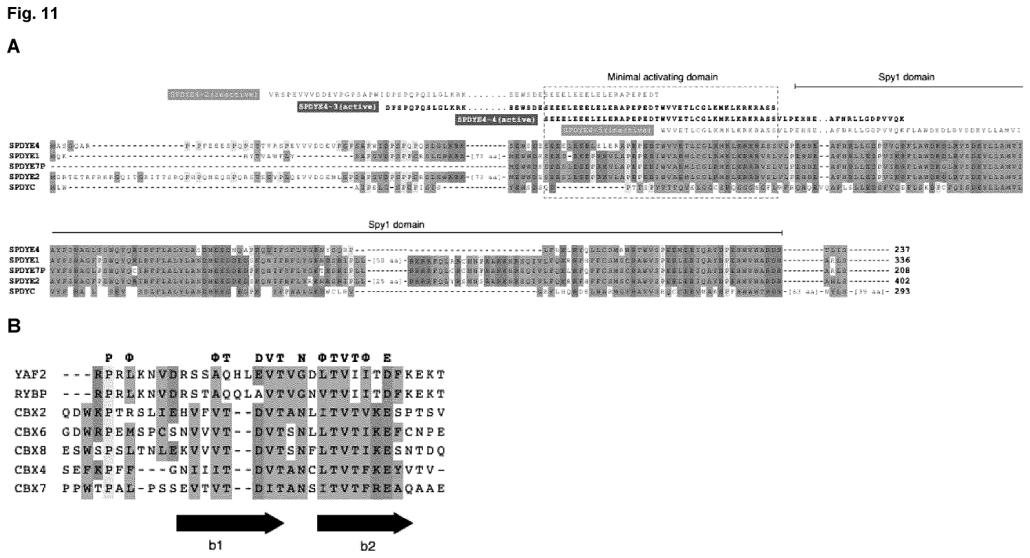

The fragment screen recovered several known TADs. For example, known TADs in KLF6, KLF7, KLF15, ATF6, and CITED2 were identified (FIGS. 3B and 10A). Additionally, since the overlap between active fragments can pinpoint the minimum sequence required for activity, the regions required for activity of previously identified TADs, such as those for KLF6, KLF7, and SERTAD2 were narrowed (FIGS. 3B and 10A). Many previously unknown TADs in both canonical transcriptional regulators and novel proteins were discovered in the ORFeome-wide screen (FIGS. 3C and 10B). TADs were uncovered in previously uncharacterized proteins SPDYE4, C3orf62, FAM22F, and FAM90A1 (FIG. 3C). Interestingly, the novel TAD in SPDYE4 was adjacent to its Spy1 domain that interacts with and activates Cdk2, indicating that the potential transcriptional activity and Cdk-regulating activity are mediated by distinct domains of Speedy family proteins (FIGS. 3C and 11A)(McGrath et al., 2017). Moreover, consistent with the transcriptional activation profiles of Spy1/RINGO family proteins, the TAD region is conserved in all family members except SPDYC, the only Spy1/RINGO family protein that was inactive in the recruitment assay (FIGS. 2F and 11A).

Interestingly, some of the uncovered TADs did not have characteristics of typical transactivation domains. For example, the three overlapping fragments in HOXA2 that activated transcription spanned a polyalanine stretch between the homeobox DNA-binding domain and the antennapedia-like hexapeptide motif (FIGS. 3D and 3E), which interacts with the PBX1 co-activator (Piper et al., 1999). However, a fragment lacking the hexapeptide motif still activated transcription, suggesting that the activity is not regulated via PBX1 (FIG. 3E). Polyalanine stretches are functionally important in Ultrabithorax (Ubx) proteins in Drosophila, and recently they were suggested to have a role in driving phase separation of many TFs such as HOXD13 (Basu et al., 2020). The results herein suggest that polyalanine stretches may have additional roles in transcriptional activation.

Another non-canonical activating region was from YAF2, which is a component of the Polycomb Repressive Complex 1 (PRC1) (Gao et al., 2012) and a prominent hit in the original ORFeome screen (FIG. 3F). This region contained the YAF2_RYBP domain, which folds into an antiparallel beta sheet that binds RING1B, a core PRC1 subunit (Wang et al., 2010) (FIG. 3G). The YAF2_RYBP domain of YAF2 and its close homolog RYBP shares sequence and structural homology with the CBX-C domain present in the CBX family Polycomb proteins (Wang et al., 2010)(FIG. 11B). CBX-C domain containing proteins and YAF2/RYBP interact in a mutually exclusive manner with RING1A and RING1B to form canonical (cPRC1) and non-canonical (ncPRC1) Polycomb complexes (FIG. 3G).

To test if all CBX-C and YAF2_RYBP domains can promote transcriptional activation, the YAF2_RYBP domains of YAF2 (SEQ ID NO: 96) and RYBP (SEQ ID NO: 140) and the CBX-C domains of CBX2 (SEQ ID NO: 136), CBX4, CBX6 (SEQ ID NO: 138), CBX7, and CBX8 (SEQ ID NO: 139) were cloned and assayed for activity with the reporter system. The YAF2_RYBP motif from both YAF2 and RYBP robustly activated the reporter, consistent with the TADseq results for YAF2 (FIG. 3H). Some CBX-C domains were also activators: CBX-C from CBX2 was the most potent activator, whereas those from CBX6 and CBX8 activated the reporter weakly (FIG. 3H). In contrast, CBX4 and CBX7 CBX-C domains had no effect on the reporter. This pattern reflected the evolutionary ancestry of the CBX-C domain proteins (FIG. 3H). Interestingly, the CBX-C domains of CBX4 and CBX7 bind RING1B with ˜10-fold higher affinity than the same domains from CBX proteins that activate transcription, or the YAF2_RYBP domain of RYBP (p=0.008, two-tailed t-test)(FIG. 3H)(Wang et al., 2008, 2010). Thus, the differences in the transcriptional activity of CBX-C and YAF2_RYBP domains might be explained by their binding affinity for RING1B or by differential binding to other factors. In any case, these results suggest that the differences in CBX protein function (Morey et al., 2012; Vincenz and Kerppola, 2008) could be at least partially explained by the intrinsic differences in their CBX-C domains. More broadly, these results reveal another layer of complexity in the assembly and function of noncanonical and canonical PRC2 complexes.

Methods are as described in Example 7.

### Example 5. Novel Transcriptional Activators Interact with Known Co-Factors

The ORFeome-wide screen revealed several potent transactivators that were either poorly or completely uncharacterized. To understand how these factors regulate transcription, stable, tetracycline-inducible HEK293 cell lines were established, expressing nine poorly characterized screen hits (C3orf62, C11orf74/IFTAP, NCKIPSD, DCAF7, SS18L2, SPDYE4, FAM90A1, FAM22F/NUTM2F, JAZF1), five known transcriptional regulator hits (SOX7, KLF6, KLF15, CTBP1, HOXA2, and HOXB2), two synthetic transactivators (VP64 and VPR), and negative controls (EGFP and Nanoluc) fused to biotin ligase BirA from Aquifex aeolicus and FLAG epitope tag. Their protein interactomes were characterized with affinity-purification coupled to mass spectrometry (AP-MS) and proximity partners with proximity-dependent biotinylation (BiolD2)(Kim et al., 2016). AP-MS is an ideal method for characterizing stable protein complexes, whereas BiolD excels in identifying interactions that are weaker or involve poorly soluble proteins, such as those tightly bound to chromatin (Lambert et al., 2015).

The interactomes of transcriptional activators revealed two patterns. First, they indicated that the transactivation potential of the novel hits likely reflects their endogenous function rather than being an artefact of the tethering assay. Second, the interactomes uncovered a striking preference of the activators for specific co-activator complexes, converging on five distinct co-factors (CBP/p300, BAF, NuA4, Mediator, and TFIID).

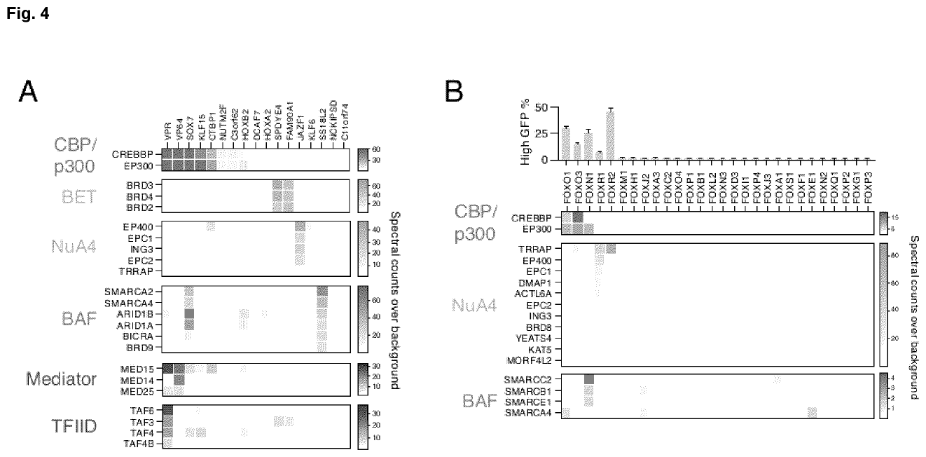

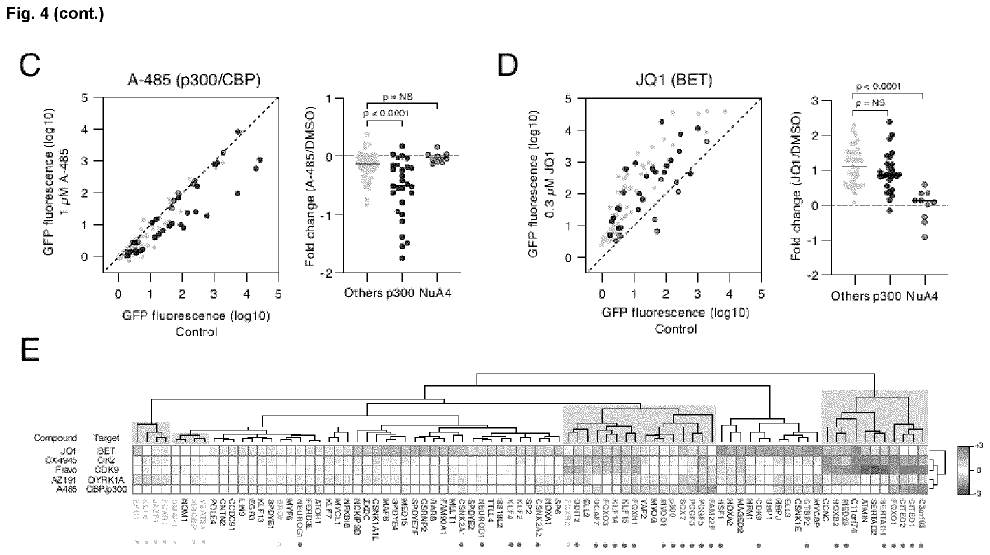

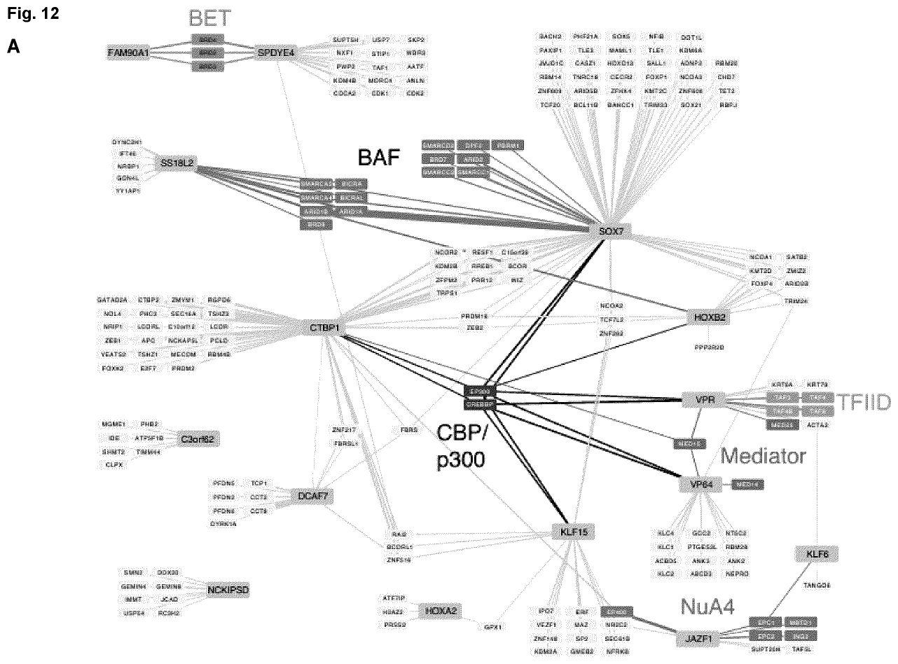

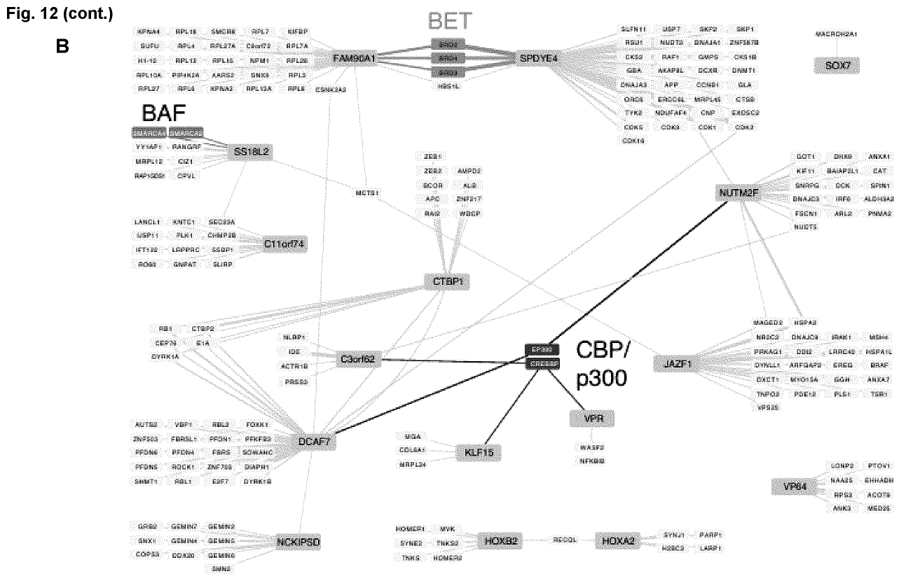

Supporting a native role for the novel activators in transcriptional regulation, eight of the nine poorly characterized hits associated with known transcriptional co-factors in AP-MS, BiolD, or both (FIG. 4A, FIG. 12). C3orf62, DCAF7, and FAM22F/NUTM2F interacted with p300 and/or CBP, which are known transcriptional co-activators. JAZF1, in contrast, associated with multiple subunits the NuA4 histone acetyltransferase complex in BiolD, suggesting that it is a novel subunit of this highly conserved complex. SS18L2, in turn, interacted with the BAF chromatin remodeling complex, including core BAF members SMARCA2 and SMARCA4 as well as subunits specific to the canonical BAF (cBAF) and non-canonical BAF (ncBAF) (FIG. 4A and 12)(Centore et al., 2020). SPDYE4 and FAM90A1 interacted with the BET family bromodomain proteins BRD2, BRD3, and BRD4, which regulate transcriptional elongation (Fujisawa and Filippakopoulos, 2017). NCKIPSD, also known as SPIN90, interacted with the survival of motor neurons (SMN) complex, which regulates the assembly of ribonucleoprotein complexes but has also been linked to transcriptional activation (Pellizzoni et al., 2001; Singh et al., 2017; Strasswimmer et al., 1999) (FIG. 12). Interestingly, NCKIPSD also interacted with DCAF7 (FIG. 12B). Both DCAF7 and NCKIPSD have been implicated in the regulation of actin dynamics in the cytoplasm (Cao et al., 2020; Morita et al., 2006), suggesting that these proteins may have distinct roles in the cytoplasm and in the nucleus.

Known transcriptional regulators also associated with co-activator complexes (FIGS. 4A and 12). KLF6 bait identified the NuA4 subunit EPC1 as a proximity interactor, whereas HOXB2, KLF15, CTBP1, and SOX7 baits had CBP and p300 as proximity partners. In addition, SOX7 also associated with BAF subunits. In contrast to all native activators except for SOX7, the powerful synthetic activators VP64 and VPR identified multiple different co-activators as proximity partners. VP64, which consists of four tandem copies of the viral VP16 transactivation motif, associated with CBP/p300 and mediator subunits MED14 and MED15, consistent with previous reports (Kundu et al., 2000; Yang et al., 2004). VPR, which is a fusion of VP64, human p65/RELA transactivation domain, and Epstein-Barr virus R transactivator (Chavez et al., 2015), associated with even more co-factors, including CBP/p300 and multiple subunits of the Mediator and TFIID (FIGS. 4A and 12A). Such association with multiple co-factors likely explains the exceptional transactivation potency of VPR when fused to dCas9 (Chavez et al., 2015, 2016).

These results suggest that activating transcription factors and other transcriptional regulators have a strong intrinsic preference for specific co-factors. To further investigate this, a previously published AP-MS interaction dataset of Forkhead family TFs (Li et al., 2015) was analyzed and compared with the transcriptional activation results in this assay. Notably, only those Forkhead TFs that activated transcription when recruited to the reporter interacted with co-activators in AP-MS (FIG. 4B). In addition, similar to the mass spectrometry results, activating Forkhead TFs had distinct co-factor preferences: FOXO1 and FOXO3 interacted specifically with CBP and p300, FOXN1 interacted with both p300 and subunits of the BAF complex, whereas FOXR1 and FOXR2 preferred the NuA4 complex (FIG. 4B). These data strongly suggest that related transcription factors, which recognize highly similar sequences and activate transcription nearly to the same extent, can promote transcription through distinct co-activator complexes.

To functionally investigate the connection between transcriptional activators and co-factors, a panel of 83 robust activators was arrayed. Their activation potential was tested using the reporter assay, but now in the presence of small-molecule inhibitors targeting multiple transcriptional co-regulators. Three kinase inhibitors targeting transcriptional kinases (flavopiridol for CDK9, CX-4945 for casein kinase 2, and AZ191 for DYRK1A and DYRK1B), and two compounds inhibiting transcriptional co-factors (A-485 for CBP/p300, and JQ1 for BET family bromodomain proteins) were employed.

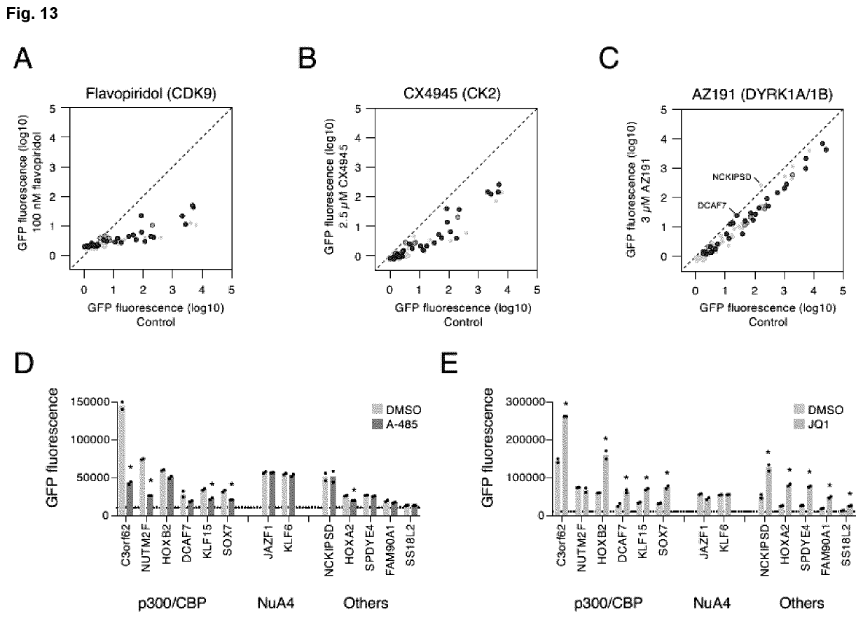

The three kinase inhibitors affected nearly all activators, although to a different degree. Inhibiting CDK9 with flavopiridol led to nearly complete loss of activity of all activators, consistent with the key role of p-TEFb in promoter clearance (FIG. 13A). Inhibition of casein kinase 2, which regulates transcriptional elongation (Basnet et al., 2014), also led to a general albeit modest attenuation of transcriptional activation (FIG. 13B). DYRK1A/DYRK1B inhibition had a more subtle effect than the other two compounds, slightly attenuating the activity of most activators (FIG. 13C). Interestingly, two activators that were not affected by the DYRK1A/DYRK1B inhibitor AZ191 were DCAF7, which forms a conserved complex with DYRK1A (Breitkreutz et al., 2010; Yu et al., 2019), and NCKIPSD, which interacts with DCAF7 (FIG. 13C).

In contrast to kinase inhibitors that had broad effects on transcription, inhibiting the acetyltransferase activity of CBP/p300 strongly affected the activity of some but not all transactivators (FIG. 4C). Importantly, these effects were consistent our AP-MS and BiolD results: the activity of four of the six CBP/p300 interactors was significantly decreased, whereas only one of the seven non-interactors was affected by A-485 (FIG. 13D). For example, A-485 inhibited the activity of KLF15, whereas it had no effect on the related Kruppel-like factor KLF6 (FIG. 13D). More broadly, the activity of proteins known to interact with CBP/p300 was significantly more inhibited by A-485 than that of non-interacting transactivators (FIG. 4C). In comparison, interactors of NuA4 complex were not significantly affected by CBP/p300 inhibition (FIGS. 4D and 13D).

Similar to CBP/p300 inhibition, BET family inhibition with JQ1 had distinct effects on some transactivators. Interestingly, in most cases JQ1 treatment led to an increase in reporter gene activity (FIG. 4D). Although JQ1 treatment leads to rapid downregulation of BRD4 target genes in many cases (Loven et al., 2013; Muhar et al., 2018), it has also been linked to transcriptional activation of reporter genes (Sdelci et al., 2016), which likely explains the effect observed. Nevertheless, not all transactivators responded similarly to JQ1 treatment. In particular, factors interacting with subunits of NuA4 complex were not affected by JQ1 treatment (e.g., JAZF1 and KLF6; FIGS. 4D and 13D). Indeed, NuA4 interactors were significantly less affected by JQ1 treatment that CBP/p300 interactors or other transactivators (FIG. 4D). Although the mechanism by which NuA4 interactors respond to JQ1 treatment in a unique manner requires further research, these results demonstrate how transcriptional activators promote transcription via different co-activator complexes in the context of a single promoter. Indeed, hierarchical clustering of the activators based on their sensitivity to the five compounds revealed multiple distinct groups (FIG. 4E). Many paralogous factors (such as CITED1 and CITED2, or PCGF3 and PCGF5) clustered next to each other, indicating that clustering produced functionally relevant groups. Moreover, known CBP/p300 interactors were primarily in two distinct clusters, as were NuA4 interactors (FIG. 4E). It is likely that other members of these clusters similarly use CBP/p300 or NuA4 as co-activators, such as YAF2 or MYOG for p300, or NOM1 for NuA4.

Methods are as described in Example 7.

### Example 6. SRF-C3orf62 Fusion Interacts with CBP/p300 and Promotes SRF/MRTF Transcriptional Program

Fusion proteins involving transcriptional regulators are common hallmarks of certain cancers, such as leukemias and sarcomas. Hits from our ORFeome-wide screen were significantly enriched for genes documented in the COSMIC database (cancer.sanger.ac.uk) as fusion partners in diverse cancers (p=0.019; hypergeometric distribution test). These included well-characterized fusion partners such as ERG, which is fused to EWSR1 in Ewing sarcoma and to TMPRSS2 in prostate cancer; DDIT3/CHOP, fused to EWSR1 or FUS in myxoid liposarcoma; CRTC1, fused to MAML2 in mucoepidermoid carcinoma; and ENL/MLLT1, fused to MLL in mixed lineage leukemia. In addition, several hits have been described in literature as fusion partners but not functionally characterized. For example, BTBD18 and NCKIPSD were identified as KMT2A/MLL fusion partners leukemia (Alonso et al., 2010; Sano et al., 2000). Moreover, the fragment of BTBD18 that is fused to MLL contains the transactivation domain identified by TAD-seq (FIG. 10B), implicating the TAD in the oncogenic potential of the fusion product. Most MLL fusions involve genes regulating transcriptional elongation, such as the super elongator complex (Winters and Bernt, 2017). Interestingly, BTBD18 has also been shown to promote transcriptional elongation (Zhou et al., 2017).

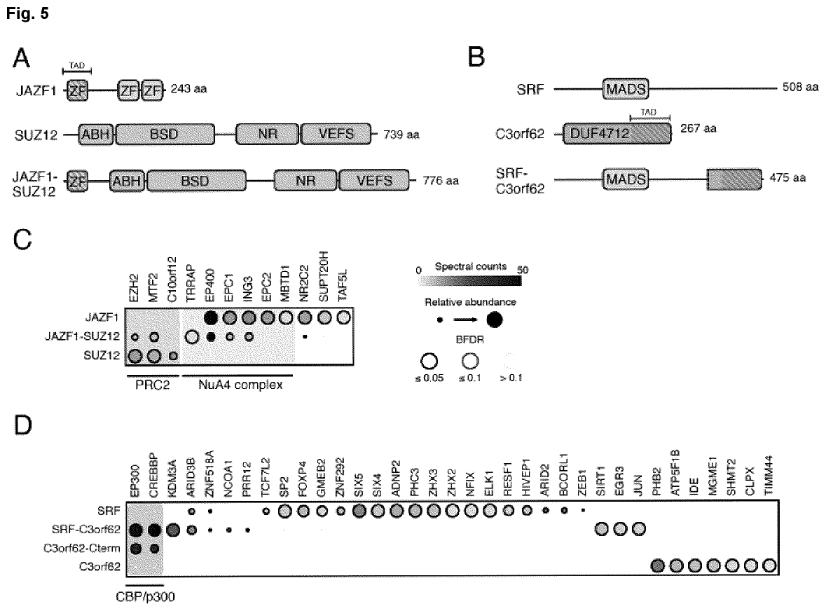

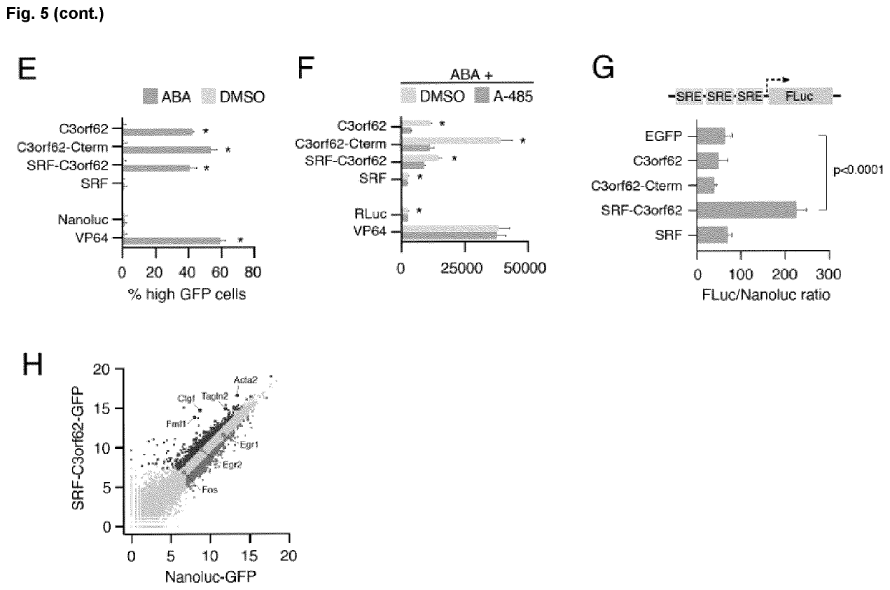

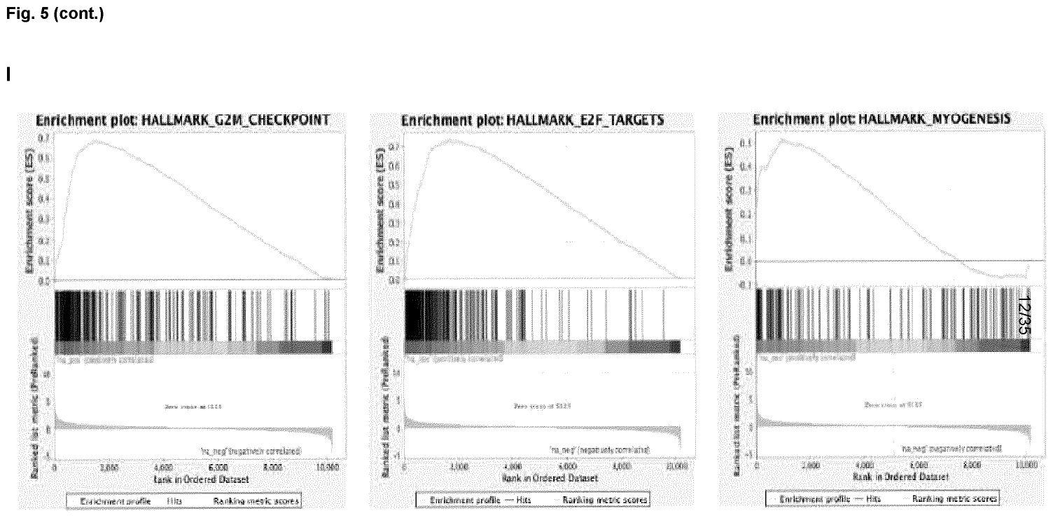

To gain more insight into the mechanisms by which the activation screen hits might promote tumorigenesis as fusion partners, two poorly characterized fusions, JAZF1-SUZ12 and SRF-C3orf62 were selected for further characterization. The JAZF1-SUZ12 fusion is a hallmark of low-grade endometrial stromal sarcoma (LG-ESS)(Hrzenjak, 2016), bringing together the Polycomb protein SUZ12 and JAZF1 (FIG. 5A)(Piunti et al., 2019). SRF-C3orf62 was recently described in a pediatric case of myofibroma/myopericytoma (Antonescu et al., 2017). In this fusion, the DNA-binding domain of Serum Response Factor (SRF) is fused to the C-terminus of C3orf62 (FIG. 5B). Notably, in both cases the transactivation domain that identified by TAD-seq (FIGS. 3C and 10B) is retained in the fusion constructs (FIGS. 5A and 5B)

SUZ12, SRF, JAZF1-SUZ12, SRF-C3orf62, and the C-terminal fragment of C3orf62 fused to SRF (C3orf62-Cterm) were tagged with BirA-FLAG and their interactomes were analyzed with BiolD and AP-MS, to complement the data we obtained for JAZF1 and C3orf62. As expected, SUZ12 proximity partners included other components of the PRC2 complex, such as EZH2, MTF2/PCL2 and C10orf12 (Alekseyenko et al., 2014)(FIG. 5C). Strikingly, the JAZF1-SUZ12 fusion protein had both PRC2 and NuA4 subunits as proximity partners (FIG. 5C), indicating that this fusion assembles into a supercomplex of two chromatin-associated complexes that are normally associated with opposing transcriptional activities. Consistent with this, EPC1-PHF1 fusion that is also associated with LG-ESS similarly assembles the PRC2-NuA4 supercomplex, which leads to aberrant expression of Polycomb targets (Sudarshan et al., 2021). Moreover, both JAZF1-SUZ12 and EPC1-PHF1 were recently shown to be strong transcriptional activators (Sudarshan et al., 2021). Thus, NuA4 integration into the oncogenic supercomplex can override the normally repressive function of PRC2 complexes.

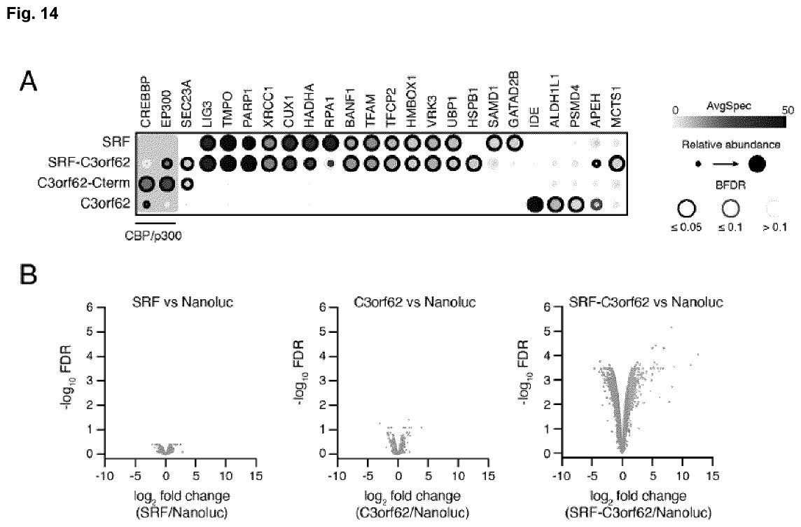

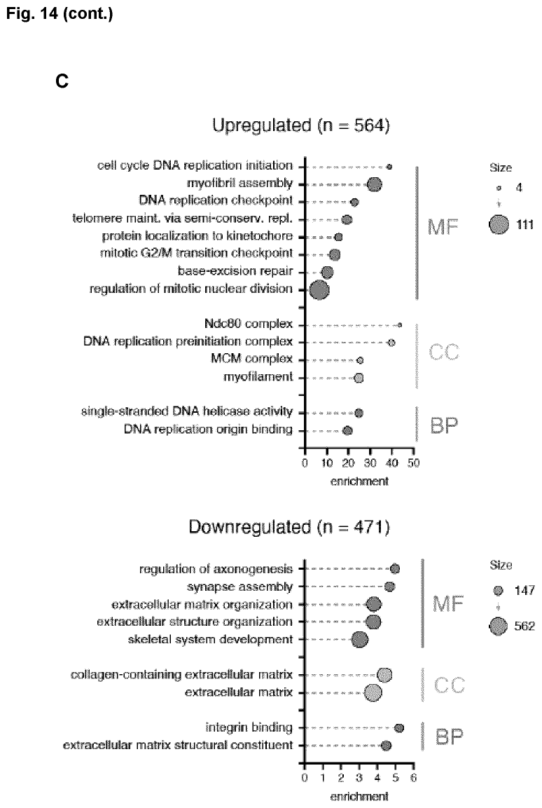

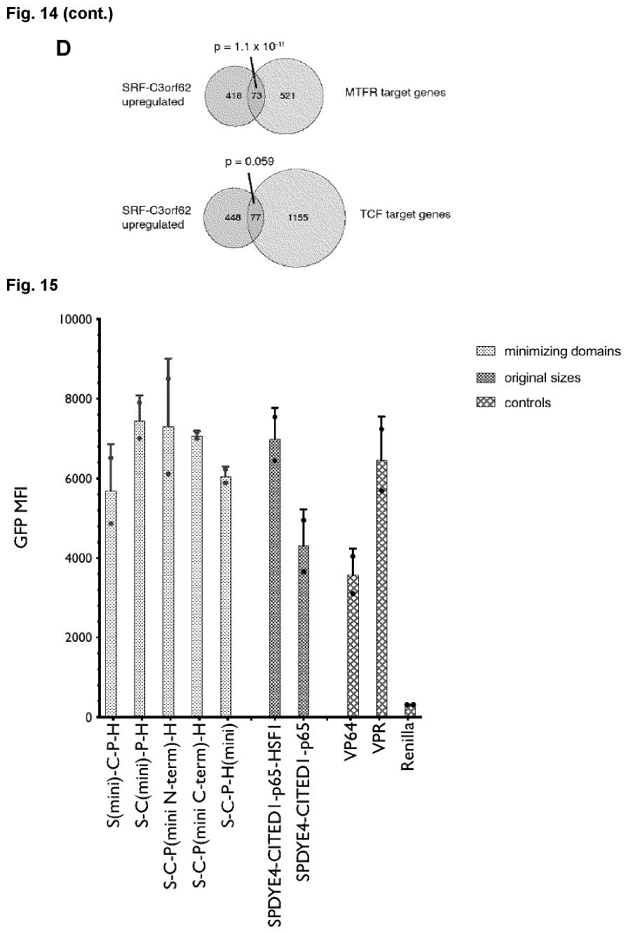

SRF proximity partners included multiple transcription factors such as ELK1, which forms a ternary complex with SRF on serum response elements (SREs)(Buchwalter et al., 2004)(FIG. 5D). However, it did not associate with any transcriptional co-activators. In contrast, both C3orf62-Cterm construct and the SRF-C3orf62 fusion robustly identified both CBP and p300 as proximity partners (FIG. 5D). AP-MS similarly identified CBP and p300 as prominent SRF-C3orf62 interactors (FIG. 14A). Consistent with these results, both C3orf62-Cterm and SRF-C3orf62 strongly activated the GFP reporter when tethered to the reporter (FIG. 5E). This activity was highly sensitive to CBP/p300 inhibition with A-485 (FIG. 5F), in line with the interaction patterns of SRF-C3orf62.

SRF functions together with either ternary complex factors (TCFs; e.g. ELK1) or myocardin-related transcription factors (MRTFs; e.g. MAL) to regulate target gene expression (Buchwalter et al., 2004; Olson and Nordheim, 2010). The results suggested that SRF-C3orf62 can activate SRF target genes without such cofactors. To test this further, the activity of different constructs was assayed in NIH3T3 fibroblasts using a luciferase-based serum response element reporter (Vartiainen et al., 2007). SRF or C3orf62 alone did not activate the reporter whereas SRF-C3orf62 robustly did so (FIG. 5G), further confirming that the cancer-associated fusion bypasses the requirement for SRF cofactors in transcriptional activation.

The SRF/TCF pathway, which is regulated by MAP kinase signaling, regulates the expression of immediate-early genes (Gualdrini et al., 2016), whereas the actin-Rho signaling dependent SRF/MRTF pathway targets genes involved in cell motility and adhesion (Miralles et al., 2003). To test if SRF-C3orf62 can regulate target genes of both pathways, stable doxycycline-inducible NIH3T3 cell lines stably expressing SRF, C3orf62, SRF-C3orf62 and Nanoluc fused to GFP were generated. Transgene expression was induced with doxycycline for 24 hours and changes in gene expression were analyzed by RNA-seq. While expression of GFP-tagged C3orf62 or SRF had very limited or no effects on the transcriptome compared to Nanoluc (FIG. 14B), expression of SRF-C3orf62-GFP led to upregulation of 564 genes and downregulation of 471 genes (with log 2 fold change >1, FDR <0.05; FIGS. 5H and 14B). Gene set enrichment analysis (GSEA) and Gene Ontology analysis revealed that the upregulated genes were highly enriched in genes involved in DNA replication, cell cycle progression, and mitosis (FIGS. 51 and 14C), whereas downregulated genes were related to extracellular matrix (FIG. 14C). Upregulated genes were also significantly enriched in targets of the E2F transcription factor, a key regulator of cell proliferation (FIG. 51). These signatures are consistent with the oncogenic nature of the SRF-C3orf62 fusion. In addition, many of the most upregulated genes were involved in actin dynamics and myogenesis. For example, one of the most upregulated genes was smooth muscle actin (ACTA2), a known target of SRF/MTRF and a hallmark of SRF fusion positive myofibromas/myopericytomas (Antonescu et al., 2017)(FIG. 5H). Consistent with this, another significant signature of the upregulated genes was myogenesis (FIG. 5I). In contrast, many well-characterized immediate-early target genes of SRF/TCF such as FOS, EGR1, or EGR2 were not affected by ectopic SRF-C3orf62 expression (FIG. 5H). Interestingly, fusion of SRF to VP16 can potently upregulate FOS, suggesting that there are intrinsic differences in the ability of different TADs to activate target gene expression (Schratt et al., 2002). Indeed, there was a significant overlap between genes upregulated by SRF-C3orf62 and previously reported target genes of SRF/MRTF, but not with those of SRF/TCF (FIG. 14D)(Esnault et al., 2014; Gualdrini et al., 2016). Taken together, these results indicate that SRF-C3orf62 expression leads a proliferative and myogenic gene expression signature and preferential expression of SRF/MRTF target genes over SRF/TCF targets.

Methods are as described in Example 7.

### Example 7. Methods

Cell culture. All HEK293T cells, including the pTRE3G-EGFP reporter cell line (gift from Lei Stanley Qi lab, Stanford University) used for screens, were maintained in DMEM with 10% fetal bovine serum (FBS). NIH-3T3 cells were obtained from Dr. Sachdev Sidhu's lab (University of Toronto) and maintained in Dulbecco's Modified Eagle's Medium (DMEM) with 10% bovine calf serum (BCS). All culture media were supplemented with 1% penicillin-streptomycin. Cells were maintained at 37° C. in a humidified incubator at 5% CO2 and routinely tested for mycoplasma contamination.

Lentivirus production. Lentiviral particles containing the pooled ORFeome and transactivation libraries were produced by transfecting 293T cells with pLX301-ORFs/TADs-PYL1, psPAX2 (Addgene #12260) and pVSV-G (Addgene #8454) at a ratio of 8:6:1. Transfection was performed using XtremeGENE 9 (Roche) on 15-cm dishes according to the manufacturer's protocol. The medium was changed 6-8 hours post-transfection to harvest medium (DMEM+1.1 g per 100 mL BSA). 72 hours after transfection, supernatant was filtered (0.45 μM), pooled and collected. A similar protocol was followed for small scale virus production when establishing individual stable cell lines with transfection being performed on 6-well plates using Lipofectamine 2000 (Thermo Fisher Scientific, 11668019) reagent.

Cell line generation. A clonal line of the EGFP reporter line was generated expressing ABI-dCas9 (blastacidin, 6 μg/mL) and gRNA (SEQ ID NO: 10) (co-expressing EBFP2) targeting the pTRE3G promoter. Single cells were sorted (FACS Aria Illu, BD) and expanded and a clone showing induction by a strong transcriptional activator was selected for subsequent experiments. To generate NIH3T3 cells expressing doxycycline-inducible EGFP tagged proteins, entry clones were picked from the hORFeome collection and subcloned into the Gateway compatible pSTV6-TetO-ccdB-EGFP lentiviral plasmid (a kind gift from Payman Samavarchi-Tehrani). NIH-3T3 cells were infected in the presence of 8 μg/mL polybrene and selected with 2 μg/mL puromycin 24 hours post infection.

Pooled ORFeome library generation. Entry clones from the human ORFeome collection (v8.1) were collected into 40 standardized subpools each containing ˜384 ORFs and cloned into the lentiviral Gateway-compatible destination vector pLX301-DEST-PYL1. LR reactions were set up in duplicates with 150 ng of each entry ORF subpool, combined with 1 μl of Gateway LR clonase II in a total of 5 μl reaction volume and incubated overnight in TE buffer at room temperature. For the next two days, 1 μl additional LR enzyme was added in 4 μl TE and 150 ng destination vector to each reaction. Colonies were transformed into chemically competent DH5alpha E. coli and spread on LB agar plates containing carbenicillin (100 μg/μl) overnight at 30° C. Colonies were counted to ensure >200-fold coverage, collected in SOC on ice, pelleted and maxiprepped on multiple columns based on weight of the dry pellets.

Activation domain tiling library generation. A tiling library was generated from 75 proteins identified as activators in the ORFeome screen. Oligonucleotides containing a 5′ adapter GGAAGTCAGGGTAGCGGAAGTATG (SEQ ID NO: 23) and a 3′ adapter GGAGGTAGTGTTGAACGCGAAGGC (SEQ ID NO: 24) to generate a 5′ adapter (GSQGSGSM) (SEQ ID NO: 11) and a 3′ adapter (GGSVEREG) (SEQ ID NO: 12) were synthesized as pooled libraries (Twist Biosciences). 6×50 μl PCR reactions were set up using NEBNext Ultra II Q5 master mix (New England Biolabs) with 5 nM oligos as template. PCR conditions were optimized to find the lowest cycle with a clean visible product at the expected 300 bp length. The thermocycling condition was an initial 30 s at 98° C., then 2 cycles of 98° C. for 10 s, 63° C. for 20 s, and 72° C. for 15 s, followed by 10 more cycles of 98° C. for 10 s and 72° C. for 30 s with a final extension at 72° C. for 5 min. Primers were designed to have Gateway compatible flanking sequences. The resulting libraries were gel extracted by QIAgen gel extraction kit after loading on a 2% TAE gel for 2 hrs at 60V and subsequently cloned into pDONR221 using 20 separate BP reactions in total of 5 μl reactions. The entry plasmid pool was transformed after an overnight reaction into DHSalpha competent E. coli and incubated overnight on LB agar plates containing kanamycin (100 μg/μl). Colonies were collected and plasmid DNA purified. 20 LR reactions were then set up as described in the previous section. Each reaction was transformed into NEB 10-beta chemically competent E. coli and grown on LB agar plates containing carbenicillin (100 μg/μl) overnight at 30° C. Colonies were counted to ensure >200-fold coverage at each step of cloning, pooled and maxiprepped.

Pooled activation screens. ORFeome and transactivation tiling libraries tagged at the C-terminus with PYL1 were packaged into lentiviral particles. A clonal EGFP reporter cell line stably co-expressing ABI-dCas9 and a gRNA targeting the promoter were transduced at low multiplicity of infection (MOI) with approximately 30% cell survival after puromycin (1 μg/mL) selection. Untransduced cells under the same condition were fully eliminated. Sufficient cells were transduced to maintain >500 fold coverage of the libraries. Recruitment was induced by treating cells with 100 μM abscisic acid (ABA, Sigma) for 48 hours. In parallel, a control batch of cells were treated with equal total volume of DMSO. Cells were then washed in PBS, treated with dissociation buffer (1 mM EDTA, 10 mM KCl, 150 mM NaCl, 5 mM sodium bicarbonate, 0.1% glucose) and resuspended in flow buffer (5 mM EDTA, 25 mM HEPES pH 7, 1% BSA, PBS). High GFP population for each library, top 1% for ORFeome and two bins of top 1% and the next 4% for the TAD screen, were sorted and their genomic DNA directly extracted using QIAmp DNA Blood Mini Kit (QIAGEN).

ORFeome sequencing. Nested PCR was performed using all the purified genomic DNA from sorted populations or at least 5 μg of genomic DNA from presort populations. The target ORFeome region was amplified from genomic DNA using primers targeting the T7 promoter and PYL1. The product of this reaction was pooled for each sample and further amplified by primers targeting outside the Gateway attB sites for an additional 10 cycles. Amplicons were subsequently separated on 1% agarose gel and any visible PCR product excluding primer dimers were gel purified. After quantifying DNA using the Quant-iT 1× dsDNA HS kit (Thermo Fisher Scientific, Q33232), 50 ng per sample was processed using the Illumina DNA Prep, (M) Tagmentation kit (Illumina, 20018705), with 6 cycles of amplification. 2 μl of each purified final library was run on an Agilent TapeStation HS D1000 ScreenTape (Agilent Technologies, 5067-5584). The libraries were quantified using the Quant-iT 1× dsDNA HS kit (Thermo Fisher Scientific, Q33232) and pooled at equimolar ratios after size-adjustment. The final pool was quantified using NEBNext Library Quant Kit for Illumina (New England Biolabs, E7630L) and paired-end sequenced on an Illumina MiSeq.

TAD sequencing. Performed nested PCR on the purified genomic DNA using primers targeting T7 promoter and PYL1 of the backbone vector in the first step creating a ˜470 bp product. Products of the first reaction were then pooled and amplified for an additional 10 steps using primers targeting outside the Gateway sites creating a ˜300 bp product. Libraries were quantified on Qubit dsDNA Broad Range kit and paired-end sequenced on an Illumina MIseq with a custom PAGE-purified R1 sequencing primer.

Analysis of sequencing data from pooled activation screens. An index of the ORFeome reference sequences was created using the STAR aligner v2.7.8a with the length of the pre-indexing string set to 11 to account for the smaller ‘genome’ size. Reads from the ORFeome libraries were aligned with the STAR aligner allowing a maximum of 3 mismatches. For the TAD sequencing reads, cloning adapter sequences were first removed from both ends using cutadapt with CCAGTGTGGTGGAATTCTGCAGATATCAACAAGTTTGTACAAAAAAGTTGGCGGAAGTC AGGGTAGCGGAAGT (SEQ ID NO: 20) for 5′ and CCGCCACTGTGCTGGATATCAACCACTTTGTACAAGAAAGTTGGGTAGCCTTCGCGTTC AACACTACCTCC (SEQ ID NO: 21) for 3′ adapters. Bowtie reference was generated, and reads were mapped using Bowtie v1.2.3 allowing 0 mismatches. To identify activators, the edgeR package (Robinson et al., 2010) was used to calculate log 2 fold change, p-value, and false discovery rate (FDR) for each ORF by comparing changes in counts from sorted samples to unsorted cells.

Arrayed recruitment assays. Reporter cells stably co-expressing ABI-dCas9 and TetO gRNA were seeded either on 48-well or 96-well plates (Sarstedt) to reach 50-70% confluency on the day of transfection. 150 ng of each construct to be tested was transfected with polyethylenimine (PEI) at a ratio of 0.6 μl reagent. The day after transfection, recruitment was induced by treatment with ABA (100 μM). For tethered reporter assays in the presence of inhibitors, recruitment was similarly induced with 100 μM ABA the day after transfection but in the presence of either an inhibitor or the same volume of any additional DMSO. Inhibitors were dissolved in DMSO to a stock concentration of 10 mM. The final concentrations used were 100 nM for flavopiridol, 300 nM for JQ1, 1 μM for A-485, 2.5 μM for CX-4945 and 3 μM for AZ191. All inhibitors were a kind gift from the Structural Genomics Consortium (SGC). 48 hours after induction, cells were dissociated and resuspended in flow buffer using a liquid handing robot (TECAN) and analyzed by LSR Fortessa (BD). Cells were gated on high EBFP2 and RFP as a measure of gRNA and transfection control, respectively. Flow cytometry data was analyzed using FlowJo (v10).

SRF reporter assay. 30,000 NIH-3T3 cells on 24-well plates were transfected with 8 ng SRF reporter (p3DA.luc), 20 ng reference reporter (pcDNA3.1-Nanoluc-3×FLAG-V5) and 50 ng 3×FLAG tagged constructs (Addgene #87063). Transfection was carried out using Lipofectamine 3000 reagent (Thermo Fisher Scientific, L3000001) according to the manufacturer's protocols. Luciferase constructs were a kind gift from Dr. Maria Vartiainen (University of Helsinki). Cells were maintained in low-serum media (0.5% BCS) for 18 hours and stimulated for 7 hours (15% BCS), after which luciferase activity was measured. Firefly luciferase was normalized to Renilla luciferase activity using data from four independent transfections.

RNA sequencing and analysis. NIH-3T3 cells with stable integrations of SRF, C3orf62, SRF-C3orf62 or Nanoluc tagged at the C-terminus with EGFP were induced with 1 μg/mL doxycycline for 24 hours. RNA was extracted from cells maintained in low-serum conditions (0.5% calf serum) for 22 hours using RNeasy purification kit (Qiagen) and treated with DNase on column. Samples were induced and collected in technical duplicates from 6-well plates. Libraries were prepared using the NEBNext Ultra II Directional RNA-seq with Poly-A selection kit, pooled and sequenced on a 100-cycle NovaSeq 6000 SP. Reads were aligned to the Gencode mouse primary assembly (GRCm39) with STAR aligner v2.7.8a. Counts for each gene were generated using the Gencode vM26 transcript annotations. Changes in gene expression compared to cells expressing Nanoluc-EGFP were quantified using the edgeR package (Robinson et al., 2010).

Mass spectrometry samples. Entry clones were from the human ORFeome collection (Yang et al., 2011). Clones were transferred into pDEST-pcDNA5 vector carrying a C-terminal BiolD2-FLAG tag (Kim et al., 2016) using Gateway recombinase. Stable HEK293 FIp-In T-REx cell lines were generated as previously reported (Piette et al., 2021).

For AP-MS, cells were grown to 70% confluence on 150 mm dishes before inducing bait expression with 1 μg/mL tetracycline for 24 hours. Cells were then washed once with 1×PBS, scraped, pelleted, flash-frozen, and stored at −80° C. until processing. AP-MS was performed as previously described (Lambert et al., 2015). Briefly, cells were resuspended in cold lysis buffer (50 mM HEPES-NaOH pH 8.0, 100 mM KCl, 2 mM EDTA, 0.1% NP40, 10% glycerol, 1 mM PMSF, 1 mM DTT, 15 nM Calyculin A and protease inhibitor cocktail (Sigma-Aldrich P8340)) using a 1:4 pellet weight:volume ratio. Cells were lysed by one round of freeze-thaw, and lysates sonicated at 4° C. using three 10-second bursts at 35% amplitude with 2 s pauses. Sonicated lysate was treated with 100 U benzonase for 30 minutes at 4° C. prior to clearing by centrifugation at 20,000 g for 20 minutes at 4° C. An equal amount of supernatant from all samples processed within a batch was transferred to a tube containing 25 μL of pre-washed anti-FLAG magnetic bead 50% slurry (Sigma, M8823) and incubated for two hours at 4° C. Beads were recovered by magnetization and the supernatant discarded. Beads were washed once in lysis buffer, and once in 20 mM Tris-HCl pH8.0 with 2 mM CaCl₂ and digested on beads with trypsin in two stages (1 μg trypsin for 4 hours followed by the addition of 0.5 μg trypsin to the supernatant and overnight incubation at 37° C.), as previously described (Taipale et al., 2014). Finally, samples were acidified with 5% formic acid (final concentration) and stored at −80° C.

For BiolD, cells were grown to 70% confluence in 150 mm dishes before inducing gene expression with 1 μg/mL tetracycline for 18 hours. 50 μM biotin was then added to each plate for 6 hours. Cell pellets were collected as for AP-MS, and resuspended in lysis buffer (50 mM Tris-HCl pH 7.5, 150 mM NaCl, 0.1% SDS, 1% Igepal CA-630, 1 mM EDTA, 1 mM MgCl₂, protease inhibitor cocktail (Sigma-Aldrich P8340, 1:500), and 0.5% sodium deoxycholate) using a 1:10 pellet weight:volume ratio. After sonication, each sample was treated with 250 U Turbonuclease (BioVision 9207-50KU) and 1 μL RNase A solution (Sigma-Aldrich R6148) and incubated for 30 minutes at 4° C. SDS was then added to a final concentration of 0.25% and after mixing the samples were incubated for another 10 minutes at 4° C. followed by centrifugation at 20,000 g for 20 minutes. The supernatant was transferred to a tube containing 30 μl of pre-washed packed streptavidin beads (GE Healthcare, 17-5113-01). Streptavidin pulldown was done for 3 hours at 4° C. Beads were washed once in 1 ml of SDS buffer (2% SDS/50 mM Tris-HCl pH7.5), once in 1 ml lysis buffer, and once in TAP buffer (50 mM HEPES-KOH pH 8.0, 100 mM KCl, 10% glycerol, 2 mM EDTA, 0.1% Igepal CA-630), followed by three 1 ml washes with 50 mM ammonium bicarbonate pH 8.0. After the washes, beads were resuspended in ABC buffer containing 1 μg trypsin and incubated overnight at 37° C. The following day, the supernatant was collected and the streptavidin beads were washed with 50 μl water, which was combined with the first supernatant fraction. 0.5 μg trypsin was added to the combined supernatant sample, which was then incubated at 37° C. for 4 hours. Beads were then spun down and the supernatant recovered. Beads were rinsed twice using ABC buffer and these rinses were combined with the original supernatant. Combined supernatants were dried by centrifugal evaporation.

Mass spectrometry data acquisition and analysis. Samples were analyzed on a TripleTOF 5600 instrument (AB SCIEX, Concord, Ontario, Canada) as previously described (Piette et al., 2021) using Data-Dependent Acquisition (DDA). Data were processed and analyzed as previously described (Piette et al., 2021), using Proteowizard (Adusumilli and Mallick, 2017) implemented in ProHits v4.0 (Liu et al., 2016), Mascot, and Comet (Eng et al., 2013). The results were subsequently analyzed with the Trans-Proteomic Pipeline using iProphet (Shteynberg et al., 2011), and proteins with an iProphet probability 0.95 were further analyzed.

Significant interactors were identified with SAINTexpress (Teo et al., 2014). EGFP-BiolD2-FLAG and EGFP-BiolD2-FLAG were used as negative controls, using 2-fold compression for stringency as previously described (Mellacheruvu et al., 2013). SAINTexpress analysis used default parameters, and prey proteins were considered significant if they passed calculated Bayesian FDR cutoff of ≤5%. Dot plot figures were generated with ProHits-viz webserver (Knight et al., 2017).

### Example 8. Multiple TADs can be Combined to Activate Transcription

Multiple TADs were fused together with PYL1 and tested for transcriptional activity using the PYL1-ABI EGFP reporter system described above. The following TAD fragments were used: CITED1-8 (SEQ ID NO: 47), CITED2-12 (SEQ ID NO: 49), C3orf62-12 (SEQ ID NO: 45), BRD8-25 (SEQ ID NO: 40), ZXDC-12 (SEQ ID NO: 97), KLF7-1 (SEQ ID NO: 72), ATXN7L3-1 (SEQ ID NO: 35), FAM90A1-20 (SEQ ID NO: 60), SPDYE4-3 (SEQ ID NO: 90), YAF2-7 (SEQ ID NO: 96).

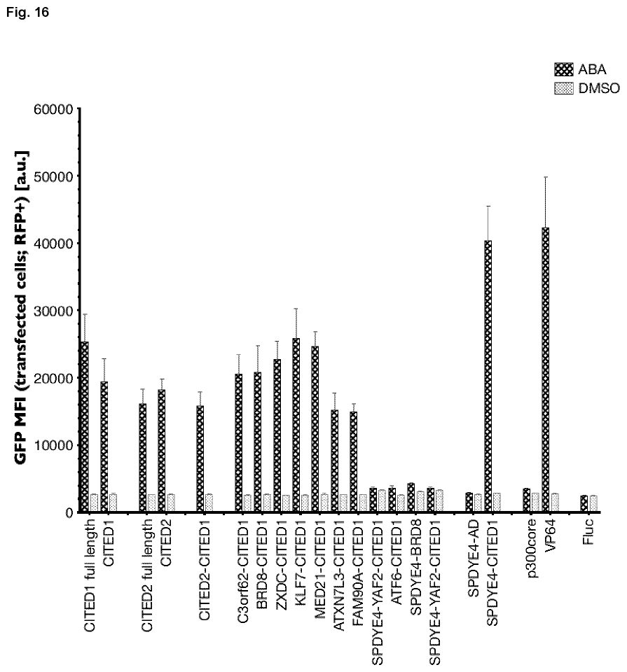

As shown in FIG. 16, the SPDYE4-CITED1 TAD fusion protein resulted in a marked increase in transcriptional activation compared to either TAD alone.

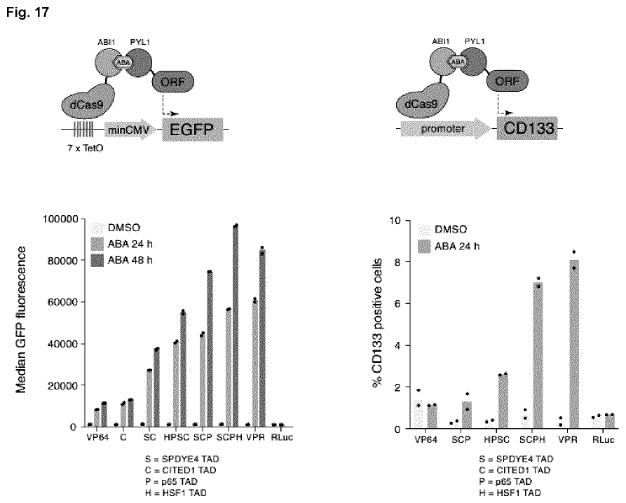

Up to 2 additional TADs selected from p65 and HSF1 were combined with SPDYE4-CITED1 TADs in different orders and tested for the ability to activate transcription of the PYL1-ABI EGFP reporter system (FIG. 17, left panel) or the endogenous CD133 gene (FIG. 17, right panel). The SPDYE4-CITED1-P65-HSF1 (SCPH) effector provided the most robust activation in both systems tested.

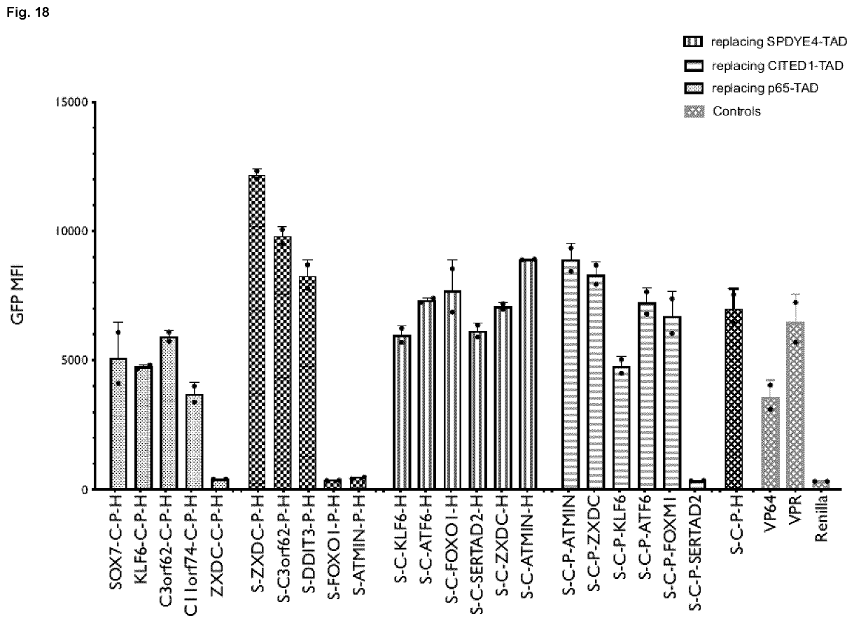

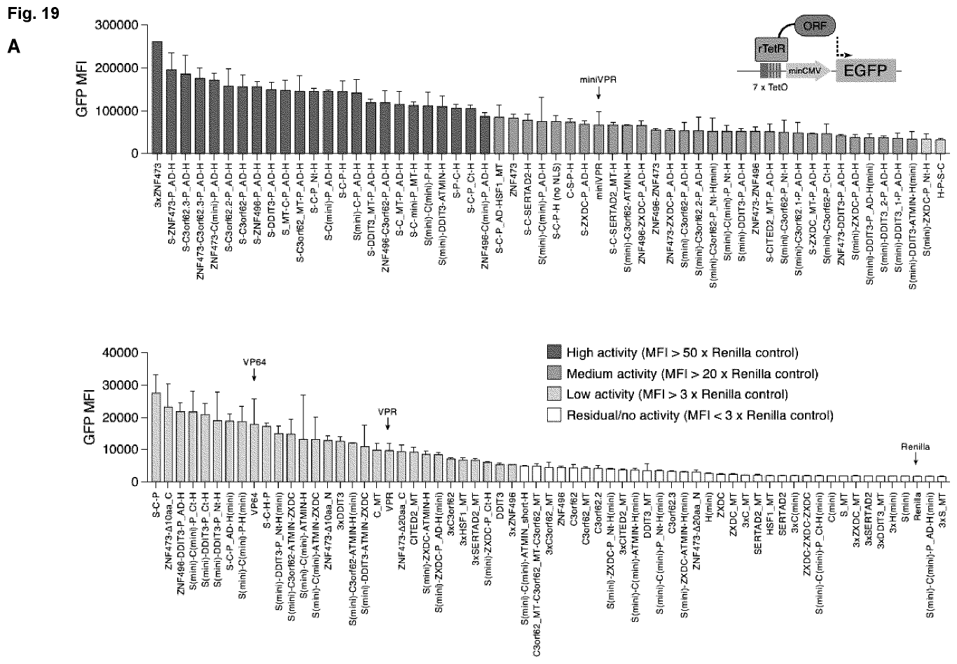

To test additional TAD combinations, each part of the multi-component SPDYE4-CITED1-P65-HSF1 (SCPH) effector was individually replaced with another TAD identified in Example 4 (see e.g. Table 1). Results are shown in FIG. 18.

To test whether shorter TAD sequences could be used in the SCPH effector, each TAD was independently replaced with a short or mini TAD component. Results are shown in FIG. 15. Mini-TAD sequences were selected as follows:

miniSPDYE4 (SEQ ID NO: 102): Sequence was selected based on the overlap between the two enriched fragments from our tiling screen of the SPDYE4 full length protein. The minimal sequence was designed to include either the acidic rich region or the following beta hairpin.

miniHSF1: The 150 amino acid C-terminal region of HSF1 which contains its activation domain comprises of two known TADs. The longer TAD is between amino acids 431-529 and the shorter one (“mini-HSF1 (401-420)” or “H(mini)”; SEQ ID NO: 119) is between amino acids 401-420. We selected the shorter domain rich in hydrophobic residues to test due to its more compact size and as it was previously reported to have more potency than the longer TAD. (Newton et al., 1996; 10.1128/MCB.16.3.839)

miniP65: RelA or p65 is known to have two distinct transactivation domains within its C-terminus. The first TAD (“P(mini N-term)”; SEQ ID NO: 105) comprises amino acids 428-520 and the second (“P(mini C-term)”; SEQ ID NO: 106) is the proceeding 521-551 residues. (Schmitz and Baeuerle, 1991; 10.1002/j.1460-2075.1991.tb04950.x)

miniCITED1 (“C(mini)”; SEQ ID NO: 103) was selected to include the C-terminal acidic-rich region within the overlapping region between CITED1-7 and CITED1-8.

Methods: Cells were seeded on 48-well plates. Next day, after cells were between 70-90% confluent, 250 ng of each construct was transfected in each well using (Thermo Fisher Scientific, 11668019) reagent. Each construct was either directly fused TagRFP either directly fused to each component or being co-expressed from the same plasmid was used as a measure of transfection. In each experiment, the same gate for RFP+ cells were used to control for the effect of each construct's expression levels on activity. EGFP reporter cells were expanded from a clonal line to control for level of ABI-dCas9 and gRNA targeting the TetO7 sites upstream of the promoter. For the CD133 induction experiment, a 293T cell line stably expressing ABI-dCas9 and a pool of 5 gRNAs targeting the promoter were used for all activators. CD133 antibody conjugated to APC (Miltenyi Biotec, 130-113-668) was used. All activator constructs were fused to PYL1 at their C-terminus and were recruited to their targets by treating the cells with 1 μM abscisic acid for either 24 or 48 hours.

### Example 9

A further 117 different combinations of activation domains or fragments of individual activation domains were assayed by fusing them either to PYL1 (to test with the dCas9-ABI1 system) or to rTetR, a transcription factor that binds DNA in the presence of doxycycline. All constructs were tested with the same TetO reporter construct. Large differences were observed in the activity of these constructs, ranging from no activity to very high potency (FIG. 19A-B). In general, there was a good correlation between the results with rTetR and dCas9 based recruitment systems (FIG. 19C), suggesting that most activation domains function similarly in both contexts. However, for example VPR was much less active as an rTetR fusion than as a dCas9 fusion. Consistent with previous experiments, SCPH and its variants were still the most active constructs in the system. For example, a construct containing a miniaturized activation domain of C3orf62 instead of that from CITED1 (“C” in the SCPH construct) and a shorter version of p65 activation domain (“P”) was more potent than the original SCPH construct despite being significantly smaller (1059 bp vs 1611 bp) (FIG. 19A-B).

Methods: 200 ng of plasmid expressing super activators fused to rTetR in their C terminus and 50 ng DsRed was transfected into HEK293T cells stably expressing 7×TetO-EGFP reporter. One day after transfection, cells were treated with 1 μg/ml doxycycline for 48 hours. After the treatment, cells were analyzed by flow cytometry for EGFP expression

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims. The scope of the claims should not be limited by the preferred embodiments and examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety.

TABLE of Sequences

Glycine serine linker (SEQ ID NO: 6) SGGSGGS
Glycine serine linker (SEQ ID NO: 7) SGGS
Glycine serine linker (SEQ ID NO: 8) GSGSGS
linker (SEQ ID NO: 9) INSRSSGS
NLS (SEQ ID NO: 22) PKKKRKV
Linker + NLS (SEQ ID NO: 141) INSRSSGSPKKKRKVGS
>YAF2_RYBP domain of YAF2 (SEQ ID NO: 147)
RPRLKNVDRSSAQHLEVTVGDLTVIITDFKEKT
>YAF2_RYBP domain of RYBP (SEQ DI NO: 148)
RPRLKNVDRSTAQQLAVTVGNVTVIITDFKEKT
>CBX-C domain of CBX2 (SEQ ID NO: 149)
QDWKPTRSLIEHVFVTDVTANLITVTVKESPTSV
>CBX-C domain of CBX6 (SEQ ID NO: 150)
GDWRPEMSPCSNVVVTDVTSNLLTVTIKEFCNPE
>CBX-C domain of CBX8 (SEQ ID NO: 151)
ESWSPSLTNLEKVVVTDVTSNFLTVTIKESNTDQ
>CBX-C domain of CBX4 (SEQ ID NO: 152)
SEFKPFFGNIIITDVTANCLTVTFKEYVTV
>CBX-C domain of CBX7 (SEQ ID NO: 153)
PPWTPALPSSEVTVTDITANSITVTFREAQAAE
>SPDYE4-2 (SEQ ID NO: 154)
VRSPEVVVDDEVPGPSAPWIDPSPQPQSLGLKRKSEWSDESEEELEEEL
ELERAPEPEDT
>SPDYE4-5 (SEQ ID NO: 155)
WWVETLCGLKMKLKRKRASSVLPEHHEAFNRLLGDPVVQKFLAWDKDLR
VSDKYLLAMVI

TABLE 1
Fragments identified as TADs by TADseq. Fragments marked with * were independently validated.

| | | | SEQ | logFC | FDR | Hit | logFC | FDR | Hit |
|---|---|---|---|---|---|---|---|---|---|
| | | | ID | high | high | high | medium | medium | medium |
| Gene | Fragment | Sequence | NO: | GFP | GFP | GFP | GFP | GFP | GFP |
| ATF6 | ATF6-1 | MGEPAGVAGTMESPFSPGLFHRLDEDWDS | 29 | 3.145276698 | 0.02121147 | hit | 1.563296241 | 6.04E−06 | hit |
| | | ALFAELGYFTDTDELQLEAANETYENNFDNL | | | | | | | |
| ATF6 | ATF6-2* | HRLDEDWDSALFAELGYFTDTDELQLEAAN | 30 | 4.68756189 | 2.94E−05 | hit | 1.477263549 | 4.41E−05 | hit |
| | | ETYENNFDNLDFDLDLVPWESDIWDINNQI | | | | | | | |
| ATF6B | ATF6B-1 | MAELMLLSEIADPTRFFTDNLLSPEDWGLQ | 31 | 1.916896584 | 0.00665441 | hit | 0.993094738 | 0.001024406 | hit |
| | | NSTLYSGLDEVAEEQTQLFRCPEQDVPFDG | | | | | | | |
| ATMIN | ATMIN-24 | NPGPDTQLPSGPAQNPGIDFDIEEFFSASNI | 32 | 1.232649648 | 0.05344855 | | 0.98800755 | 0.000965273 | hit |
| | | QTQTEESELSTMTTEPVLESLDIETQTDF | | | | | | | |
| ATMIN | ATMIN-32 | ETQTMSSGFETLGSLFFTSNETQTAMDDFL | 33 | 0.63551575 | 0.3055137 | | 0.819759214 | 0.009050433 | hit |
| | | LADLAWNTMESQFSSVETQTSAEPHTVSNF | | | | | | | |
| ATOH1 | ATOH1-1 | MSRLLHAEEWAEVKELGDHHRQPQPHHLP | 34 | 0.721822453 | 0.30506874 | | 0.919351522 | 0.017130359 | hit |
| | | QPPPPPQPPATLQAREHPVYPPELSLLDST | | | | | | | |
| | | D | | | | | | | |
| ATXN7L3 | ATXN7L3- | MKMEEMSLSGLDNSKLEAIAQEIYADLVEDS | 35 | 1.637901718 | 0.02573555 | hit | 1.683893477 | 1.80E−05 | hit |
| | 1* | CLGFCFEVHRAVKCGYFFLDDTDPDSMKD | | | | | | | |
| ATXN7L3 | ATXN7L3- | QEIYADLVEDSCLGFCFEVHRAVKCGYFFLD | 36 | 0.765431613 | 0.28306469 | | 0.891059001 | 0.002440708 | hit |
| | 2 | DTDPDSMKDFEIVDQPGLDIFGQVFNQWK | | | | | | | |
| ATXN7L3 | ATXN7L3- | FEIVDQPGLDIFGQVFNQWKSKECVCPNCS | 37 | 0.764101911 | 0.20450075 | | 0.751439597 | 0.022410022 | hit |
| | 4 | RSIAASRFAPHLEKCLGMGRNSSRIANRRI | | | | | | | |
| BRD8 | BRD8-23 | KEECFRSGVAEAPVGSKAPSIDGKEELDLA | 38 | 0.588420958 | 0.35787782 | | 0.591494577 | 0.032988887 | hit |
| | | EKMDIAVSYTGEELDFETVGDIIAIIEDKV | | | | | | | |
| BRD8 | BRD8-24 | IDGKEELDLAEKMDIAVSYTGEELDFETVGDI | 39 | 1.593193215 | 0.04662626 | hit | 1.565645971 | 2.69E−05 | hit |
| | | IAIIEDKVDDHPEVLDVAAVEAALSFCE | | | | | | | |
| BRD8 | BRD8-25* | GEELDFETVGDIIAIIEDKVDDHPEVLDVAAV | 40 | 1.567478812 | 0.02072139 | hit | 1.833747802 | 6.04E−06 | hit |
| | | EAALSFCEENDDPQSLPGPWEHPIQQER | | | | | | | |
| BTBD18 | BTBD18- | IDCREPYAFDTALLEQPCEAEEYRITSAAAT | 41 | 1.246176755 | 0.15033388 | | 0.856107889 | 0.003217538 | hit |
| | 24 | SELEEILDFMLCGSDIEPPIGSLESPGAE | | | | | | | |
| BTBD18 | BTBD18- | EEYRITSAAATSELEEILDFMLCGSDIEPPIG | 42 | 1.470635679 | 0.07002876 | | 1.413328465 | 7.30E−05 | hit |
| | 25 | SLESPGAEGCRTPTYHLTETGKNWIEGE | | | | | | | |
| C11orf74 | C11orf74- | MSAHMSGLEIMDEDQLIKDVLDKFLNCHEQ | 43 | 2.356356482 | 0.012114 | hit | 1.551234476 | 7.99E−06 | hit |
| | 1 | TYDEEFLNTFTHLSQDLLLLPGEVEQDVST | | | | | | | |
| C3orf62 | C3orf62- | NHMCGHCQDSPFKEEAWALLMDKSPQKAT | 44 | 6.02271451 | 7.93E−06 | hit | −0.526078624 | 0.221475633 | |
| | 11 | DADPGSLKQAFDDHNIVETVLDLEEDYNVM | | | | | | | |
| | | T | | | | | | | |
| C3orf62 | C3orf62- | QDSPFKEEAWALLMDKSPQKATDADPGSL | 45 | 6.492950845 | 6.25E−06 | hit | −0.606664208 | 0.124701907 | |
| | 12* | KQAFDDHNIVETVLDLEEDYNVMTSFKYQIE | | | | | | | |
| CITED1 | CITED1-7 | LQNWDFGAQAGGAESLSPSAGAQSPAIIDS | 46 | 4.31381114 | 8.10E−05 | hit | 1.596638925 | 0.000505651 | hit |
| | | DPVDEEVLMSLVVELGLDRANELPELWLGQ | | | | | | | |
| CITED1 | CITED1- | ESLSPSAGAQSPAIIDSDPVDEEVLMSLVVE | 47 | 6.826260035 | 6.25E−06 | hit | −1.165531748 | 0.000242099 | |
| | 8* | LGLDRANELPELWLGQNEFDFTADFPSSC | | | | | | | |
| CITED2 | CITED2- | MPASVAHVPAAMLPPNVIDTDFIDEEVLMSL | 48 | 6.164977472 | 6.48E−06 | hit | 0.949095547 | 0.000563063 | |
| | 11 | VIEMGLDRIKELPELWLGQNEFDFMTDFV | | | | | | | |
| CITED2 | CITED2- | AMLPPNVIDTDFIDEEVLMSLVIEMGLDRIKE | 49 | 6.147579295 | 7.93E−06 | hit | −0.900478069 | 0.002440708 | |
| | 12* | LPELWLGQNEFDFMTDFVCKQQPSRVSC | | | | | | | |
| CSRNP1 | CSRNP1- | DNIEAPHFPLPGLSPPGDASSCFLESLMGFS | 50 | 0.502869407 | 0.46018996 | | 0.695535008 | 0.015779736 | hit |
| | 28 | EPAAEALDPFIDSQFEDTVPASLMEPVPV | | | | | | | |
| DDIT3 | DDIT3-1 | MAAESLPFSFGTLSSWELEAWYEDLQEVLS | 51 | 2.372684704 | 0.00646538 | hit | 1.501778634 | 6.04E−06 | hit |
| | | SDENGGTYVSPPGNEEEESKIFTTLDPASL | | | | | | | |
| DDIT3 | DDIT3-2 | WYEDLQEVLSSDENGGTYVSPPGNEEEES | 52 | 1.138253747 | 0.11502125 | | 1.279979309 | 0.000330392 | hit |
| | | KIFTTLDPASLAWLTEEEPEPAEVTSTSQSP | | | | | | | |
| ELF4 | ELF4-1 | MAITLQPSDLIFEFASNGMDDDIHQLEDPSV | 53 | 0.662555474 | 0.25787399 | | 0.728708968 | 0.015524531 | hit |
| | | FPAVIVEQVPYPDLLHLYSGLELDDVHNG | | | | | | | |
| ELF4 | ELF4-2 | DDIHQLEDPSVFPAVIVEQVPYPDLLHLYSG | 54 | 1.466757956 | 0.02072139 | hit | 1.363697693 | 6.52E−05 | hit |
| | | LELDDVHNGIITDGTLCMTQDQILEGSFL | | | | | | | |
| FAM22F | FAM22F- | PRPQRPAETNAHLPPPRPQRPAETKVPEEI | 55 | 0.999831925 | 0.17807384 | | 0.988353867 | 0.015779736 | hit |
| | 19 | PPEVVQEYVDIMEELLGSHPGDTGEPEGQR | | | | | | | |

| | | | | | | | | | |
|---|---|---|---|---|---|---|---|---|---|
| FAM22F | FAM22F- | PAETKVPEEIPPEVVQEYVDIMEELLGSHPG | 56 | 3.214335785 | 0.00094772 | hit | 2.101012353 | 6.76E−07 | hit |
| | 20 | DTGEPEGQREKGKVEQPQEEDGITSDPGL | | | | | | | |
| FAM22F | FAM22F- | EKGKVEQPQEEDGITSDPGLLSYIDKLCSQE | 57 | 4.760133648 | 8.76E−06 | hit | 2.324879314 | 1.46E−05 | hit |
| | 22 | DFVTKVEAVIHPRFLEELLSPDPQMDFLA | | | | | | | |
| FAM22F | FAM22F- | LSYIDKLCSQEDFVTKVEAVIHPRFLEELLSP | 58 | 5.822230419 | 6.25E−06 | hit | 0.273685646 | 0.466208757 | |
| | 23 | DPQMDFLALSQELEQEEGLTLAQLVEKR | | | | | | | |
| FAM90A1 | FAM90A1- | RQPPHSRPCLPTAQACTMSHHPAASHDGA | 59 | 2.051462115 | 0.03110723 | hit | 1.556796738 | 7.06E−05 | hit |
| | 19 | QPLRVLFRRLENGRWSSSLLTAPSFHSPEK | | | | | | | |
| | | P | | | | | | | |
| FAM90A1 | FAM90A1- | HPAASHDGAQPLRVLFRRLENGRWSSSLLT | 60 | 3.016551155 | 0.00281867 | hit | 1.616164006 | 0.0002923 | hit |
| | 20* | APSFHSPEKPGAFLAQSPHVSEKSEGPCVR | | | | | | | |
| HOXA2 | HOXA2-4 | PGSHPRHGAGGRPKPSPAGSRGSPVPAGA | 61 | 1.664427181 | 0.23442147 | | 1.594816728 | 0.021805285 | hit |
| | | LQPPEYPWMKEKKAAKKTALLPAAAAAATA | | | | | | | |
| | | A | | | | | | | |
| HOXA2 | HOXA2-5 | RGSPVPAGALQPPEYPWMKEKKAAKKTALL | 62 | 1.684064934 | 0.24906108 | | 2.31148338 | 0.000384794 | hit |
| | | PAAAAAATAAATGPACLSHKESLEIADGSG | | | | | | | |
| HOXA2 | HOXA2-6 | KKAAKKTALLPAAAAAATAAATGPACLSHKE | 63 | 1.951001869 | 0.24906108 | | 1.737795426 | 0.015779736 | hit |
| | | SLEIADGSGGGSRRLRTAYTNTQLLELEK | | | | | | | |
| HSF1 | HSF1-20 | KNELSDHLDAMDSNLDNLQTMLSSHGFSVD | 64 | 0.623357487 | 0.30974031 | | 0.707735867 | 0.026141316 | hit |
| | | TSALLDLFSPSVTVPDMSLPDLDSSLASIQ | | | | | | | |
| HSF1 | HSF1-21 | MLSSHGFSVDTSALLDLFSPSVTVPDMSLP | 65 | 0.431982684 | 0.45359875 | | 0.899190622 | 0.002496242 | hit |
| | | DLDSSLASIQELLSPQEPPRPPEAENSSPD | | | | | | | |
| HSF1 | HSF1-24 | SGKQLVHYTAQPLFLLDPGSVDTGSNDLPV | 66 | 0.676280443 | 0.54433041 | | 0.809218277 | 0.008149744 | hit |
| | | LFELGEGSYFSEGDGFAEDPTISLLTGSEP | | | | | | | |
| HSF1 | HSF1-25 | AQPLFLLDPGSVDTGSNDLPVLFELGEGSY | 67 | 0.772548781 | 0.22014105 | | 1.152582348 | 0.0002923 | hit |
| | | FSEGDGFAEDPTISLLTGSEPPKAKDPTVS | | | | | | | |
| JAZF1 | JAZF1-1 | MTGIAAASFFSNTCRFGGCGLHFPTLADLIE | 68 | 0.284758647 | 0.6730689 | | 0.663877674 | 0.031991955 | hit |
| | | HIEDNHIDTDPRVLEKQELQQPTYVALSY | | | | | | | |
| KLF15 | KLF15-6 | GPVAWGPWRRAAAPVKGEHFCLPEFPLGD | 69 | 2.466557755 | 0.00312371 | hit | 1.20185423 | 0.000203701 | hit |
| | | PDDVPRPFQPTLEEIEEFLEENMEPGVKEVP | | | | | | | |
| KLF6 | KLF6-1 | MDVLPMCSIFQELQIVHETGYFSALPSLEEY | 70 | 5.453107649 | 6.25E−06 | hit | 1.867753689 | 6.47E−06 | hit |
| | | WQQTCLELERYLQSEPCYVSASEIKFDSQ | | | | | | | |
| KLF7 | KLF7-1* | MDVLASYSIFQELQLVHDTGYFSALPSLEET | 72 | 5.997661437 | 6.25E−06 | hit | 1.289603101 | 0.002206825 | hit |
| | | WQQTCLELERYLQTEPRRISETFGEDLDC | | | | | | | |
| KLF7 | KLF7-2 | YFSALPSLEETWQQTCLELERYLQTEPRRIS | 73 | 1.430957654 | 0.17621033 | | 1.165159905 | 0.000666098 | hit |
| | | ETFGEDLDCFLHASPPPCIEESFRRLDPL | | | | | | | |
| LIN9 | LIN9-25 | SRLTAILLQIKCLAEGGDLNSFEFKSLTDSLN | 74 | 2.093964257 | 0.03526736 | hit | 1.07996726 | 0.075362785 | |
| | | DIKSTIDASNISCFQNNVEIHVAHIQSG | | | | | | | |
| MYCBP | MYCBP-1 | MAHYKAADSKREQFRRYLEKSGVLDTLTKV | 75 | 1.01658798 | 0.11503602 | | 0.729980644 | 0.007112247 | hit |
| | | LVALYEEPEKPNSALDFLKHHLGAATPENP | | | | | | | |
| MYCL1 | MYCL1-1 | MDYDSYQHYFYDYDCGEDFYRSTAPSEDI | 76 | 1.136770705 | 0.05636799 | | 0.796782271 | 0.01156406 | hit |
| | | WKKFELVPSPPTSPPWGLGPGAGDPAPGI | | | | | | | |
| | | GP | | | | | | | |
| MYOD1 | MYOD1-2 | CSFATTDDFYDDPCFDSPDLRFFEDLDPRL | 77 | 0.702539998 | 0.30142347 | | 0.848602054 | 0.002496242 | hit |
| | | MHVGALLKPEEHSHFPAAVHPAPGAREDEH | | | | | | | |
| NEUROD1 | NEUROD1- | LRRMKANARERNRMHGLNAALDNLRKVVP | 78 | 0.670794426 | 0.29541863 | | 0.953338619 | 0.001354227 | hit |
| | 6 | CYSKTQKLSKIETLRLAKNYIWALSEILRSG | | | | | | | |
| NEUROG3 | NEUROG3- | RRSRRKKANDRERNRMHNLNSALDALRGV | 79 | 0.060387059 | 0.91793719 | | 0.671331782 | 0.038396908 | hit |
| | 5 | LPTFPDDAKLTKIETLRFAHNYIWALTQTLR | | | | | | | |
| NPAS4 | NPAS4-31 | DVPLVPEGLLTPEASPVKQSFFHYSEKEQN | 80 | 1.220084575 | 0.14208522 | | 0.85633903 | 0.034713147 | hit |
| | | EIDRLIQQISQLAQGMDRPFSAEAGTGGLE | | | | | | | |
| NPAS4 | NPAS4-34 | PLGGLEPLDSNLSLSGAGPPVLSLDLKPWK | 81 | 0.945976876 | 0.14289484 | | 0.918642828 | 0.003190486 | hit |
| | | CQELDFLADPDNMFLEETPVEDIFMDLSTP | | | | | | | |
| NPAS4 | NPAS4-36 | DNMFLEETPVEDIFMDLSTPDPSEEWGSGD | 82 | 0.670986209 | 0.2556082 | | 1.209379094 | 6.61E−05 | hit |
| | | PEAEGPGGAPSPCNNLSPEDHSFLEDLATY | | | | | | | |
| NPAS4 | NPAS4-37 | DPSEEWGSGDPEAEGPGGAPSPCNNLSPE | 83 | 1.645174283 | 0.01967705 | hit | 0.701878979 | 0.055839582 | |
| | | DHSFLEDLATYETAFETGVSAFPYDGFTDEL | | | | | | | |
| NPAS4 | NPAS4-39 | CNNLSPEDHSFLEDLATYETAFETGVSAFPY | 84 | 0.798049889 | 0.3822513 | | 0.768390862 | 0.019277289 | hit |
| | | DGFTDELHQLQSQVQDSFHEDGSGGEPTF | | | | | | | |
| RBPJ | RBPJ-13 | GMALPRLIIRKVDKQTALLDADDPVSQLHKC | 85 | 1.431035271 | 0.02573555 | hit | 0.031481589 | 0.974213707 | |
| | | AFYLKDTERMYLCLSQERIIQFQATPCPK | | | | | | | |
| RELB | RELB-2 | RRVARPPAAPELGALGSPDLSSLSLAVSRS | 86 | 2.146540297 | 0.00281867 | hit | 1.710867835 | 6.99E−06 | hit |
| | | TDELEIIDEYIKENGFGLDGGQPGPGEGLP | | | | | | | |
| RELB | RELB-3 | SSLSLAVSRSTDELEIIDEYIKENGFGLDGGQ | 87 | 1.768392783 | 0.01126942 | hit | 1.802901174 | 6.76E−07 | hit |
| | | PGPGEGLPRLVSRGAASLSTVTLGPVAP | | | | | | | |
| SERTAD1 | SERTAD1- | IDTSMYDNELWAPASEGLKPGPEDGPGKEE | 88 | 0.234946196 | 0.84161029 | | 0.838507882 | 0.032988887 | hit |
| | 10 | APELDEAELDYLMDVLVGTQALERPPGPGR | | | | | | | |
| SERTAD2 | SERTAD2- | SEAGTQKLDGPQESRADDSKLMDSLPGNF | 89 | 2.480489855 | 0.00665441 | hit | 1.821766531 | 4.96E−06 | hit |
| | 11 | EITTSTGFLTDLTLDDILFADIDTSMYDFDP | | | | | | | |
| SPDYE4 | SPDYE4- | DPSPQPQSLGLKRKSEWSDESEEELEEELE | 90 | 4.820495878 | 2.26E−05 | hit | 2.40936057 | 1.30E−07 | hit |
| | 3* | LERAPEPEDTWVVETLCGLKMKLKRKRASS | | | | | | | |
| SPDYE4 | SPDYE4- | SEEELEEELELERAPEPEDTWVVETLCGLK | 91 | 4.316060433 | 0.00020711 | hit | 1.914291641 | 6.04E−06 | hit |
| | 4 | MKLKRKRASSVLPEHHEAFNRLLGDPVVQK | | | | | | | |
| SS18L2 | SS18L2-2 | QETIQRLLEENDQLIRCIVEYQNKGRGNECV | 92 | 1.01153592 | 0.09853126 | | 1.166148268 | 0.000386437 | hit |
| | | QYQHVLHRNLIYLATIADASPTSTSKAME | | | | | | | |
| TP53- | TP53- | PQSDPSVEPPLSQETFSDLWKLLPENNVLS | 93 | 1.108562999 | 0.28306469 | | 0.985578432 | 0.017581223 | hit |
| AD1-2 | AD1-2 | PLPSQAMDDLMLSPDDIEQWFTEDPGPDEA | | | | | | | |
| VP64 | VP64 | DALDDFDLDMLGSDALDDFDLDMLGSDALD | 94 | 5.47585036 | 6.48E−06 | hit | 2.374266586 | 1.76E−07 | |
| | | DFDLDMLGSDALDDFDLDMLGGSVEREG | | | | | | | |
| YAF2 | YAF2-6 | EKKDKVEKEKSEKETTSKKNSHKKTRPRLK | 95 | 4.315999866 | 0.00046506 | hit | 1.771487421 | 0.000612967 | hit |
| | | NVDRSSAQHLEVTVGDLTVIITDFKEKTKS | | | | | | | |
| YAF2 | YAF2-7* | SHKKTRPRLKNVDRSSAQHLEVTVGDLTVII | 96 | 4.986869359 | 1.91E−05 | hit | 1.828375134 | 7.30E−05 | hit |
| | | TDFKEKTKSPPASSAASADQHSQSGSSSD | | | | | | | |
| ZXDC | ZXDC-12* | SPAEQHGAQDTELSAGTGNFYLESGGSAR | 97 | 3.965748255 | 0.01967705 | hit | 2.496061078 | 8.08E−08 | hit |
| | | TDYRAIQLAKEKKQRGAGSNAGASQSTQRK | | | | | | | |
| ZXDC | ZXDC-13 | YLESGGSARTDYRAIQLAKEKKQRGAGSNA | 98 | 2.377281872 | 0.00307646 | hit | 1.734231119 | 8.57E−06 | hit |
| | | GASQSTQRKIKEGKMSPPHFHASQNSWLC | | | | | | | |
| | | G | | | | | | | |
| ZXDC | ZXDC-16 | SLVVPSGGRPGPAPAAGVQCGAQGVQVQL | 99 | 0.478496057 | 0.41728761 | | 0.729256459 | 0.017925843 | hit |
| | | VQDDPSGEGVLPSARGPATFLPFLTVDLPV | | | | | | | |
| | | Y | | | | | | | |

TABLE 2
Subset of fragments identified as TADs by TADseq from Table 1.

| | | | SEQ ID |
|---|---|---|---|
| Gene | Fragment | Sequence | NO: |
| ATMIN | ATMIN-24 | NPGPDTQLPSGPAQNPGIDFDIEEFFSASNIQTQTEESELS | 32 |
| | | TMTTEPVLESLDIETQTDF | |
| ATMIN | ATMIN-32 | ETQTMSSGFETLGSLFFTSNETQTAMDDFLLADLAWNTM | 33 |
| | | ESQFSSVETQTSAEPHTVSNF | |
| ATOH1 | ATOH1-1 | MSRLLHAEEWAEVKELGDHHRQPQPHHLPQPPPPPQPP | 34 |
| | | ATLQAREHPVYPPELSLLDSTD | |
| ATXN7L3 | ATXN7L3-1 | MKMEEMSLSGLDNSKLEAIAQEIYADLVEDSCLGFCFEVH | 35 |
| | | RAVKCGYFFLDDTDPDSMKD | |
| ATXN7L3 | ATXN7L3-2 | QEIYADLVEDSCLGFCFEVHRAVKCGYFFLDDTDPDSMK | 36 |
| | | DFEIVDQPGLDIFGQVFNQWK | |
| ATXN7L3 | ATXN7L3-4 | FEIVDQPGLDIFGQVFNQWKSKECVCPNCSRSIAASRFAP | 37 |
| | | HLEKCLGMGRNSSRIANRRI | |
| BRD8 | BRD8-23 | KEECFRSGVAEAPVGSKAPSIDGKEELDLAEKMDIAVSYT | 38 |
| | | GEELDFETVGDIIAIIEDKV | |
| BRD8 | BRD8-24 | IDGKEELDLAEKMDIAVSYTGEELDFETVGDIIAIIEDKVDD | 39 |
| | | HPEVLDVAAVEAALSFCE | |
| BRD8 | BRD8-25 | GEELDFETVGDIIAIIEDKVDDHPEVLDVAAVEAALSFCEEN | 40 |
| | | DDPQSLPGPWEHPIQQER | |
| BTBD18 | BTBD18-24 | IDCREPYAFDTALLEQPCEAEEYRITSAAATSELEEILDFML | 41 |
| | | CGSDIEPPIGSLESPGAE | |
| BTBD18 | BTBD18-25 | EEYRITSAAATSELEEILDFMLCGSDIEPPIGSLESPGAEGC | 42 |
| | | RTPTYHLTETGKNWIEGE | |
| CSRNP1 | CSRNP1-28 | DNIEAPHFPLPGLSPPGDASSCFLESLMGFSEPAAEALDP | 50 |
| | | FIDSQFEDTVPASLMEPVPV | |
| HOXA2 | HOXA2-4 | PGSHPRHGAGGRPKPSPAGSRGSPVPAGALQPPEYPW | 61 |
| | | MKEKKAAKKTALLPAAAAAATAA | |
| HOXA2 | HOXA2-5 | RGSPVPAGALQPPEYPWMKEKKAAKKTALLPAAAAAATA | 62 |
| | | AATGPACLSHKESLEIADGSG | |
| HOXA2 | HOXA2-6 | KKAAKKTALLPAAAAAATAAATGPACLSHKESLEIADGSG | 63 |
| | | GGSRRLRTAYTNTQLLELEK | |
| JAZF1 | JAZF1-1 | MTGIAAASFFSNTCRFGGCGLHFPTLADLIEHIEDNHIDTD | 68 |
| | | PRVLEKQELQQPTYVALSY | |
| LIN9 | LIN9-25 | SRLTAILLQIKCLAEGGDLNSFEFKSLTDSLNDIKSTIDASNI | 74 |
| | | SCFQNNVEIHVAHIQSG | |
| MYCBP | MYCBP-1 | MAHYKAADSKREQFRRYLEKSGVLDTLTKVLVALYEEPEK | 75 |
| | | PNSALDFLKHHLGAATPENP | |
| MYCL1 | MYCL1-1 | MDYDSYQHYFYDYDCGEDFYRSTAPSEDIWKKFELVPSP | 76 |
| | | PTSPPWGLGPGAGDPAPGIGP | |
| NEUROD1 | NEUROD1- | LRRMKANARERNRMHGLNAALDNLRKVVPCYSKTQKLSK | 78 |
| | 6 | IETLRLAKNYIWALSEILRSG | |
| NEUROG3 | NEUROG3- | RRSRRKKANDRERNRMHNLNSALDALRGVLPTFPDDAKL | 79 |
| | 5 | TKIETLRFAHNYIWALTQTLR | |
| RBPJ | RBPJ-13 | GMALPRLIIRKVDKQTALLDADDPVSQLHKCAFYLKDTER | 85 |
| | | MYLCLSQERIIQFQATPCPK | |

TABLE 3
Exemplary sequences of TADs and functional variants and active fragments thereof.

| | SEQ | |
|---|---|---|
| TAD | ID NO: | Sequence |
| YAF2 short | 100 | SHKKTRPRLKNVDRSSAQHLEVTVGDLTVIITDFKEKTKSPPASSA |
| C3orf62 short | 101 | MDKSPQKATDADPGSLKQAFDDHNIVETVLDLEEDYNVMTSFKYQ |
| | | IE |
| “miniSPDYE4” or | 102 | SDESEEELEEELELERAPEPEDTWVVETL |
| “S(mini)” | | |
| “miniCITED1” or | 103 | DSDPVDEEVLMSLVVELGLDRANEL |
| “C(mini)” | | |
| HSF1 short | 104 | GFSVDTSALLDLFSP |
| “p65 short (N- | 105 | PTQAGEGTLSEALLQLQFDDEDLGALLGNSTDPAVFTDLASVDNS |
| term)”; “P(mini N- | | EFQQ |
| term)”; or “P_Nt” | | |
| “p65 short (C- | 106 | APGLPNGLLSGDEDFSSIADMDFSALL |
| term)”; “P(mini C- | | |
| term)”; or “P_Ct” | | |
| ZXDC short | 107 | AGTGNFYLESGGSARTDYRAIQLAKEKKQRGAGSNAGASQSTQR |
| | | KIKEGKM |
| RELB short | 108 | SSLSLAVSRSTDELEIIDEYIKENGFGLDGGQPGPGEGLP |
| FAM22F short | 109 | LSYIDKLCSQEDFVTKVEAVIHPRFLEELLSPDPQMDFLA |
| KLF6-TAD | 71 | DVLPMCSIFQELQIVHETGYFSALPSLEEYWQQTCLELERYLQSEP |
| | | CYVSASEIKFDSQ |
| “C3orf62-TAD”; or | 110 | QDSPFKEEAWALLMDKSPQKATDADPGSLKQAFDDHNIVETVLDL |
| “miniC3orf62” | | EEDYNVMTSFKYQIEGSS |
| SOX7 C-term | 111 | LGQLSPPPEHPGFDALDQLSQVELLGDMDRNEFDQYLNTPGHPD |
| | | SATGAMALSGHVPVSQV |
| FOXM1 (697-762) | 112 | DVPKPGSPEPQVSGLAANRSLTEGLVLDTMNDSLSKILLDISFPGL |
| | | DEDPLGPDNINWSQFIPELQ |
| ATF6 | 113 | HRLDEDWDSALFAELGYFTDTDELQLEAANETYENNFDNLDFDLD |
| | | LVPWESDIWDINNQIGSS |
| “SERTAD2_NAHL” | 89 | SEAGTQKLDGPQESRADDSKLMDSLPGNFEITTSTGFLTDLTLDDI |
| | | LFADIDTSMYDFDP |
| “DDIT3-1”; or | 51 | MAAESLPFSFGTLSSWELEAWYEDLQEVLSSDENGGTYVSPPGN |
| “miniDDIT3” | | EEEESKIFTTLDPASL |
| ELF4 | 54 | DDIHQLEDPSVFPAVIVEQVPYPDLLHLYSGLELDDVHNGIITDGTL |
| | | CMTQDQILEGSFL |
| C11orf74 | 114 | SAHMSGLEIMDEDQLIKDVLDKFLNCHEQTYDEEFLNTFTHLSQDL |
| | | LLLPGEVEQDVST |
| “miniATMIN” | 32 | NPGPDTQLPSGPAQNPGIDFDIEEFFSASNIQTQTEESELSTMTTE |
| | | PVLESLDIETQTDF |
| “miniZXDC” | 115 | YLESGGSARTDYRAIQLAKEKKQRGAGSNAGASQSTQRKI |
| “SPDYE4”; or “S” | 90 | DPSPQPQSLGLKRKSEWSDESEEELEEELELERAPEPEDTWVVE |
| | | TLCGLKMKLKRKRASS |
| “CITED1” or “C” | 47 | ESLSPSAGAQSPAIIDSDPVDEEVLMSLVVELGLDRANELPELWLG |
| | | QNEFDFTADFPSSC |
| “RELA (p65)”; | 116 | QYLPDTDDRHRIEEKRKRTYETFKSIMKKSPFSGPTDPRPPPRRIA |
| “RELA” or “p65” | | VPSRSSASVPKPAPQPYPFTSSLSTINYDEFPTMVFPSGQISQASA |
| | | LAPAPPQVLPQAPAPAPAPAMVSALAQAPAPVPVLAPGPPQAVAP |
| | | PAPKPTQAGEGTLSEALLQLQFDDEDLGALLGNSTDPAVFTDLAS |
| | | VDNSEFQQLLNQGIPVAPHTTEPMLMEYPEAITRLVTGAQRPPDP |
| | | APAPLGAPGLPNGLLSGDEDFSSIADMDFSALL |
| “p65-AD”; | 117 | PTQAGEGTLSEALLQLQFDDEDLGALLGNSTDPAVFTDLASVDNS |
| “P_short”; or “P” | | EFQQLLNQGIPVAPHTTEPMLMEYPEAITRLVTGAQRPPDPAPAPL |
| | | GAPGLPNGLLSGDEDFSSIADMDFSALL |
| “HSF1-TAD”; | 118 | GFSVDTSALLDLFSPSVTVPDMSLPDLDSSLASIQELLSPQEPPRP |
| “H_TAD”; “HSF1” | | PEAENSSPDSGKQLVHYTAQPLFLLDPGSVDTGSNDLPVLFELGE |
| or “H” | | GSYFSEGDGFAEDPTISLLTGSEPPKAKDPTVS |
| “mini-HSF1 (401- | 119 | MLSSHGFSVDTSALLDLFSP |
| 420)” or “H(mini)” | | |
| CBX2 (CBX-C) | 136 | SASPPSTGQNPSVSVQTSQDWKPTRSLIEHVFVTDVTANLITVTVK |
| | | ESPTSVGFFNLRHY |
| CBX6 (CBX-C) | 138 | GASSEPEAGDWRPEMSPCSNVVVTDVTSNLLTVTIKEFCNPEDFE |
| | | KVAAGVAGAAGGGGSIGASK |
| CBX8 (CBX-C) | 139 | RPSLIARIPVARILGDPEEESWSPSLTNLEKVVVTDVTSNFLTVTIKE |
| | | SNTDQGFFKEKR |
| RYBP-C | 140 | IQSANATTKTSETNHTSRPRLKNVDRSTAQQLAVTVGNVTVIITDFK |
| (YAF2_RYBP) | | EKTRSSSTSSSTVTSSAGSEQQNQS |
| DDIT3_MT | 156 | SFGTLSSWELEAWYEDLQEVLSSDENGGTYVSPPGNEEEESKIFT |
| | | TLDPASLAWLT |
| SPDYE4_MT | 157 | ERAPEPEDTWVVETLCGLKMKLKRKRASS |
| ZXDC_MT | 158 | TGNFYLESGGSARTDYRAIQLAKEKKQRGAGSNAGASQSTQRKI |
| HSF1_MT | 159 | GFSVDTSALLDLFSPSVTVPDMSLPDLDSSLASIQELLSPQEPPR |
| SERTAD2_MT | 160 | LMDSLPGNFEITTSTGFLTDLTLDDILFADIDTSMYDFDP |
| ZNF473 | 161 | MAEEFVTLKDVGMDFTLGDWEQLGLEQGDTFWDTALDNCQDLFL |
| | | LDPPRPNLTSHPDGSEDLEPLAGGSPEATS |
| CITED1_MT | 162 | AIIDSDPVDEEVLMSLVVELGLDRANELPELWLGQNEFDFTADF |
| ZNF496 | 163 | MQQVTTLQLPPSRVSPFKDMILCFSEEDWSLLDPAQTGFYGEFIIG |
| | | EDYGVSMPPNDLAAQPDLSQGEENEPRVPELQDLQGKE |
| CITED2_MT | 164 | VIDTDFIDEEVLMSLVIEMGLDRIKELPELWLGQNEFDFMTDFV |
| mini-p65_MT | 165 | PTQAGEGTLSEALLQLQFDDEDLGALLGNSTDPAVFTDLASVDNS |
| | | EFQQLLNQG |
| C3orf62-2 | 166 | ATDADPGSLKQAFDDHNIVETVLDLEEDYNVMT |
| C3orf62-3 | 167 | DDHNIVETVLDLEEDYNVMT |
| miniC3orf62_1 | 168 | DADPGSLKQAFDDHNIVETVLDLEED |
| miniC3orf62_2 | 169 | DSPFKEEAWALLMDKSPQKATDADPGSL |
| miniDDIT3_1 | 170 | DLQEVLSSDENGGTYVSPPGNEEEESKIFTTLD |
| miniDDIT3_2 | 171 | LSSWELEAWYEDLQEVLSSDE |
| miniC3orf62_MT | 172 | ATDADPGSLKQAFDDHNIVETVLDLEEDYNVMTSFKYQIEGSS |
| miniSPDYE4_1 | 185 | ERAPEPEDTWVVETL |

TABLE 4
Subset of fragments identified as TADs by TADseq from Table 1.

| Gene | Fragment | Sequence | SEQ ID NO: |
|---|---|---|---|
| ATMIN | ATMIN-24 | NPGPDTQLPSGPAQNPGIDFDIEEFFSASNIQTQTEE | 32 |
| | | SELSTMTTEPVLESLDIETQTDF | |
| ATMIN | ATMIN-32 | ETQTMSSGFETLGSLFFTSNETQTAMDDFLLADLAW | 33 |
| | | NTMESQFSSVETQTSAEPHTVSNF | |
| ATOH1 | ATOH1-1 | MSRLLHAEEWAEVKELGDHHRQPQPHHLPQPPPPP | 34 |
| | | QPPATLQAREHPVYPPELSLLDSTD | |
| ATXN7L3 | ATXN7L3- | MKMEEMSLSGLDNSKLEAIAQEIYADLVEDSCLGFCF | 35 |
| | 1 | EVHRAVKCGYFFLDDTDPDSMKD | |
| ATXN7L3 | ATXN7L3- | QEIYADLVEDSCLGFCFEVHRAVKCGYFFLDDTDPDS | 36 |
| | 2 | MKDFEIVDQPGLDIFGQVFNQWK | |
| ATXN7L3 | ATXN7L3- | FEIVDQPGLDIFGQVFNQWKSKECVCPNCSRSIAASR | 37 |
| | 4 | FAPHLEKCLGMGRNSSRIANRRI | |
| BRD8 | BRD8-23 | KEECFRSGVAEAPVGSKAPSIDGKEELDLAEKMDIAV | 38 |
| | | SYTGEELDFETVGDIIAIIEDKV | |
| BRD8 | BRD8-24 | IDGKEELDLAEKMDIAVSYTGEELDFETVGDIIAIIEDK | 39 |
| | | VDDHPEVLDVAAVEAALSFCE | |
| BRD8 | BRD8-25 | GEELDFETVGDIIAIIEDKVDDHPEVLDVAAVEAALSFC | 40 |
| | | EENDDPQSLPGPWEHPIQQER | |
| BTBD18 | BTBD18- | IDCREPYAFDTALLEQPCEAEEYRITSAAATSELEEIL | 41 |
| | 24 | DFMLCGSDIEPPIGSLESPGAE | |
| BTBD18 | BTBD18- | EEYRITSAAATSELEEILDFMLCGSDIEPPIGSLESPGA | 42 |
| | 25 | EGCRTPTYHLTETGKNWIEGE | |
| C11orf74 | C11orf74-1 | MSAHMSGLEIMDEDQLIKDVLDKFLNCHEQTYDEEFL | 43 |
| | | NTFTHLSQDLLLLPGEVEQDVST | |
| C3orf62 | C3orf62-11 | NHMCGHCQDSPFKEEAWALLMDKSPQKATDADPGS | 44 |
| | | LKQAFDDHNIVETVLDLEEDYNVMT | |
| C3orf62 | C3orf62-12 | QDSPFKEEAWALLMDKSPQKATDADPGSLKQAFDD | 45 |
| | | HNIVETVLDLEEDYNVMTSFKYQIE | |
| FAM22F | FAM22F- | PRPQRPAETNAHLPPPRPQRPAETKVPEEIPPEVVQ | 55 |
| | 19 | EYVDIMEELLGSHPGDTGEPEGQR | |
| FAM22F | FAM22F- | PAETKVPEEIPPEVVQEYVDIMEELLGSHPGDTGEPE | 56 |
| | 20 | GQREKGKVEQPQEEDGITSDPGL | |
| FAM22F | FAM22F- | EKGKVEQPQEEDGITSDPGLLSYIDKLCSQEDFVTKV | 57 |
| | 22 | EAVIHPRFLEELLSPDPQMDFLA | |
| FAM22F | FAM22F- | LSYIDKLCSQEDFVTKVEAVIHPRFLEELLSPDPQMDF | 58 |
| | 23 | LALSQELEQEEGLTLAQLVEKR | |
| FAM90A1 | FAM90A1- | RQPPHSRPCLPTAQACTMSHHPAASHDGAQPLRVL | 59 |
| | 19 | FRRLENGRWSSSLLTAPSFHSPEKP | |
| FAM90A1 | FAM90A1- | HPAASHDGAQPLRVLFRRLENGRWSSSLLTAPSFHS | 60 |
| | 20 | PEKPGAFLAQSPHVSEKSEGPCVR | |
| HOXA2 | HOXA2-4 | PGSHPRHGAGGRPKPSPAGSRGSPVPAGALQPPEY | 61 |
| | | PWMKEKKAAKKTALLPAAAAAATAA | |
| HOXA2 | HOXA2-5 | RGSPVPAGALQPPEYPWMKEKKAAKKTALLPAAAAA | 62 |
| | | ATAAATGPACLSHKESLEIADGSG | |
| HOXA2 | HOXA2-6 | KKAAKKTALLPAAAAAATAAATGPACLSHKESLEIAD | 63 |
| | | GSGGGSRRLRTAYTNTQLLELEK | |
| JAZF1 | JAZF1-1 | MTGIAAASFFSNTCRFGGCGLHFPTLADLIEHIEDNHI | 68 |
| | | DTDPRVLEKQELQQPTYVALSY | |
| LIN9 | LIN9-25 | SRLTAILLQIKCLAEGGDLNSFEFKSLTDSLNDIKSTID | 74 |
| | | ASNISCFQNNVEIHVAHIQSG | |
| MYCBP | MYCBP-1 | MAHYKAADSKREQFRRYLEKSGVLDTLTKVLVALYE | 75 |
| | | EPEKPNSALDFLKHHLGAATPENP | |
| MYCL1 | MYCL1-1 | MDYDSYQHYFYDYDCGEDFYRSTAPSEDIWKKFELV | 76 |
| | | PSPPTSPPWGLGPGAGDPAPGIGP | |
| NEUROD1 | NEUROD1- | LRRMKANARERNRMHGLNAALDNLRKVVPCYSKTQ | 78 |
| | 6 | KLSKIETLRLAKNYIWALSEILRSG | |
| NEUROG3 | NEUROG3- | RRSRRKKANDRERNRMHNLNSALDALRGVLPTFPD | 79 |
| | 5 | DAKLTKIETLRFAHNYIWALTQTLR | |
| RBPJ | RBPJ-13 | GMALPRLIIRKVDKQTALLDADDPVSQLHKCAFYLKD | 85 |
| | | TERMYLCLSQERIIQFQATPCPK | |
| SPDYE4 | SPDYE4-3 | DPSPQPQSLGLKRKSEWSDESEEELEEELELERAPE | 90 |
| | | PEDTWVVETLCGLKMKLKRKRASS | |
| SPDYE4 | SPDYE4-4 | SEEELEEELELERAPEPEDTWVVETLCGLKMKLKRK | 91 |
| | | RASSVLPEHHEAFNRLLGDPVVQK | |
| SS18L2 | SS18L2-2 | QETIQRLLEENDQLIRCIVEYQNKGRGNECVQYQHVL | 92 |
| | | HRNLIYLATIADASPTSTSKAME | |
| YAF2 | YAF2-6 | EKKDKVEKEKSEKETTSKKNSHKKTRPRLKNVDRSS | 95 |
| | | AQHLEVTVGDLTVIITDFKEKTKS | |
| YAF2 | YAF2-7 | SHKKTRPRLKNVDRSSAQHLEVTVGDLTVIITDFKEKT | 96 |
| | | KSPPASSAASADQHSQSGSSSD | |
| ZXDC | ZXDC-12 | SPAEQHGAQDTELSAGTGNFYLESGGSARTDYRAIQ | 97 |
| | | LAKEKKQRGAGSNAGASQSTQRKI | |
| ZXDC | ZXDC-13 | YLESGGSARTDYRAIQLAKEKKQRGAGSNAGASQST | 98 |
| | | QRKIKEGKMSPPHFHASQNSWLCG | |
| ZXDC | ZXDC-16 | SLVVPSGGRPGPAPAAGVQCGAQGVQVQLVQDDPS | 99 |
| | | GEGVLPSARGPATFLPFLTVDLPVY | |

TABLE 5
Subset of active fragments identified as TADs by TADseq.

| | | | | Previously |
|---|---|---|---|---|
| | | | SEQ | identified |
| Gene | Fragment | Sequence | ID NO: | TAD location |
| ATF6 | ATF6-1 | MGEPAGVAGTMESPFSPGLFHRLDEDWDS | 29 | aa 1-150 |
| | | ALFAELGYFTDTDELQLEAANETYENNFDNL | | |
| ATF6 | ATF6-2 | HRLDEDWDSALFAELGYFTDTDELQLEAANE | 30 | aa 1-150 |
| | | TYENNFDNLDFDLDLVPWESDIWDINNQI | | |
| ATF6B | ATF6B-1 | MAELMLLSEIADPTRFFTDNLLSPEDWGLQN | 31 | aa 2-86 |
| | | STLYSGLDEVAEEQTQLFRCPEQDVPFDG | | |
| CSRNP1 | CSRNP1- | DNIEAPHFPLPGLSPPGDASSCFLESLMGFS | 50 | aa 493-583 |
| | 28 | EPAAEALDPFIDSQFEDTVPASLMEPVPV | | |
| KLF6 | KLF6-1 | MDVLPMCSIFQELQIVHETGYFSALPSLEEY | 70 | aa 1-200 |
| | | WQQTCLELERYLQSEPCYVSASEIKFDSQ | | |
| KLF7 | KLF7-1 | MDVLASYSIFQELQLVHDTGYFSALPSLEET | 72 | aa 1-75 |
| | | WQQTCLELERYLQTEPRRISETFGEDLDC | | |
| KLF7 | KLF7-2 | YFSALPSLEETWQQTCLELERYLQTEPRRIS | 73 | aa 1-75 |
| | | ETFGEDLDCFLHASPPPCIEESFRRLDPL | | |

TABLE 6
Subset of fragments identified as TADs.

| Gene | Fragment | Sequence | SEQ ID NO: |
|---|---|---|---|
| ATMIN | ATMIN-24 | NPGPDTQLPSGPAQNPGIDFDIEEFFSASNIQTQTEE | 32 |
| | | SELSTMTTEPVLESLDIETQTDF | |
| ATMIN | ATMIN-32 | ETQTMSSGFETLGSLFFTSNETQTAMDDFLLADLAW | 33 |
| | | NTMESQFSSVETQTSAEPHTVSNF | |
| ATOH1 | ATOH1-1 | MSRLLHAEEWAEVKELGDHHRQPQPHHLPQPPPPP | 34 |
| | | QPPATLQAREHPVYPPELSLLDSTD | |
| ATXN7L3 | ATXN7L3- | MKMEEMSLSGLDNSKLEAIAQEIYADLVEDSCLGFCF | 35 |
| | 1 | EVHRAVKCGYFFLDDTDPDSMKD | |
| ATXN7L3 | ATXN7L3- | QEIYADLVEDSCLGFCFEVHRAVKCGYFFLDDTDPDS | 36 |
| | 2 | MKDFEIVDQPGLDIFGQVFNQWK | |
| ATXN7L3 | ATXN7L3- | FEIVDQPGLDIFGQVFNQWKSKECVCPNCSRSIAASR | 37 |
| | 4 | FAPHLEKCLGMGRNSSRIANRRI | |
| BRD8 | BRD8-23 | KEECFRSGVAEAPVGSKAPSIDGKEELDLAEKMDIAV | 38 |
| | | SYTGEELDFETVGDIIAIIEDKV | |
| BRD8 | BRD8-24 | IDGKEELDLAEKMDIAVSYTGEELDFETVGDIIAIIEDK | 39 |
| | | VDDHPEVLDVAAVEAALSFCE | |
| BRD8 | BRD8-25 | GEELDFETVGDIIAIIEDKVDDHPEVLDVAAVEAALSFC | 40 |
| | | EENDDPQSLPGPWEHPIQQER | |
| BTBD18 | BTBD18- | IDCREPYAFDTALLEQPCEAEEYRITSAAATSELEEIL | 41 |
| | 24 | DFMLCGSDIEPPIGSLESPGAE | |
| BTBD18 | BTBD18- | EEYRITSAAATSELEEILDFMLCGSDIEPPIGSLESPGA | 42 |
| | 25 | EGCRTPTYHLTETGKNWIEGE | |
| FAM22F | FAM22F- | PRPQRPAETNAHLPPPRPQRPAETKVPEEIPPEVVQ | 55 |
| | 19 | EYVDIMEELLGSHPGDTGEPEGQR | |
| FAM22F | FAM22F- | PAETKVPEEIPPEVVQEYVDIMEELLGSHPGDTGEPE | 56 |
| | 20 | GQREKGKVEQPQEEDGITSDPGL | |
| HOXA2 | HOXA2-4 | PGSHPRHGAGGRPKPSPAGSRGSPVPAGALQPPEY | 61 |
| | | PWMKEKKAAKKTALLPAAAAAATAA | |
| HOXA2 | HOXA2-5 | RGSPVPAGALQPPEYPWMKEKKAAKKTALLPAAAAA | 62 |
| | | ATAAATGPACLSHKESLEIADGSG | |
| HOXA2 | HOXA2-6 | KKAAKKTALLPAAAAAATAAATGPACLSHKESLEIAD | 63 |
| | | GSGGGSRRLRTAYTNTQLLELEK | |
| JAZF1 | JAZF1-1 | MTGIAAASFFSNTCRFGGCGLHFPTLADLIEHIEDNHI | 68 |
| | | DTDPRVLEKQELQQPTYVALSY | |
| LIN9 | LIN9-25 | SRLTAILLQIKCLAEGGDLNSFEFKSLTDSLNDIKSTID | 74 |
| | | ASNISCFQNNVEIHVAHIQSG | |
| MYCBP | MYCBP-1 | MAHYKAADSKREQFRRYLEKSGVLDTLTKVLVALYE | 75 |
| | | EPEKPNSALDFLKHHLGAATPENP | |
| MYCL1 | MYCL1-1 | MDYDSYQHYFYDYDCGEDFYRSTAPSEDIWKKFELV | 76 |
| | | PSPPTSPPWGLGPGAGDPAPGIGP | |
| NEUROD1 | NEUROD1- | LRRMKANARERNRMHGLNAALDNLRKVVPCYSKTQ | 78 |
| | 6 | KLSKIETLRLAKNYIWALSEILRSG | |
| NEUROG3 | NEUROG3- | RRSRRKKANDRERNRMHNLNSALDALRGVLPTFPD | 79 |
| | 5 | DAKLTKIETLRFAHNYIWALTQTLR | |
| RBPJ | RBPJ-13 | GMALPRLIIRKVDKQTALLDADDPVSQLHKCAFYLKD | 85 |
| | | TERMYLCLSQERIIQFQATPCPK | |
| SS18L2 | SS18L2-2 | QETIQRLLEENDQLIRCIVEYQNKGRGNECVQYQHVL | 92 |
| | | HRNLIYLATIADASPTSTSKAME | |
| YAF2 | YAF2-6 | EKKDKVEKEKSEKETTSKKNSHKKTRPRLKNVDRSS | 95 |
| | | AQHLEVTVGDLTVIITDFKEKTKS | |
| YAF2 | YAF2-7 | SHKKTRPRLKNVDRSSAQHLEVTVGDLTVIITDFKEKT | 96 |
| | | KSPPASSAASADQHSQSGSSSD | |
| ZXDC | ZXDC-12 | SPAEQHGAQDTELSAGTGNFYLESGGSARTDYRAIQ | 97 |
| | | LAKEKKQRGAGSNAGASQSTQRKI | |
| ZXDC | ZXDC-13 | YLESGGSARTDYRAIQLAKEKKQRGAGSNAGASQST | 98 |
| | | QRKIKEGKMSPPHFHASQNSWLCG | |
| ZXDC | ZXDC-16 | SLVVPSGGRPGPAPAAGVQCGAQGVQVQLVQDDPS | 99 |
| | | GEGVLPSARGPATFLPFLTVDLPVY | |

SPDYE4-CITED1-RELA-HSF1 nucleotide sequence
(SEQ ID NO: 120)
SPDYE4-CITED1-RELA-HSF1 protein sequence
(SEQ ID NO: 121)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSQYLPDT
DDRHRIEEKRKRTYETFKSIMKKSPFSGPTDPRPPPRRIAVPSRSSASVPKPAPQPYPFTSSLSTINYDEFPTMVFPSG
QISQASALAPAPPQVLPQAPAPAPAPAMVSALAQAPAPVPVLAPGPPQAVAPPAPKPTQAGEGTLSEALLQLQF
DDEDLGALLGNSTDPAVFTDLASVDNSEFQQLLNQGIPVAPHTTEPMLMEYPEAITRLVTGAQRPPDPAPAPLGA
PGLPNGLLSGDEDFSSIADMDFSALLGSGSGSGFSVDTSALLDLFSPSVTVPDMSLPDLDSSLASIQELLSPQEPPRP
PEAENSSPDSGKQLVHYTAQPLFLLDPGSVDTGSNDLPVLFELGEGSYFSEGDGFAEDPTISLLTGSEPPKAKDPTVS
linker and NLS sequences are indicated with underlining
SPDYE4-CITED1-RELA nucleotide sequence
(SEQ ID NO: 122)
SPDYE4-CITED1-RELA protein sequence
(SEQ ID NO: 123)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSQYLPDT
DDRHRIEEKRKRTYETFKSIMKKSPFSGPTDPRPPPRRIAVPSRSSASVPKPAPQPYPFTSSLSTINYDEFPTMVFPSG
QISQASALAPAPPQVLPQAPAPAPAPAMVSALAQAPAPVPVLAPGPPQAVAPPAPKPTQAGEGTLSEALLQLQFD
DEDLGALLGNSTDPAVFTDLASVDNSEFQQLLNQGIPVAPHTTEPMLMEYPEAITRLVTGAQRPPDPAPAPLGAP
GLPNGLLSGDEDFSSIADMDFSALL
linker and NLS sequences are indicated with underlining
HSF1-RELA-SPDYE4-CITED1 nucleotide sequence
(SEQ ID NO: 124)
HSF1-RELA-SPDYE4-CITED1 protein sequence
(SEQ ID NO: 125)
MGFSVDTSALLDLFSPSVTVPDMSLPDLDSSLASIQELLSPQEPPRPPEAENSSPDSGKQLVHYTAQPLFLLDPGSVD
TGSNDLPVLFELGEGSYFSEGDGFAEDPTISLLTGSEPPKAKDPTVSINSRSSGSPKKKRKVGSQYLPDTDDRHRIEEK
RKRTYETFKSIMKKSPFSGPTDPRPPPRRIAVPSRSSASVPKPAPQPYPFTSSLSTINYDEFPTMVFPSGQISQASALA
PAPPQVLPQAPAPAPAPAMVSALAQAPAPVPVLAPGPPQAVAPPAPKPTQAGEGTLSEALLQLQFDDEDLGALL
GNSTDPAVFTDLASVDNSEFQQLLNQGIPVAPHTTEPMLMEYPEAITRLVTGAQRPPDPAPAPLGAPGLPNGLLS
GDEDFSSIADMDFSALLGSGSGSDPSPQPQSLGLKRKSEWSDESEEELEEELELERAPEPEDTWVVETLCGLKMKLK
RKRASSGGSGGSESLSPSAGAQSPAIIDSDPVDEEVLMSLVVELGLDRANELPELWLGQNEFDFTADFPSSCGGS
linker and NLS sequences are indicated with underlining
SPDYE4-CITED1-p65-miniHSF1 nucleotide sequence
(SEQ ID NO: 126)
SPDYE4-CITED1-p65-miniHSF1 protein sequence
(SEQ ID NO: 127)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSPTQAGE
GTLSEALLQLQFDDEDLGALLGNSTDPAVFTDLASVDNSEFQQLLNQGIPVAPHTTEPMLMEYPEAITRLVTGAQR
PPDPAPAPLGAPGLPNGLLSGDEDFSSIADMDFSALLGSGSGSMLSSHGFSVDTSALLDLFSP
linker and NLS sequences are indicated with underlining
miniSPDYE4-CITED1-p65-HSF1 nucleotide sequence
(SEQ ID NO: 128)
miniSPDYE4-CITED1-p65-HSF1 protein sequence
(SEQ ID NO: 129)
MSDESEEELEEELELERAPEPEDTWVVETLSGGSGGSESLSPSAGAQSPAIIDSDPVDEEVLMSLVVELGLDRANELP
ELWLGQNEFDFTADFPSSCGGSINSRSSGSPKKKRKVGSPTQAGEGTLSEALLQLQFDDEDLGALLGNSTDPAVFT
DLASVDNSEFQQLLNQGIPVAPHTTEPMLMEYPEAITRLVTGAQRPPDPAPAPLGAPGLPNGLLSGDEDFSSIADM
DFSALLGSGSGSGFSVDTSALLDLFSPSVTVPDMSLPDLDSSLASIQELLSPQEPPRPPEAENSSPDSGKQLVHYTAQ
PLFLLDPGSVDTGSNDLPVLFELGEGSYFSEGDGFAEDPTISLLTGSEPPKAKDPTVS
linker and NLS sequences are indicated with underlining
SPDYE4-miniCITED1-p65-HSF1 nucleotide sequence
(SEQ ID NO: 130)
SPDYE4-miniCITED1-p65-HSF1 protein sequence
(SEQ ID NO: 131)
MDPSPQPQSLGLKRKSEWSDESEEELEEELELERAPEPEDTWVVETLCGLKMKLKRKRASSGGSGGS
DSDPVDEEVLMSLVVELGLDRANELGGSINSRSSGSPKKKRKVGSPTQAGEGTLSEALLQLQFDDEDLGALLGNST
DPAVFTDLASVDNSEFQQLLNQGIPVAPHTTEPMLMEYPEAITRLVTGAQRPPDPAPAPLGAPGLPNGLLSGDED
FSSIADMDFSALLGSGSGSGFSVDTSALLDLFSPSVTVPDMSLPDLDSSLASIQELLSPQEPPRPPEAENSSPDSGKQL
VHYTAQPLFLLDPGSVDTGSNDLPVLFELGEGSYFSEGDGFAEDPTISLLTGSEPPKAKDPTVS
linker and NLS sequences are indicated with underlining
SPDYE4-CITED1-minip65(C)-HSF1 nucleotide sequence
(SEQ ID NO: 132)
SPDYE4-CITED1-minip65(C)-HSF1 protein sequence
(SEQ ID NO: 133)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSAPGLPN
GLLSGDEDFSSIADMDFSALLGSGSGSGFSVDTSALLDLFSPSVTVPDMSLPDLDSSLASIQELLSPQEPPRPPEAENS
SPDSGKQLVHYTAQPLFLLDPGSVDTGSNDLPVLFELGEGSYFSEGDGFAEDPTISLLTGSEPPKAKDPTVS
linker and NLS sequences are indicated with underlining
SPDYE4-CITED1-minip65(N)-HSF1 nucleotide sequence
(SEQ ID NO: 134)
SPDYE4-CITED1-minip65(N)-HSF1 protein sequence
(SEQ ID NO: 135)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSPTQAGE
GTLSEALLQLQFDDEDLGALLGNSTDPAVFTDLASVDNSEFQQGSGSGSGFSVDTSALLDLFSPSVTVPDMSLPDLD
SSLASIQELLSPQEPPRPPEAENSSPDSGKQLVHYTAQPLFLLDPGSVDTGSNDLPVLFELGEGSYFSEGDGFAEDPTI
SLLTGSEPPKAKDPTVS
linker and NLS sequences are indicated with underlining
>S-C3orf62.2-P_AD-H nucleotide sequence
(SEQ ID NO: 173)
>S-C3orf62.2-P_AD-H protein sequence
(SEQ ID NO: 174)
MDPSPQPQSLGLKRKSEWSDESEEELEEELELERAPEPEDTWVVETLCGLKMKLKRKRASSGGSATDADPGSLKQA
FDDHNIVETVLDLEEDYNVMTGGSINSRSSGSPKKKRKVGSPTQAGEGTLSEALLQLQFDDEDLGALLGNSTDPAV
FTDLASVDNSEFQQLLNQGIPVAPHTTEPMLMEYPEAITRLVTGAQRPPDPAPAPLGAPGLPNGLLSGDEDFSSIA
DMDFSALLGSGSGSGFSVDTSALLDLFSPSVTVPDMSLPDLDSSLASIQELLSPQEPPRPPEAENSSPDSGKQLVHYT
AQPLFLLDPGSVDTGSNDLPVLFELGEGSYFSEGDGFAEDPTISLLTGSEPPKAKDPTVS
linker and NLS sequences are indicated with underlining
>S-C3orf62.3-P_AD-H nucleotide sequence
(SEQ ID NO: 175)
>S-C3orf62.3-P_AD-H protein sequence
(SEQ ID NO: 176)
MDPSPQPQSLGLKRKSEWSDESEEELEEELELERAPEPEDTWVVETLCGLKMKLKRKRASSGGSDDHNIVETVLDL
EEDYNVMTGGSINSRSSGSPKKKRKVGSPTQAGEGTLSEALLQLQFDDEDLGALLGNSTDPAVFTDLASVDNSEFQ
QLLNQGIPVAPHTTEPMLMEYPEAITRLVTGAQRPPDPAPAPLGAPGLPNGLLSGDEDFSSIADMDFSALLGSGSG
SGFSVDTSALLDLFSPSVTVPDMSLPDLDSSLASIQELLSPQEPPRPPEAENSSPDSGKQLVHYTAQPLFLLDPGSVDT
GSNDLPVLFELGEGSYFSEGDGFAEDPTISLLTGSEPPKAKDPTVS
linker and NLS sequences are indicated with underlining
>S-C3orf62_MT-P_AD-H nucleotide sequence
(SEQ ID NO: 177)
>S-C3orf62_MT-P_AD-H protein sequence
(SEQ ID NO: 178)
MDPSPQPQSLGLKRKSEWSDESEEELEEELELERAPEPEDTWVVETLCGLKMKLKRKRASSGGSATDADPGSLKQA
FDDHNIVETVLDLEEDYNVMTSFKYQIEGSSGGSINSRSSGSPKKKRKVGSPTQAGEGTLSEALLQLQFDDEDLGAL
LGNSTDPAVFTDLASVDNSEFQQLLNQGIPVAPHTTEPMLMEYPEAITRLVTGAQRPPDPAPAPLGAPGLPNGLLS
GDEDFSSIADMDFSALLGSGSGSGFSVDTSALLDLFSPSVTVPDMSLPDLDSSLASIQELLSPQEPPRPPEAENSSPD
SGKQLVHYTAQPLFLLDPGSVDTGSNDLPVLFELGEGSYFSEGDGFAEDPTISLLTGSEPPKAKDPTVS
linker and NLS sequences are indicated with underlining
>S-DDIT3_MT-P_AD-H nucleotide sequence
(SEQ ID NO: 179)
>S-DDIT3_MT-P_AD-H protein sequence
(SEQ ID NO: 180)
MDPSPQPQSLGLKRKSEWSDESEEELEEELELERAPEPEDTWVVETLCGLKMKLKRKRASSGGSGGSSFGTLSSWE
LEAWYEDLQEVLSSDENGGTYVSPPGNEEEESKIFTTLDPASLAWLTGGSINSRSSGSPKKKRKVGSPTQAGEGTLS
EALLQLQFDDEDLGALLGNSTDPAVFTDLASVDNSEFQQLLNQGIPVAPHTTEPMLMEYPEAITRLVTGAQRPPDP
APAPLGAPGLPNGLLSGDEDFSSIADMDFSALLGSGSGSGFSVDTSALLDLFSPSVTVPDMSLPDLDSSLASIQELLS
PQEPPRPPEAENSSPDSGKQLVHYTAQPLFLLDPGSVDTGSNDLPVLFELGEGSYFSEGDGFAEDPTISLLTGSEPPK
AKDPTVS
linker and NLS sequences are indicated with underlining
>S-C-P_AD-HSF1_MT nucleotide sequence
(SEQ ID NO: 181)
>S-C-P_AD-HSF1_MT protein sequence
(SEQ ID NO: 182)
MDPSPQPQSLGLKRKSEWSDESEEELEEELELERAPEPEDTWVVETLCGLKMKLKRKRASSGGSGGSESLSPSAGA
QSPAIIDSDPVDEEVLMSLVVELGLDRANELPELWLGQNEFDFTADFPSSCGGSINSRSSGSPKKKRKVGSPTQAGE
GTLSEALLQLQFDDEDLGALLGNSTDPAVFTDLASVDNSEFQQLLNQGIPVAPHTTEPMLMEYPEAITRLVTGAQR
PPDPAPAPLGAPGLPNGLLSGDEDFSSIADMDFSALLGSGSGSGFSVDTSALLDLFSPSVTVPDMSLPDLDSSLASIQ
ELLSPQEPPR
linker and NLS sequences are indicated with underlining
>3 x ZNF473_KRAB nucleotide sequence
(SEQ ID NO: 183)
>3 x ZNF473_KRAB protein sequence
(SEQ ID NO: 184)
MAEEFVTLKDVGMDFTLGDWEQLGLEQGDTFWDTALDNCQDLFLLDPPRPNLTSHPDGSEDLEPLAGGSPEATS
MAEEFVTLKDVGMDFTLGDWEQLGLEQGDTFWDTALDNCQDLFLLDPPRPNLTSHPDGSEDLEPLAGGSPEATS
MAEEFVTLKDVGMDFTLGDWEQLGLEQGDTFWDTALDNCQDLFLLDPPRPNLTSHPDGSEDLEPLAGGSPEATS

### REFERENCES

Adusumilli, R., and Mallick, P. (2017). Data Conversion with ProteoWizard msConvert. Methods Mol. Biol. Clifton NJ 1550, 339-368.
Alekseyenko, A. A., Gorchakov, A. A., Kharchenko, P. V., and Kuroda, M. I. (2014). Reciprocal interactions of human C10orf12 and C17orf96 with PRC2 revealed by BioTAP-XL cross-linking and affinity purification. Proc. Natl. Acad. Sci. 111, 2488-2493.
Ali, R. H., and Rouzbahman, M. (2015). Endometrial stromal tumours revisited: an update based on the 2014 WHO classification. J. Clin. Pathol. 68, 325-332.
Alonso, C. N., Meyer, C., Gallego, M. S., Rossi, J. G., Mansini, A. P., Rubio, P. L., Medina, A., Marschalek, R., and Felice, M. S. (2010). BTBD18: A novel MLL partner gene in an infant with acute lymphoblastic leukemia and inv(11)(q13;q23). Leuk. Res. 34, e294-e296.
Antonescu, C., Sung, Y.-S., Zhang, L., Agaram, N., and Fletcher, C. (2017). Recurrent SRF-RELA Fusions Define a Novel Subset of Cellular Myofibroma/Myopericytoma: A Potential Diagnostic Pitfall With Sarcomas With Myogenic Differentiation. Am. J. Surg. Pathol. 41, 677-684.
Arnold, C. D., Nemčko, F., Woodfin, A. R., Wienerroither, S., Vlasova, A., Schleiffer, A., Pagani, M., Rath, M., and Stark, A. (2018). A high-throughput method to identify trans-activation domains within transcription factor sequences. EMBO J. 37.
Badis, G., Berger, M. F., Philippakis, A. A., Talukder, S., Gehrke, A. R., Jaeger, S. A., Chan, E. T., Metzler, G., Vedenko, A., Chen, X., et al. (2009). Diversity and Complexity in DNA Recognition by Transcription Factors. Science 324, 1720-1723.
Barreto, G., Schafer, A., Marhold, J., Stach, D., Swaminathan, S. K., Handa, V., DOderlein, G., Maltry, N., Wu, W., Lyko, F., et al. (2007). Gadd45a promotes epigenetic gene activation by repair-mediated DNA demethylation. Nature 445, 671-675.
Barrett, J., Birrer, M. J., Kato, G. J., Dosaka-Akita, H., and Dang, C. V. (1992). Activation domains of L-Myc and c-Myc determine their transforming potencies in rat embryo cells. Mol. Cell. Biol. 12, 3130-3137.
Basnet, H., Su, X. B., Tan, Y., Meisenhelder, J., Merkurjev, D., Ohgi, K. A., Hunter, T., Pillus, L., and Rosenfeld, M. G. (2014). Tyrosine phosphorylation of histone H2A by CK2 regulates transcriptional elongation. Nature 516, 267-271.
Basu, S., Mackowiak, S. D., Niskanen, H., Knezevic, D., Asimi, V., Grosswendt, S., Geertsema, H., Ali, S., Jerković, I., Ewers, H., et al. (2020). Unblending of Transcriptional Condensates in Human Repeat Expansion Disease. Cell 181, 1062-1079.e30.
Benjamini, Y., Krieger, A. M., and Yekutieli, D. (2006). Adaptive linear step-up procedures that control the false discovery rate. Biometrika 93, 491-507.
Bousoik, E., and Montazeri Aliabadi, H. (2018). “Do We Know Jack” About JAK? A Closer Look at JAK/STAT Signaling Pathway. Front. Oncol. 8.
Breitkreutz, A., Choi, H., Sharom, J. R., Boucher, L., Neduva, V., Larsen, B., Lin, Z.-Y., Breitkreutz, B.-J., Stark, C., Liu, G., et al. (2010). A global protein kinase and phosphatase interaction network in yeast. Sci. N. Y. NY 328, 1043-1046.
Brien, G. L., Remillard, D., Shi, J., Hemming, M. L., Chabon, J., Wynne, K., Dillon, E. T., Cagney, G., Van Mierlo, G., Baltissen, M. P., et al. (2018). Targeted degradation of BRD9 reverses oncogenic gene expression in synovial sarcoma. ELife 7, e41305.
Buchwalter, G., Gross, C., and Wasylyk, B. (2004). Ets ternary complex transcription factors. Gene 324, 1-14.
Cao, L., Yonis, A., Vaghela, M., Barriga, E. H., Chugh, P., Smith, M. B., Maufront, J., Lavoie, G., Meant, A., Ferber, E., et al. (2020). SPIN90 associates with mDia1 and the Arp2/3 complex to regulate cortical actin organization. Nat. Cell Biol. 22, 803-814.
Centore, R. C., Sandoval, G. J., Soares, L. M. M., Kadoch, C., and Chan, H. M. (2020). Mammalian SWI/SNF Chromatin Remodeling Complexes: Emerging Mechanisms and Therapeutic Strategies. Trends Genet. 36, 936-950.
Chauhan, S., Zheng, X., Tan, Y. Y., Tay, B.-H., Lim, S., Venkatesh, B., and Kaldis, P. (2012). Evolution of the Cdk-activator Speedy/RINGO in vertebrates. Cell. Mol. Life Sci. 69, 3835-3850.
Chavez, A., Scheiman, J., Vora, S., Pruitt, B. W., Tuttle, M., lyer, E. P. R., Lin, S., Kiani, S., Guzman, C. D., Wiegand, D. J., et al. (2015). Highly efficient Cas9-mediated transcriptional programming. Nat. Methods 12, 326-328.
Chavez, A., Tuttle, M., Pruitt, B. W., Ewen-Campen, B., Chari, R., Ter-Ovanesyan, D., Haque, S. J., Cecchi, R. J., Kowal, E. J. K., Buchthal, J., et al. (2016). Comparison of Cas9 activators in multiple species. Nat. Methods 13, 563-567.
Chen, F. X., Smith, E. R., and Shilatifard, A. (2018). Born to run: control of transcription elongation by RNA polymerase II. Nat. Rev. Mol. Cell Biol. 19, 464-478.
Conaway, R. C., and Conaway, J. W. (2011). Function and regulation of the Mediator complex. Curr. Opin. Genet. Dev. 21, 225-230.
Core, L., and Adelman, K. (2019). Promoter-proximal pausing of RNA polymerase II: a nexus of gene regulation. Genes Dev. 33, 960-982.
Cramer, P. (2019). Organization and regulation of gene transcription. Nature 573, 45-54.
Davis, R. L., Weintraub, H., and Lassar, A. B. (1987). Expression of a single transfected cDNA converts fibroblasts to myoblasts. Cell 51, 987-1000.
Di Rocco, G., Mavilio, F., and Zappavigna, V. (1997). Functional dissection of a transcriptionally active, target-specific Hox-Pbx complex. EMBO J. 16, 3644-3654.
Dyson, H. J., and Wright, P. E. (2005). Intrinsically unstructured proteins and their functions. Nat. Rev. Mol. Cell Biol. 6, 197-208.
Eng, J. K., Jahan, T. A., and Hoopmann, M. R. (2013). Comet: an open-source MS/MS sequence database search tool. PROTEOMICS 13, 22-24.
Erijman, A., Kozlowski, L., Sohrabi-Jahromi, S., Fishburn, J., Warfield, L., Schreiber, J., Noble, W. S., Söding, J., and Hahn, S. (2020). A High-Throughput Screen for Transcription Activation Domains Reveals Their Sequence Features and Permits Prediction by Deep Learning. Mol. Cell 78, 890-902.e6.
Esnault, C., Stewart, A., Gualdrini, F., East, P., Horswell, S., Matthews, N., and Treisman, R. (2014). Rho-actin signaling to the MRTF coactivators dominates the immediate transcriptional response to serum in fibroblasts. Genes Dev. 28, 943-958.
Fujisawa, T., and Filippakopoulos, P. (2017). Functions of bromodomain-containing proteins and their roles in homeostasis and cancer. Nat. Rev. Mol. Cell Biol. 18, 246-262.
Gahan, J. M., Rentzsch, F., and Schnitzler, C. E. (2020). The genetic basis for PRC1 complex diversity emerged early in animal evolution. Proc. Natl. Acad. Sci. 117, 22880-22889.
Gao, Y., Xiong, X., Wong, S., Charles, E. J., Lim, W. A., and Qi, L. S. (2016). Complex transcriptional modulation with orthogonal and inducible dCas9 regulators. Nat. Methods 13, 1043-1049.
Gao, Z., Zhang, J., Bonasio, R., Strino, F., Sawai, A., Parisi, F., Kluger, Y., and Reinberg, D. (2012). PCGF Homologs, CBX Proteins, and RYBP Define Functionally Distinct PRC1 Family Complexes. Mol. Cell 45, 344-356.
Gao, Z., Lee, P., Stafford, J. M., von Schimmelmann, M., Schaefer, A., and Reinberg, D. (2014). An AUTS2-Polycomb complex activates gene expression in the CNS. Nature 516, 349-354.
Gastwirt, R. F., McAndrew, C. W., and Donoghue, D. J. (2007). Speedy/RINGO Regulation of CDKs in Cell Cycle, Checkpoint Activation and Apoptosis. Cell Cycle 6, 1188-1193.
Gerritsen, M. E., Williams, A. J., Neish, A. S., Moore, S., Shi, Y., and Collins, T. (1997). CREB-binding protein/p300 are transcriptional coactivators of p65. Proc. Natl. Acad. Sci. U.S.A. 94, 2927-2932.
Gonzalez, L., and Nebreda, A. R. (2020). RINGO/Speedy proteins, a family of non-canonical activators of CDK1 and CDK2. Semin. Cell Dev. Biol. 107, 21-27.
Goparaju, S. K., Kohda, K., Ibata, K., Soma, A., Nakatake, Y., Akiyama, T., Wakabayashi, S.,
Matsushita, M., Sakota, M., Kimura, H., et al. (2017). Rapid differentiation of human pluripotent stem cells into functional neurons by mRNAs encoding transcription factors. Sci. Rep. 7, 42367.
Gualdrini, F., Esnault, C., Horswell, S., Stewart, A., Matthews, N., and Treisman, R. (2016). SRF Co-factors Control the Balance between Cell Proliferation and Contractility. Mol. Cell 64, 1048-1061.
Guo, Z., Zhang, L., Wu, Z., Chen, Y., Wang, F., and Chen, G. (2014). In vivo direct reprogramming of reactive glial cells into functional neurons after brain injury and in an Alzheimer's disease model. Cell Stem Cell 14, 188-202.
Haberle, V., Arnold, C. D., Pagani, M., Rath, M., Schernhuber, K., and Stark, A. (2019). Transcriptional cofactors display specificity for distinct types of core promoters. Nature 570, 122-126.
Hirai, H., Tani, T., Katoku-Kikyo, N., Kellner, S., Karian, P., Firpo, M., and Kikyo, N. (2011). Radical acceleration of nuclear reprogramming by chromatin remodeling with the transactivation domain of MyoD. Stem Cells Dayt. Ohio 29, 1349-1361.
Horb, M. E., Shen, C.-N., Tosh, D., and Slack, J. M. W. (2003). Experimental Conversion of Liver to Pancreas. Curr. Biol. 13, 105-115.
Hrzenjak, A. (2016). JAZF1/SUZ12 gene fusion in endometrial stromal sarcomas. Orphanet J. Rare Dis. 11, 15.
Hsu, S. I., Yang, C. M., Sim, K. G., Hentschel, D. M., O'Leary, E., and Bonventre, J. V. (2001). TRIP-Br: a novel family of PHD zinc finger- and bromodomain-interacting proteins that regulate the transcriptional activity of E2F-1/DP-1. EMBO J. 20, 2273-2285.
Huttlin, E. L., Bruckner, R. J., Navarrete-Perea, J., Cannon, J. R., Baltier, K., Gebreab, F., Gygi, M. P., Thornock, A., Zarraga, G., Tam, S., et al. (2020). Dual Proteome-scale Networks Reveal Cell-specific Remodeling of the Human Interactome (Systems Biology).
Israni, D. V., Li, H.-S., Gagnon, K. A., Sander, J. D., Roybal, K. T., Joung, J. K., Wong, W. W., and Khalil, A. S. (2021). Clinically-driven design of synthetic gene regulatory programs in human cells (Synthetic Biology).
Jolma, A., Yan, J., Whitington, T., Toivonen, J., Nitta, K. R., Rastas, P., Morgunova, E., Enge, M., Taipale, M., Wei, G., et al. (2013). DNA-binding specificities of human transcription factors. Cell 152, 327-339.
Karanian, M., Pissaloux, D., Gomez-Brouchet, A., Chevenet, C., Le Loarer, F., Fernandez, C., Minard, V., Corradini, N., Castex, M.-P., Duc-Gallet, A., et al. (2020). SRF-FOXO1 and SRF-NCOA1 Fusion Genes Delineate a Distinctive Subset of Well-differentiated Rhabdomyosarcoma. Am. J. Surg. Pathol. 44, 607-616.
Keung, A. J., Bashor, C. J., Kiriakov, S., Collins, J. J., and Khalil, A. S. (2014). Using targeted chromatin regulators to engineer combinatorial and spatial transcriptional regulation. Cell 158, 110-120.
Kim, D. I., Jensen, S. C., Noble, K. A., Kc, B., Roux, K. H., Motamedchaboki, K., and Roux, K. J. (2016). An improved smaller biotin ligase for BiolD proximity labeling. Mol. Biol. Cell 27, 1188-1196.
Knight, J. D. R., Choi, H., Gupta, G. D., Pelletier, L., Raught, B., Nesvizhskii, A. I., and Gingras, A.-C. (2017). ProHits-viz: a suite of web tools for visualizing interaction proteomics data. Nat. Methods 14, 645-646.
Kundu, T. K., Palhan, V. B., Wang, Z., An, W., Cole, P. A., and Roeder, R. G. (2000). Activator-dependent transcription from chromatin in vitro involving targeted histone acetylation by p300. Mol. Cell 6, 551-561.
Lambert, J.-P., Tucholska, M., Go, C., Knight, J. D. R., and Gingras, A.-C. (2015). Proximity biotinylation and affinity purification are complementary approaches for the interactome mapping of chromatin-associated protein complexes. J. Proteomics 118, 81-94.
Lambert, J.-P., Picaud, S., Fujisawa, T., Hou, H., Savitsky, P., Uuskula-Reimand, L., Gupta, G. D., Abdouni, H., Lin, Z.-Y., Tucholska, M., et al. (2019). Interactome Rewiring Following Pharmacological Targeting of BET Bromodomains. Mol. Cell 73, 621-638.e17.
Lambert, S. A., Jolma, A., Campitelli, L. F., Das, P. K., Yin, Y., Albu, M., Chen, X., Taipale, J., Hughes, T. R., and Weirauch, M. T. (2018). The Human Transcription Factors. Cell 172, 650-665.
Lecoq, L., Raiola, L., Chabot, P. R., Cyr, N., Arseneault, G., Legault, P., and Omichinski, J. G. (2017). Structural characterization of interactions between transactivation domain 1 of the p65 subunit of NF-κB and transcription regulatory factors. Nucleic Acids Res. 45, 5564-5576.
Li, X., Wang, W., Wang, J., Malovannaya, A., Xi, Y., Li, W., Guerra, R., Hawke, D. H., Qin, J., and Chen, J. (2015). Proteomic analyses reveal distinct chromatin-associated and soluble transcription factor complexes. Mol. Syst. Biol. 11.
Liang, F.-S., Ho, W. Q., and Crabtree, G. R. (2011). Engineering the ABA plant stress pathway for regulation of induced proximity. Sci. Signal. 4, rs2-rs2.
Liu, G., Knight, J. D. R., Zhang, J. P., Tsou, C.-C., Wang, J., Lambert, J.-P., Larsen, B., Tyers, M., Raught, B., Bandeira, N., et al. (2016). Data Independent Acquisition analysis in ProHits 4.0. J. Proteomics 149, 64-68.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Luck, K., Kim, D.-K., Lambourne, L., Spirohn, K., Begg, B. E., Bian, W., Brignall, R., Cafarelli, T., Campos-Laborie, F. J., Charloteaux, B., et al. (2020). A reference map of the human binary protein interactome. Nature 580, 402-408.
Maeder M L, Thibodeau-Beganny S, Osiak A, Wright D A, Anthony R M, Eichtinger M, Jiang T, Foley J E, Winfrey R J, Townsend J A, Unger-Wallace E, Sander J D, Müller-Lerch F, Fu F, Pearlberg J, GObel C, Dassie J P, Pruett-Miller S M, Porteus M H, Sgroi D C, lafrate A J, Dobbs D, McCray P B Jr, Cathomen T, Voytas D F, Joung J K. Rapid “open-source” engineering of customized zinc-finger nucleases for highly efficient gene modification. Mol Cell. 2008 Jul. 25; 31(2):294-301.
Marcon, E., Ni, Z., Pu, S., Turinsky, A. L., Trimble, S. S., Olsen, J. B., Silverman-Gavrila, R., Silverman-Gavrila, L., Phanse, S., Guo, H., et al. (2014). Human-Chromatin-Related Protein Interactions Identify a Demethylase Complex Required for Chromosome Segregation. Cell Rep. 8, 297-310.
Mashtalir, N., D'Avino, A. R., Michel, B. C., Luo, J., Pan, J., Otto, J. E., Zullow, H. J., McKenzie, Z. M., Kubiak, R. L., St Pierre, R., et al. (2018). Modular Organization and Assembly of SWI/SNF Family Chromatin Remodeling Complexes. Cell 175, 1272-1288.e20.
McGrath, D. A., Fifield, B.-A., Marceau, A. H., Tripathi, S., Porter, L. A., and Rubin, S. M. (2017). Structural basis of divergent cyclin-dependent kinase activation by Spy1/RINGO proteins. EMBO J. 36, 2251-2262.
Mellacheruvu, D., Wright, Z., Couzens, A. L., Lambert, J.-P., St-Denis, N. A., Li, T., Miteva, Y. V., Hauri, S., Sardiu, M. E., Low, T. Y., et al. (2013). The CRAPome: a contaminant repository for affinity purification-mass spectrometry data. Nat. Methods 10, 730-736.
Miralles, F., Posern, G., Zaromytidou, A.-I., and Treisman, R. (2003). Actin Dynamics Control SRF Activity by Regulation of Its Coactivator MAL. Cell 113, 329-342.
Morey, L., Pascual, G., Cozzuto, L., Roma, G., Wutz, A., Benitah, S. A., and Di Croce, L. (2012). Nonoverlapping Functions of the Polycomb Group Cbx Family of Proteins in Embryonic Stem Cells. Cell Stem Cell 10, 47-62.
Morita, K., Celso, C. L., Spencer-Dene, B., Zouboulis, C. C., and Watt, F. M. (2006). HAN11 binds mDia1 and controls GLI1 transcriptional activity. J. Dermatol. Sci. 44, 11-20.
Muhar, M., Ebert, A., Neumann, T., Umkehrer, C., Jude, J., Wieshofer, C., Rescheneder, P., Lipp, J. J., Herzog, V. A., Reichholf, B., et al. (2018). SLAM-seq defines direct gene-regulatory functions of the BRD4-MYC axis. Science 360, 800-805.
Najafabadi, H. S., Mnaimneh, S., Schmitges, F. W., Garton, M., Lam, K. N., Yang, A., Albu, M., Weirauch, M. T., Radovani, E., Kim, P. M., et al. (2015). C2H2 zinc finger proteins greatly expand the human regulatory lexicon. Nat. Biotechnol. 33, 555-562.
Narayan, S., Bryant, G., Shah, S., Berrozpe, G., and Ptashne, M. (2017). OCT4 and SOX2 Work as Transcriptional Activators in Reprogramming Human Fibroblasts. Cell Rep. 20, 1585-1596.
Nasrin, N., Ogg, S., Cahill, C. M., Biggs, W., Nui, S., Dore, J., Calvo, D., Shi, Y., Ruvkun, G., and Alexander-Bridges, M. C. (2000). DAF-16 recruits the CREB-binding protein coactivator complex to the insulin-like growth factor binding protein 1 promoter in HepG2 cells. Proc. Natl. Acad. Sci. 97, 10412-10417.
Ng, A. H. M., Khoshakhlagh, P., Rojo Arias, J. E., Pasquini, G., Wang, K., Swiersy, A., Shipman, S. L., Appleton, E., Kiaee, K., Kohman, R. E., et al. (2021). A comprehensive library of human transcription factors for cell fate engineering. Nat. Biotechnol. 39, 510-519.
Olson, E. N., and Nordheim, A. (2010). Linking actin dynamics and gene transcription to drive cellular motile functions. Nat. Rev. Mol. Cell Biol. 11, 353-365.
ORFeome Collaboration (2016). The ORFeome Collaboration: a genome-scale human ORF-clone resource. Nat. Methods 13, 191-192.
Pellizzoni, L., Charroux, B., Rappsilber, J., Mann, M., and Dreyfuss, G. (2001). A Functional Interaction between the Survival Motor Neuron Complex and RNA Polymerase II. J. Cell Biol. 152, 75-86.
Piette, B. L., Alerasool, N., Lin, Z.-Y., Lacoste, J., Lam, M. H. Y., Qian, W. W., Tran, S., Larsen, B., Campos, E., Peng, J., et al. (2021). Comprehensive interactome profiling of the human Hsp70 network highlights functional differentiation of J domains. Mol. Cell 0.
Piper, D. E., Batchelor, A. H., Chang, C.-P., Cleary, M. L., and Wolberger, C. (1999). Structure of a HoxB1-Pbx1 Heterodimer Bound to DNA: Role of the Hexapeptide and a Fourth Homeodomain Helix in Complex Formation. Cell 96, 587-597.
Piskacek, S., Gregor, M., Nemethova, M., Grabner, M., Kovarik, P., and Piskacek, M. (2007). Nine-amino-acid transactivation domain: Establishment and prediction utilities. Genomics 89, 756-768.
Piunti, A., Smith, E. R., Morgan, M. A. J., Ugarenko, M., Khaltyan, N., Helmin, K. A., Ryan, C. A., Murray, D. C., Rickels, R. A., Yilmaz, B. D., et al. (2019). CATACOMB: An endogenous inducible gene that antagonizes H3K27 methylation activity of Polycomb repressive complex 2 via an H3K27M-like mechanism. Sci. Adv. 5, eaax2887.
Ptashne, M., and Gann, A. (1997). Transcriptional activation by recruitment. Nature 386, 569-577.
Ravarani, C. N., Erkina, T. Y., De Baets, G., Dudman, D. C., Erkine, A. M., and Babu, M. M. (2018). High-throughput discovery of functional disordered regions: investigation of transactivation domains. Mol. Syst. Biol. 14, e8190.
Robinson, M. D., McCarthy, D. J., and Smyth, G. K. (2010). edgeR: a Bioconductor package for differential expression analysis of digital gene expression data. Bioinforma. Oxf. Engl. 26, 139-140.
Ryseck, R. P., Bull, P., Takamiya, M., Bours, V., Siebenlist, U., Dobrzanski, P., and Bravo, R. (1992). RelB, a new Rel family transcription activator that can interact with p50-NF-kappa B. Mol. Cell. Biol. 12, 674-684.
Sadowski, I., Ma, J., Triezenberg, S., and Ptashne, M. (1988). GAL4-VP16 is an unusually potent transcriptional activator. Nature 335, 563-564.
Sanborn, A. L., Yeh, B. T., Feigerle, J. T., Hao, C. V., Townshend, R. J. L., Aiden, E. L., Dror, R. O., and Kornberg, R. D. (2020). Simple biochemical features underlie transcriptional activation domain diversity and dynamic, fuzzy binding to Mediator. BioRxiv 2020.12.18.423551.
Sanjana, N., Cong, L., Zhou, Y. et al. A transcription activator-like effector toolbox for genome engineering. Nat Protoc 7, 171-192 (2012).
Sano, K., Hayakawa, A., Piao, J.-H., Kosaka, Y., and Nakamura, H. (2000). Novel SH3 protein encoded by the AF3p21 gene is fused to the mixed lineage leukemia protein in a therapy-related leukemia with t(3;11) (p21;q23). Blood 95, 1066-1068.
Schratt, G., Philippar, U., Berger, J., Schwarz, H., Heidenreich, O., and Nordheim, A. (2002). Serum response factor is crucial for actin cytoskeletal organization and focal adhesion assembly in embryonic stem cells. J. Cell Biol. 156, 737-750.
Sdelci, S., Lardeau, C.-H., Tallant, C., Klepsch, F., Klaiber, B., Bennett, J., Rathert, P., Schuster, M., Penz, T., Fedorov, O., et al. (2016). Mapping the chemical chromatin reactivation landscape identifies BRD4-TAF1 cross-talk. Nat. Chem. Biol. 12, 504-510.
Shteynberg, D., Deutsch, E. W., Lam, H., Eng, J. K., Sun, Z., Tasman, N., Mendoza, L., Moritz, R. L., Aebersold, R., and Nesvizhskii, A. I. (2011). iProphet: multi-level integrative analysis of shotgun proteomic data improves peptide and protein identification rates and error estimates. Mol. Span Classnocasespan Cell. Proteomics MCP 10, M111.007690.
Sigler, P. B. (1988). Transcriptional activation. Acid blobs and negative noodles. Nature 333, 210-212.
Singh, R. N., Howell, M. D., Ottesen, E. W., and Singh, N. N. (2017). Diverse role of survival motor neuron protein. Biochim. Biophys. Acta BBA—Gene Regul. Mech. 1860, 299-315.
Skapek, S. X., Ferrari, A., Gupta, A. A., Lupo, P. J., Butler, E., Shipley, J., Barr, F. G., and Hawkins, D. S. (2019). Rhabdomyosarcoma. Nat. Rev. Dis. Primer 5, 1-19.
Staller, M. V., Ramirez, E., Holehouse, A. S., Pappu, R. V., and Cohen, B. A. (2021). Design principles of acidic transcriptional activation domains. BioRxiv 2020.10.28.359026.
Stampfel, G., Kazmar, T., Frank, O., Wienerroither, S., Reiter, F., and Stark, A. (2015). Transcriptional regulators form diverse groups with context-dependent regulatory functions. Nature 528, 147-151.
Strasswimmer, J., Lorson, C. L., Breiding, D. E., Chen, J. J., Le, T., Burghes, A. H. M., and Androphy, E. J. (1999). Identification of Survival Motor Neuron as a Transcriptional Activator-Binding Protein. Hum. Mol. Genet. 8, 1219-1226.
Sudarshan, D., Avvakumov, N., Lalonde, M.-E., Alerasool, N., Jacquet, K., Mameri, A., Rousseau, J., Lambert, J.-P., Paquet, E., Setty, S T., et al. (2021). Recurrent chromosomal translocations in sarcomas create a mega-complex that mislocalizes NuA4/TIP60 to Polycomb target loci. BioRxiv 2021.03.26.436670.
Tague, E. P., Dotson, H. L., Tunney, S. N. et al. Chemogenetic control of gene expression and cell signaling with antiviral drugs. Nat Methods 15, 519-522 (2018).
Taipale, M., Tucker, G., Peng, J., Krykbaeva, I., Lin, Z.-Y., Larsen, B., Choi, H., Berger, B., Gingras, A.-C., and Lindquist, S. (2014). A quantitative chaperone interaction network reveals the architecture of cellular protein homeostasis pathways. Cell 158, 434-448.
Tanenbaum M E, Gilbert L A, Qi L S, Weissman J S, Vale R D. A protein-tagging system for signal amplification in gene expression and fluorescence imaging. Cell. 2014; 159(3):635-646. doi:10.1016/j.cell.2014.09.039
Teo, G., Liu, G., Zhang, J., Nesvizhskii, A. I., Gingras, A.-C., and Choi, H. (2014). SAINTexpress: Improvements and additional features in Significance Analysis of INTeractome software. J. Proteomics 100, 37-43.
The ENCODE Project Consortium, Moore, J. E., Purcaro, M. J., Pratt, H. E., Epstein, C. B., Shoresh, N., Adrian, J., Kawli, T., Davis, C. A., Dobin, A., et al. (2020). Expanded encyclopaedias of DNA elements in the human and mouse genomes. Nature 583, 699-710.
Theodorou, E., Dalembert, G., Heffelfinger, C., White, E., Weissman, S., Corcoran, L., and Snyder, M. (2009). A high throughput embryonic stem cell screen identifies Oct-2 as a bifunctional regulator of neuronal differentiation. Genes Dev. 23, 575-588.
Tycko, J., DelRosso, N., Hess, G. T., Aradhana, Banerjee, A., Mukund, A., Van, M. V., Ego, B. K., Yao, D., Spees, K., et al. (2020). High-Throughput Discovery and Characterization of Human Transcriptional Effectors. Cell 0.
Vannam, R., Sayilgan, J., Ojeda, S., Karakyriakou, B., Hu, E., Kreuzer, J., Morris, R., Herrera Lopez, X. I., Rai, S., Haas, W., et al. (2021). Targeted degradation of the enhancer lysine acetyltransferases CBP and p300. Cell Chem. Biol. 28, 503-514.e12.
Vartiainen, M. K., Guettler, S., Larijani, B., and Treisman, R. (2007). Nuclear Actin Regulates Dynamic Subcellular Localization and Activity of the SRF Cofactor MAL. Science 316, 1749-1752.
Vihervaara, A., Duarte, F. M., and Lis, J. T. (2018). Molecular mechanisms driving transcriptional stress responses. Nat. Rev. Genet. 19, 385-397.
Vincenz, C., and Kerppola, T. K. (2008). Different polycomb group CBX family proteins associate with distinct regions of chromatin using nonhomologous protein sequences. Proc. Natl. Acad. Sci. 105, 16572-16577.
Wang, R., Ilangovan, U., Robinson, A. K., Schirf, V., Schwarz, P. M., Lafer, E. M., Demeler, B., Hinck, A. P., and Kim, C. A. (2008). Structural transitions of the RING1B C-terminal region upon binding the polycomb cbox domain. Biochemistry 47, 8007-8015.
Wang, R., Taylor, A. B., Leal, B. Z., Chadwell, L. V., Ilangovan, U., Robinson, A. K., Schirf, V., Hart, P. J., Lafer, E. M., Demeler, B., et al. (2010). Polycomb Group Targeting through Different Binding Partners of RING1B C-Terminal Domain. Structure 18, 966-975.
Wang, Y., Li, Y., Zeng, W., Zhu, C., Xiao, J., Yuan, W., Wang, Y., Cai, Z., Zhou, J., Liu, M., et al. (2004). IXL, a new subunit of the mammalian Mediator complex, functions as a transcriptional suppressor. Biochem. Biophys. Res. Commun. 325, 1330-1338.
Wang, Y., Chen, J., Hu, J.-L., Wei, X.-X., Qin, D., Gao, J., Zhang, L., Jiang, J., Li, J.-S., Liu, J., et al. (2011). Reprogramming of mouse and human somatic cells by high-performance engineered factors. EMBO Rep. 12, 373-378.
Weirauch, M. T., Yang, A., Albu, M., Cote, A. G., Montenegro-Montero, A., Drewe, P., Najafabadi, H. S., Lambert, S. A., Mann, I., Cook, K., et al. (2014). Determination and Inference of Eukaryotic Transcription Factor Sequence Specificity. Cell 158, 1431-1443.
Winters, A. C., and Bernt, K. M. (2017). MLL-Rearranged Leukemias—An Update on Science and Clinical Approaches. Front. Pediatr. 5.
Yahata, T., Shao, W., Endoh, H., Hur, J., Coser, K. R., Sun, H., Ueda, Y., Kato, S., Isselbacher, K. J., Brown, M., et al. (2001). Selective coactivation of estrogen-dependent transcription by CITED1 CBP/p300-binding protein. Genes Dev. 15, 2598-2612.
Yan, J., Enge, M., Whitington, T., Dave, K., Liu, J., Sur, I., Schmierer, B., Jolma, A., Kivioja, T., Taipale, M., et al. (2013). Transcription factor binding in human cells occurs in dense clusters formed around cohesin anchor sites. Cell 154, 801-813.
Yang, F., DeBeaumont, R., Zhou, S., and Nssr, A. M. (2004). The activator-recruited cofactor/Mediator coactivator subunit ARC92 is a functionally important target of the VP16 transcriptional activator. Proc. Natl. Acad. Sci. U.S.A. 101, 2339-2344.
Yang, X., Boehm, J. S., Yang, X., Salehi-Ashtiani, K., Hao, T., Shen, Y., Lubonja, R., Thomas, S. R., Alkan, O., Bhimdi, T., et al. (2011). A public genome-scale lentiviral expression library of human ORFs. Nat. Methods 8, 659-661.
Yu, D., Cattoglio, C., Xue, Y., and Zhou, Q. (2019). A complex between DYRK1A and DCAF7 phosphorylates the C-terminal domain of RNA polymerase II to promote myogenesis. Nucleic Acids Res. 47, 4462-4475.
Zhou, L., Canagarajah, B., Zhao, Y., Baibakov, B., Tokuhiro, K., Maric, D., and Dean, J. (2017). BTBD18 Regulates a Subset of piRNA-Generating Loci through Transcription Elongation in Mice. Dev. Cell 40, 453-466.e5.
24. Kiani S et al. Cas9 gRNA engineering for genome editing, activation and repression. Nature Methods 12, 1051 (2015).
25. Beerli R. R. et al Toward controlling gene expression at will: Specific regulation of the erbB-2/HER-2 promoter by using polydactyl zinc finger proteins constructed from modular building blocks PNAS 95, 14628 (1995).
26. Cong, L., Zhou, R., Kuo, Yc. et al. Comprehensive interrogation of natural TALE DNA-binding modules and transcriptional repressor domains. Nat Commun 3, 968 (2012).
27. Gilbert L A, Larson M H, Morsut L, Liu Z, Brar G A, Torres S E, Stern-Ginossar N, Brandman O, Whitehead E H, Doudna J A, Lim W A, Weissman J S, Qi L S Cell. 2013 Jul. 9. pii: S0092-8674(13)00826-X.
28. Sanson, K. R., Hanna, R. E., Hegde, M. et al. Optimized libraries for CRISPR-Cas9 genetic screens with multiple modalities. Nat Commun 9, 5416 (2018).

1. A heterologous transcriptional activator comprising:
a DNA targeting domain, optionally an enzymatically inactive CRISPR-CAS protein, a zinc finger DNA binding domain, a tet-repressor, or transcriptional activator-like effector (TALE) DNA binding domain; and
an effector domain comprising
at least one transactivation domain (TAD) selected from the TADs listed in Table 2 or Table 6, or a functional variant thereof, optionally Table 6, or
at least two TADs selected from the TADs listed in Table 1 or Table 3, or functional variants thereof, preferably at least one TAD selected from the TADs listed in Table 4, or Table 5 or Table 6, and/or functional variants thereof, wherein the DNA targeting domain and the effector domain are operably linked.

2. The transcriptional activator of claim 1, wherein the effector domain comprises at least three, or at least 4 transactivation domains selected from the TADs listed in Table 1 or Table 3 or functional variants thereof.

3. The transcriptional activator of claim 1 or claim 2, further comprising at least one interaction component.

4. The transcriptional activator of any one of claims 1 to 3, wherein the DNA targeting domain and effector domain are domains of a single polypeptide.

5. The transcriptional activator of claim 3, comprising
a first polypeptide comprising the DNA targeting domain and a first interaction component, and
a second polypeptide comprising an effector domain and a second interaction component,
wherein the first and second interaction components interact under suitable conditions.

6. The transcriptional activator of claim 5, wherein the first and second interaction components form an inducible heterodimer pair which interact under inducing conditions, optionally ABI1 and PYL1.

7. The transcriptional activator of any one of claims 1 to 6, wherein the DNA targeting domain comprises a zinc-finger domain.

8. The transcriptional activator of any one of claims 1 to 7, wherein the effector domain comprises at least one TAD selected from any one of the TADs of SEQ ID NO: 103, 104, 105, 106, 167, and 185, optionally at least one TAD selected from any one of the TADs of SEQ ID NO: 90, 91, 46, 47, 101-106, 110, 116-119, 156, 157, 159, 162, 165, 166, 167 and 172.

9. The transcriptional activator of any one of claims 1 to 8, further comprising one or more nuclear localization signals (NLS), optionally an SV40 NLS.

10. The transcriptional activator of any one of claims 1 to 9, wherein the effector domain comprises an amino acid sequence of SEQ ID NO: 121, 123, 125, 127, 129, 131, 133, 135, 174, 176, 178, 180, or 182, or at least 80%, 85%, 90%, 95% or 99% sequence identity to the TADs therein.

11. An isolated nucleic acid encoding the transcriptional activator of any one of claims 1 to 10 or an effector domain of any one of claims 1 to 10.

12. An expression construct comprising the nucleic acid of claim 11 operably linked to one or more promoters and one or more transcription termination sites.

13. A vector comprising the nucleic acid of claim 11 or the expression construct of claim 12, optionally wherein the vector is an adenoviral or lentiviral vector.

14. A cell comprising the transcriptional activator of any one of claims 1 to 10, the nucleic acid of claim 11, the expression construct of claim 12, or the vector of claim 13.

15. A transcriptional activation system comprising:
a) the heterologous transcriptional activator of any one of claims 1 to 10, wherein the DNA targeting domain comprises a CRISPR-Cas protein and
b) at least one gRNA.

16. The transcriptional activation system of claim 15, wherein the at least one gRNA targets a regulatory element of a gene, optionally the regulatory element is a promoter region, an enhancer region, or a distal regulatory site.

17. A method of activating transcription of a target gene in a cell, the method comprising:
a) introducing into the cell the transcriptional activator of any one of claims 1-10, the nucleic acid of claim 11, the expression construct of claim 12, or the vector of claim 13; and
b) culturing the cell under suitable conditions such that the effector domain activates transcription of the target gene.

18. The method of claim 17, wherein the DNA targeting domain comprises a CRISPR-Cas protein, the method further comprises introducing into the cell at least one gRNA, and culturing the cell under suitable conditions such that the at least one gRNA associates with the CRISPR-Cas protein to guide the transcriptional activator to a CRISPR target site.

19. A screening assay, the assay comprising:
a) introducing into a plurality of cells the transcriptional activator of any one of claims 1 to 10, the one or more nucleic acids of claim 11, the one or more expression constructs of claim 12, or the one or more vectors of claim 13, wherein the DNA targeting domain comprises a CRISPR-Cas protein; and a plurality of gRNAs; or introducing a plurality of gRNAs into a population of cells according to claim 14 wherein the DNA targeting domain comprises a CRISPR-Cas protein;
b) culturing the plurality of cells such that the one or more gRNAs associate with the CRISPR-Cas protein and guides the transcriptional activator to a CRISPR target site such that the effector domain activates transcription of a target gene;
c) optionally treating with an amount of a test drug or toxin;
d) optionally culturing the plurality of cells for a period of time to allow for gRNA dropout or enrichment; and
e) collecting the plurality of cells, or a subset thereof.

20. The assay of claim 19, wherein the method further comprises identifying one or more gRNAs that are over- or under-represented in the plurality of cells or subset thereof.

21. A composition comprising the transcriptional activator of any one of claims 1 to 10, the nucleic acid of claim 11, the expression construct of claim 12, the vector of claim 13, or the cell of claim 14.

22. A kit comprising a vial and the heterologous transcriptional activator of any one of claims 1 to 9, the nucleic acid of claim 11, the expression construct of claim 12, the vector of claim 13, the cell of claim 14, or the composition of claim 21 and optionally one or more of: an inducing agent, a gRNA or a gRNA expression construct.


SEQUENCE LISTING (186 sequences)

SEQ ID NO: 1 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 86 bp, synthetic construct
  source 1..86: mol_type = other DNA
gtttcagagctatgctggaaacagcatagcaagttgaaataaggctagtccgttatcaacttgaaaaagtggcaccgagtcggtgc

SEQ ID NO: 3 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 4 - DNA, 40 bp, synthetic construct
  source 1..40: mol_type = other DNA
gtttcagagctacagcagaaatgctgtagcaagttgaaat

SEQ ID NO: 5 - skipped

SEQ ID NO: 6 - protein, 7 aa, synthetic construct
SGGSGGS

SEQ ID NO: 7 - protein, 4 aa, synthetic construct
SGGS

SEQ ID NO: 8 - protein, 6 aa, synthetic construct
GSGSGS

SEQ ID NO: 9 - protein, 8 aa, synthetic construct
INSRSSGS

SEQ ID NO: 10 - DNA, 21 bp, synthetic construct
  source 1..21: mol_type = other DNA
gtacgttctctatcactgata

SEQ ID NO: 11 - protein, 8 aa, synthetic construct
GSQGSGSM

SEQ ID NO: 12 - protein, 8 aa, synthetic construct
GGSVEREG

SEQ ID NO: 13 - DNA, 24 bp, synthetic construct
  source 1..24: mol_type = other DNA
cgactcactatagggagacccaag

SEQ ID NO: 14 - DNA, 22 bp, synthetic construct
  source 1..22: mol_type = other DNA
attcatcttgcgttggtgctcc

SEQ ID NO: 15 - DNA, 21 bp, synthetic construct
  source 1..21: mol_type = other DNA
cagtgtggtggaattctgcag

SEQ ID NO: 16 - DNA, 20 bp, synthetic construct
  source 1..20: mol_type = other DNA
ccgccactgtgctggatatc

SEQ ID NO: 17 - DNA, 46 bp, synthetic construct
  source 1..46: mol_type = other DNA
ggggacaactttgtacaaaaaagttggcggaggcagggtagcgatg

SEQ ID NO: 18 - DNA, 54 bp, synthetic construct
  source 1..54: mol_type = other DNA
ggggacaactttgtacaagaaagttgggtagccttcgcttccaccacttccacc

SEQ ID NO: 19 - DNA, 42 bp, synthetic construct
  source 1..42: mol_type = other DNA
ggaattctgcagatatcaacaagtttgtacaaaaaagttggc

SEQ ID NO: 20 - DNA, 73 bp, synthetic construct
  source 1..73: mol_type = other DNA
ccagtgtggtggaattctgcagatatcaacaagtttgtacaaaaaagttggcggaagtcagggtagcggaagt

SEQ ID NO: 21 - DNA, 71 bp, synthetic construct
  source 1..71: mol_type = other DNA
ccgccactgtgctggatatcaaccactttgtacaagaaagttgggtagccttcgcgttcaacactacctcc

SEQ ID NO: 22 - protein, 7 aa, synthetic construct
PKKKRKV

SEQ ID NO: 23 - DNA, 24 bp, synthetic construct
  source 1..24: mol_type = other DNA
ggaagtcagggtagcggaagtatg

SEQ ID NO: 24 - DNA, 24 bp, synthetic construct
  source 1..24: mol_type = other DNA
ggaggtagtgttgaacgcgaaggc

SEQ ID NO: 25 - DNA, 501 bp, Homo sapiens
  source 1..501: mol_type = genomic DNA
ttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 26 - DNA, 212 bp, Homo sapiens
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 27 - DNA, 380 bp, Cytomegalovirus
  source 1..380: mol_type = genomic DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 28 - DNA, 204 bp, Cytomegalovirus
  source 1..204: mol_type = genomic DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 29 - protein, 60 aa, Homo sapiens
MGEPAGVAGTMESPFSPGLFHRLDEDWDSALFAELGYFTDTDELQLEAANETYENNFDNL

SEQ ID NO: 30 - protein, 60 aa, Homo sapiens
HRLDEDWDSALFAELGYFTDTDELQLEAANETYENNFDNLDFDLDLVPWESDIWDINNQI

SEQ ID NO: 31 - protein, 60 aa, Homo sapiens
MAELMLLSEIADPTRFFTDNLLSPEDWGLQNSTLYSGLDEVAEEQTQLFRCPEQDVPFDG

SEQ ID NO: 32 - protein, 60 aa, Homo sapiens
NPGPDTQLPSGPAQNPGIDFDIEEFFSASNIQTQTEESELSTMTTEPVLESLDIETQTDF

SEQ ID NO: 33 - protein, 60 aa, Homo sapiens
ETQTMSSGFETLGSLFFTSNETQTAMDDFLLADLAWNTMESQFSSVETQTSAEPHTVSNF

SEQ ID NO: 34 - protein, 60 aa, Homo sapiens
MSRLLHAEEWAEVKELGDHHRQPQPHHLPQPPPPPQPPATLQAREHPVYPPELSLLDSTD

SEQ ID NO: 35 - protein, 60 aa, Homo sapiens
MKMEEMSLSGLDNSKLEAIAQEIYADLVEDSCLGFCFEVHRAVKCGYFFLDDTDPDSMKD

SEQ ID NO: 36 - protein, 60 aa, Homo sapiens
QEIYADLVEDSCLGFCFEVHRAVKCGYFFLDDTDPDSMKDFEIVDQPGLDIFGQVFNQWK

SEQ ID NO: 37 - protein, 60 aa, Homo sapiens
FEIVDQPGLDIFGQVFNQWKSKECVCPNCSRSIAASRFAPHLEKCLGMGRNSSRIANRRI

SEQ ID NO: 38 - protein, 60 aa, Homo sapiens
KEECFRSGVAEAPVGSKAPSIDGKEELDLAEKMDIAVSYTGEELDFETVGDIIAIIEDKV

SEQ ID NO: 39 - protein, 60 aa, Homo sapiens
IDGKEELDLAEKMDIAVSYTGEELDFETVGDIIAIIEDKVDDHPEVLDVAAVEAALSFCE

SEQ ID NO: 40 - protein, 60 aa, Homo sapiens
GEELDFETVGDIIAIIEDKVDDHPEVLDVAAVEAALSFCEENDDPQSLPGPWEHPIQQER

SEQ ID NO: 41 - protein, 60 aa, Homo sapiens
IDCREPYAFDTALLEQPCEAEEYRITSAAATSELEEILDFMLCGSDIEPPIGSLESPGAE

SEQ ID NO: 42 - protein, 60 aa, Homo sapiens
EEYRITSAAATSELEEILDFMLCGSDIEPPIGSLESPGAEGCRTPTYHLTETGKNWIEGE

SEQ ID NO: 43 - protein, 60 aa, Homo sapiens
MSAHMSGLEIMDEDQLIKDVLDKFLNCHEQTYDEEFLNTFTHLSQDLLLLPGEVEQDVST

SEQ ID NO: 44 - protein, 60 aa, Homo sapiens
NHMCGHCQDSPFKEEAWALLMDKSPQKATDADPGSLKQAFDDHNIVETVLDLEEDYNVMT

SEQ ID NO: 45 - protein, 60 aa, Homo sapiens
QDSPFKEEAWALLMDKSPQKATDADPGSLKQAFDDHNIVETVLDLEEDYNVMTSFKYQIE

SEQ ID NO: 46 - protein, 60 aa, Homo sapiens
LQNWDFGAQAGGAESLSPSAGAQSPAIIDSDPVDEEVLMSLVVELGLDRANELPELWLGQ

SEQ ID NO: 47 - protein, 60 aa, Homo sapiens
ESLSPSAGAQSPAIIDSDPVDEEVLMSLVVELGLDRANELPELWLGQNEFDFTADFPSSC

SEQ ID NO: 48 - protein, 60 aa, Homo sapiens
MPASVAHVPAAMLPPNVIDTDFIDEEVLMSLVIEMGLDRIKELPELWLGQNEFDFMTDFV

SEQ ID NO: 49 - protein, 60 aa, Homo sapiens
AMLPPNVIDTDFIDEEVLMSLVIEMGLDRIKELPELWLGQNEFDFMTDFVCKQQPSRVSC

SEQ ID NO: 50 - protein, 60 aa, Homo sapiens
DNIEAPHFPLPGLSPPGDASSCFLESLMGFSEPAAEALDPFIDSQFEDTVPASLMEPVPV

SEQ ID NO: 51 - protein, 60 aa, Homo sapiens
MAAESLPFSFGTLSSWELEAWYEDLQEVLSSDENGGTYVSPPGNEEEESKIFTTLDPASL

SEQ ID NO: 52 - protein, 60 aa, Homo sapiens
WYEDLQEVLSSDENGGTYVSPPGNEEEESKIFTTLDPASLAWLTEEEPEPAEVTSTSQSP

SEQ ID NO: 53 - protein, 60 aa, Homo sapiens
MAITLQPSDLIFEFASNGMDDDIHQLEDPSVFPAVIVEQVPYPDLLHLYSGLELDDVHNG

SEQ ID NO: 54 - protein, 60 aa, Homo sapiens
DDIHQLEDPSVFPAVIVEQVPYPDLLHLYSGLELDDVHNGIITDGTLCMTQDQILEGSFL

SEQ ID NO: 55 - protein, 60 aa, Homo sapiens
PRPQRPAETNAHLPPPRPQRPAETKVPEEIPPEVVQEYVDIMEELLGSHPGDTGEPEGQR

SEQ ID NO: 56 - protein, 60 aa, Homo sapiens
PAETKVPEEIPPEVVQEYVDIMEELLGSHPGDTGEPEGQREKGKVEQPQEEDGITSDPGL

SEQ ID NO: 57 - protein, 60 aa, Homo sapiens
EKGKVEQPQEEDGITSDPGLLSYIDKLCSQEDFVTKVEAVIHPRFLEELLSPDPQMDFLA

SEQ ID NO: 58 - protein, 60 aa, Homo sapiens
LSYIDKLCSQEDFVTKVEAVIHPRFLEELLSPDPQMDFLALSQELEQEEGLTLAQLVEKR

SEQ ID NO: 59 - protein, 60 aa, Homo sapiens
RQPPHSRPCLPTAQACTMSHHPAASHDGAQPLRVLFRRLENGRWSSSLLTAPSFHSPEKP

SEQ ID NO: 60 - protein, 60 aa, Homo sapiens
HPAASHDGAQPLRVLFRRLENGRWSSSLLTAPSFHSPEKPGAFLAQSPHVSEKSEGPCVR

SEQ ID NO: 61 - protein, 60 aa, Homo sapiens
PGSHPRHGAGGRPKPSPAGSRGSPVPAGALQPPEYPWMKEKKAAKKTALLPAAAAAATAA

SEQ ID NO: 62 - protein, 60 aa, Homo sapiens
RGSPVPAGALQPPEYPWMKEKKAAKKTALLPAAAAAATAAATGPACLSHKESLEIADGSG

SEQ ID NO: 63 - protein, 60 aa, Homo sapiens
KKAAKKTALLPAAAAAATAAATGPACLSHKESLEIADGSGGGSRRLRTAYTNTQLLELEK

SEQ ID NO: 64 - protein, 60 aa, Homo sapiens
KNELSDHLDAMDSNLDNLQTMLSSHGFSVDTSALLDLFSPSVTVPDMSLPDLDSSLASIQ

SEQ ID NO: 65 - protein, 60 aa, Homo sapiens
MLSSHGFSVDTSALLDLFSPSVTVPDMSLPDLDSSLASIQELLSPQEPPRPPEAENSSPD

SEQ ID NO: 66 - protein, 60 aa, Homo sapiens
SGKQLVHYTAQPLFLLDPGSVDTGSNDLPVLFELGEGSYFSEGDGFAEDPTISLLTGSEP

SEQ ID NO: 67 - protein, 60 aa, Homo sapiens
AQPLFLLDPGSVDTGSNDLPVLFELGEGSYFSEGDGFAEDPTISLLTGSEPPKAKDPTVS

SEQ ID NO: 68 - protein, 60 aa, Homo sapiens
MTGIAAASFFSNTCRFGGCGLHFPTLADLIEHIEDNHIDTDPRVLEKQELQQPTYVALSY

SEQ ID NO: 69 - protein, 60 aa, Homo sapiens
GPVAWGPWRRAAAPVKGEHFCLPEFPLGDPDDVPRPFQPTLEEIEEFLEENMEPGVKEVP

SEQ ID NO: 70 - protein, 60 aa, Homo sapiens
MDVLPMCSIFQELQIVHETGYFSALPSLEEYWQQTCLELERYLQSEPCYVSASEIKFDSQ

SEQ ID NO: 71 - protein, 59 aa, Homo sapiens
DVLPMCSIFQELQIVHETGYFSALPSLEEYWQQTCLELERYLQSEPCYVSASEIKFDSQ

SEQ ID NO: 72 - protein, 60 aa, Homo sapiens
MDVLASYSIFQELQLVHDTGYFSALPSLEETWQQTCLELERYLQTEPRRISETFGEDLDC

SEQ ID NO: 73 - protein, 60 aa, Homo sapiens
YFSALPSLEETWQQTCLELERYLQTEPRRISETFGEDLDCFLHASPPPCIEESFRRLDPL

SEQ ID NO: 74 - protein, 60 aa, Homo sapiens
SRLTAILLQIKCLAEGGDLNSFEFKSLTDSLNDIKSTIDASNISCFQNNVEIHVAHIQSG

SEQ ID NO: 75 - protein, 60 aa, Homo sapiens
MAHYKAADSKREQFRRYLEKSGVLDTLTKVLVALYEEPEKPNSALDFLKHHLGAATPENP

SEQ ID NO: 76 - protein, 60 aa, Homo sapiens
MDYDSYQHYFYDYDCGEDFYRSTAPSEDIWKKFELVPSPPTSPPWGLGPGAGDPAPGIGP

SEQ ID NO: 77 - protein, 60 aa, Homo sapiens
CSFATTDDFYDDPCFDSPDLRFFEDLDPRLMHVGALLKPEEHSHFPAAVHPAPGAREDEH

SEQ ID NO: 78 - protein, 60 aa, Homo sapiens
LRRMKANARERNRMHGLNAALDNLRKVVPCYSKTQKLSKIETLRLAKNYIWALSEILRSG

SEQ ID NO: 79 - protein, 60 aa, Homo sapiens
RRSRRKKANDRERNRMHNLNSALDALRGVLPTFPDDAKLTKIETLRFAHNYIWALTQTLR

SEQ ID NO: 80 - protein, 60 aa, Homo sapiens
DVPLVPEGLLTPEASPVKQSFFHYSEKEQNEIDRLIQQISQLAQGMDRPFSAEAGTGGLE

SEQ ID NO: 81 - protein, 60 aa, Homo sapiens
PLGGLEPLDSNLSLSGAGPPVLSLDLKPWKCQELDFLADPDNMFLEETPVEDIFMDLSTP

SEQ ID NO: 82 - protein, 60 aa, Homo sapiens
DNMFLEETPVEDIFMDLSTPDPSEEWGSGDPEAEGPGGAPSPCNNLSPEDHSFLEDLATY

SEQ ID NO: 83 - protein, 60 aa, Homo sapiens
DPSEEWGSGDPEAEGPGGAPSPCNNLSPEDHSFLEDLATYETAFETGVSAFPYDGFTDEL

SEQ ID NO: 84 - protein, 60 aa, Homo sapiens
CNNLSPEDHSFLEDLATYETAFETGVSAFPYDGFTDELHQLQSQVQDSFHEDGSGGEPTF

SEQ ID NO: 85 - protein, 60 aa, Homo sapiens
GMALPRLIIRKVDKQTALLDADDPVSQLHKCAFYLKDTERMYLCLSQERIIQFQATPCPK

SEQ ID NO: 86 - protein, 60 aa, Homo sapiens
RRVARPPAAPELGALGSPDLSSLSLAVSRSTDELEIIDEYIKENGFGLDGGQPGPGEGLP

SEQ ID NO: 87 - protein, 60 aa, Homo sapiens
SSLSLAVSRSTDELEIIDEYIKENGFGLDGGQPGPGEGLPRLVSRGAASLSTVTLGPVAP

SEQ ID NO: 88 - protein, 60 aa, Homo sapiens
IDTSMYDNELWAPASEGLKPGPEDGPGKEEAPELDEAELDYLMDVLVGTQALERPPGPGR

SEQ ID NO: 89 - protein, 60 aa, Homo sapiens
SEAGTQKLDGPQESRADDSKLMDSLPGNFEITTSTGFLTDLTLDDILFADIDTSMYDFDP

SEQ ID NO: 90 - protein, 60 aa, Homo sapiens
DPSPQPQSLGLKRKSEWSDESEEELEEELELERAPEPEDTWVVETLCGLKMKLKRKRASS

SEQ ID NO: 91 - protein, 60 aa, Homo sapiens
SEEELEEELELERAPEPEDTWVVETLCGLKMKLKRKRASSVLPEHHEAFNRLLGDPVVQK

SEQ ID NO: 92 - protein, 60 aa, Homo sapiens
QETIQRLLEENDQLIRCIVEYQNKGRGNECVQYQHVLHRNLIYLATIADASPTSTSKAME

SEQ ID NO: 93 - protein, 60 aa, Homo sapiens
PQSDPSVEPPLSQETFSDLWKLLPENNVLSPLPSQAMDDLMLSPDDIEQWFTEDPGPDEA

SEQ ID NO: 94 - protein, 58 aa, Homo sapiens
DALDDFDLDMLGSDALDDFDLDMLGSDALDDFDLDMLGSDALDDFDLDMLGGSVEREG

SEQ ID NO: 95 - protein, 60 aa, Homo sapiens
EKKDKVEKEKSEKETTSKKNSHKKTRPRLKNVDRSSAQHLEVTVGDLTVIITDFKEKTKS

SEQ ID NO: 96 - protein, 60 aa, Homo sapiens
SHKKTRPRLKNVDRSSAQHLEVTVGDLTVIITDFKEKTKSPPASSAASADQHSQSGSSSD

SEQ ID NO: 97 - protein, 60 aa, Homo sapiens
SPAEQHGAQDTELSAGTGNFYLESGGSARTDYRAIQLAKEKKQRGAGSNAGASQSTQRKI

SEQ ID NO: 98 - protein, 60 aa, Homo sapiens
YLESGGSARTDYRAIQLAKEKKQRGAGSNAGASQSTQRKIKEGKMSPPHFHASQNSWLCG

SEQ ID NO: 99 - protein, 60 aa, Homo sapiens
SLVVPSGGRPGPAPAAGVQCGAQGVQVQLVQDDPSGEGVLPSARGPATFLPFLTVDLPVY

SEQ ID NO: 100 - protein, 46 aa, Homo sapiens
SHKKTRPRLKNVDRSSAQHLEVTVGDLTVIITDFKEKTKSPPASSA

SEQ ID NO: 101 - protein, 47 aa, Homo sapiens [sequence omitted]

SEQ ID NO: 102 - protein, 29 aa, Homo sapiens
SDESEEELEEELELERAPEPEDTWVVETL

SEQ ID NO: 103 - protein, 25 aa, Homo sapiens
DSDPVDEEVLMSLVVELGLDRANEL

SEQ ID NO: 104 - protein, 15 aa, Homo sapiens
GFSVDTSALLDLFSP

SEQ ID NO: 105 - protein, 49 aa, Homo sapiens
PTQAGEGTLSEALLQLQFDDEDLGALLGNSTDPAVFTDLASVDNSEFQQ

SEQ ID NO: 106 - protein, 27 aa, Homo sapiens
APGLPNGLLSGDEDFSSIADMDFSALL

SEQ ID NO: 107 - protein, 51 aa, Homo sapiens
AGTGNFYLESGGSARTDYRAIQLAKEKKQRGAGSNAGASQSTQRKIKEGKM

SEQ ID NO: 108 - protein, 40 aa, Homo sapiens
SSLSLAVSRSTDELEIIDEYIKENGFGLDGGQPGPGEGLP

SEQ ID NO: 109 - protein, 40 aa, Homo sapiens
LSYIDKLCSQEDFVTKVEAVIHPRFLEELLSPDPQMDFLA

SEQ ID NO: 110 - protein, 63 aa, Homo sapiens
QDSPFKEEAWALLMDKSPQKATDADPGSLKQAFDDHNIVETVLDLEEDYNVMTSFKYQIEGSS

SEQ ID NO: 111 - protein, 61 aa, Homo sapiens
LGQLSPPPEHPGFDALDQLSQVELLGDMDRNEFDQYLNTPGHPDSATGAMALSGHVPVSQV

SEQ ID NO: 112 - protein, 66 aa, Homo sapiens
DVPKPGSPEPQVSGLAANRSLTEGLVLDTMNDSLSKILLDISFPGLDEDPLGPDNINWSQFIPELQ

SEQ ID NO: 113 - protein, 63 aa, Homo sapiens
HRLDEDWDSALFAELGYFTDTDELQLEAANETYENNFDNLDFDLDLVPWESDIWDINNQIGSS

SEQ ID NO: 114 - protein, 59 aa, Homo sapiens
SAHMSGLEIMDEDQLIKDVLDKFLNCHEQTYDEEFLNTFTHLSQDLLLLPGEVEQDVST

SEQ ID NO: 115 - protein, 40 aa, Homo sapiens
YLESGGSARTDYRAIQLAKEKKQRGAGSNAGASQSTQRKI

SEQ ID NO: 116 - protein, 260 aa, Homo sapiens
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 117 - protein, 119 aa, Homo sapiens
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 118 - protein, 124 aa, Homo sapiens
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 119 - protein, 20 aa, Homo sapiens
MLSSHGFSVDTSALLDLFSP

SEQ ID NO: 120 - DNA, 1611 bp, synthetic construct
  source 1..1611: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..1221: note = RELA
  misc_feature 1222..1239: note = Linker and/or NLS sequence
  misc_feature 1240..1611: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 121 - protein, 537 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 122 - DNA, 1221 bp, synthetic construct
  source 1..1221: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..1221: note = RELA
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 123 - protein, 407 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 124 - DNA, 1611 bp, synthetic construct
  source 1..1611: mol_type = other DNA
  misc_feature 4..375: note = HSF1
  misc_feature 376..426: note = Linker and/or NLS sequence
  misc_feature 427..1206: note = RELA
  misc_feature 1207..1224: note = Linker and/or NLS sequence
  misc_feature 1225..1401: note = SPDYE4
  misc_feature 1402..1422: note = Linker and/or NLS sequence
  misc_feature 1423..1602: note = CITED1
  misc_feature 1603..1611: note = Linker and/or NLS sequence
atgggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcgacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttct

SEQ ID NO: 125 - protein, 537 aa, synthetic construct
MGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGS

SEQ ID NO: 126 - DNA, 876 bp, synthetic construct
  source 1..876: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..798: note = P65
  misc_feature 799..816: note = Linker and/or NLS sequence
  misc_feature 817..876: note = miniHSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcatgctgagcagccacggcttcagcgtggacaccagtgccctgctggacctgttcagcccc

SEQ ID NO: 127 - protein, 292 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSMLSSHGFSVDTSALLDLFSP

SEQ ID NO: 128 - DNA, 1098 bp, synthetic construct
  source 1..1098: mol_type = other DNA
  misc_feature 4..90: note = miniSPDYE4
  misc_feature 91..111: note = Linker and/or NLS sequence
  misc_feature 112..291: note = CITED1
  misc_feature 292..351: note = Linker and/or NLS sequence
  misc_feature 352..708: note = P65
  misc_feature 709..726: note = Linker and/or NLS sequence
  misc_feature 727..1098: note = HSF1
atgagcgacgaaagcgaggaggagttggaggaagagttggaactcgaacgagcgcccgagcctgaagatacgtgggtagtagaaacgctgtctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 129 - protein, 366 aa, synthetic construct
MSDESEEELEEELELERAPEPEDTWVVETLSGGSGGSESLSPSAGAQSPAIIDSDPVDEEVLMSLVVELGLDRANELPELWLGQNEFDFTADFPSSCGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 130 - DNA, 1083 bp, synthetic construct
  source 1..1083: mol_type = other DNA
  misc_feature 4..180: note = SPDYE4
  misc_feature 181..201: note = Linker and/or NLS sequence
  misc_feature 202..276: note = miniCITED1
  misc_feature 277..336: note = Linker and/or NLS sequence
  misc_feature 337..693: note = P65
  misc_feature 694..711: note = Linker and/or NLS sequence
  misc_feature 712..1083: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgatagtgacccggtagacgaggaggtactgatgagtctcgttgttgagttgggattggaccgagcgaacgagttgggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 131 - protein, 361 aa, synthetic construct
MDPSPQPQSLGLKRKSEWSDESEEELEEELELERAPEPEDTWVVETLCGLKMKLKRKRASSGGSGGSDSDPVDEEVLMSLVVELGLDRANELGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 132 - DNA, 912 bp, synthetic construct
  source 1..912: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..522: note = miniP65(C)
  misc_feature 523..540: note = Linker and/or NLS sequence
  misc_feature 541..912: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagcgcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 133 - protein, 304 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 134 - DNA, 978 bp, synthetic construct
  source 1..978: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..588: note = miniP65(N)
  misc_feature 589..606: note = Linker and/or NLS sequence
  misc_feature 607..978: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 135 - protein, 326 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSPTQAGEGTLSEALLQLQFDDEDLGALLGNSTDPAVFTDLASVDNSEFQQGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 136 - protein, 60 aa, Homo sapiens
SASPPSTGQNPSVSVQTSQDWKPTRSLIEHVFVTDVTANLITVTVKESPTSVGFFNLRHY

SEQ ID NO: 137 - skipped

SEQ ID NO: 138 - protein, 65 aa, Homo sapiens
GASSEPEAGDWRPEMSPCSNVVVTDVTSNLLTVTIKEFCNPEDFEKVAAGVAGAAGGGGSIGASK

SEQ ID NO: 139 - protein, 60 aa, Homo sapiens
RPSLIARIPVARILGDPEEESWSPSLTNLEKVVVTDVTSNFLTVTIKESNTDQGFFKEKR

SEQ ID NO: 140 - protein, 72 aa, Homo sapiens
IQSANATTKTSETNHTSRPRLKNVDRSTAQQLAVTVGNVTVIITDFKEKTRSSSTSSSTVTSSAGSEQQNQS

SEQ ID NO: 141 - protein, 17 aa, synthetic construct
INSRSSGSPKKKRKVGS

SEQ ID NO: 142 - protein, 237 aa, Homo sapiens
MASGQARPPFEEESPQPSTTVRSPEVVVDDEVPGPSAPWIDPSPQPQSLGLKRKSEWSDESEEELEEELELERAPEPEDTWVVETLCGLKMKLKRKRASSVLPEHHEAFNRLLGDPVVQKFLAWDKDLRVSDKYLLAMVIAYFSRAGLFSWQYQRIHFFLALYLASDMEEDNQAPKQDIFSFLYGKNYSQRPLFHKLRYQLLCSMRWRTWVSPEEMEEIQAYDPEHWVWARDRTLIS

SEQ ID NO: 143 - protein, 336 aa, Homo sapiens
MQKHYTVAWFLYSAPGVDPSPPCRSLGWKRKREWSDESEEEPEKELAPEPEETWVVETLCGLKMKLKQQRVSPILLEHHKDFNSQLAPGVDPSPPHRSFCWKRKMEWWDKSEESEEEPRKVLAPEPEEIWVAEMLCGLKMKLKRRRVSLVLPEHHEAFNRLLEDPVIKRFLAWDKDLRVSDKYLLAMVIAYFSRAGFPSWQYQRLHFFLALYLANDMEEDDEDSKQNIFHFLYGKNRSRIPLLRKRRFQLYRSMNPRARKNRSHIPLVRKRRFQLRRCMNPRARKNRSQIVLFQKRRFHFFCSMSCRAWVSPEELEEIQAYDPEHWVWARDRARLS

SEQ ID NO: 144 - protein, 208 aa, Homo sapiens
MEWWDKSEESLEEEPRKVLAPEPEEIWVAEMLCGLKMKLKRRRVSLVLPEHHEAFNRLLEDPVIKRFLAWDKGLRVSDKYLLAMVIVYFSRAGLPSWQYQCIHFFLALYLANDMEEDDEDPKQNIFYFLYGKTRSRIPLLRKRRFQLCRCMNPRARKNRSQIVLFQKLRFQFFCSMSCRAWVSPEELEEIQAYDPEHWVWARDRARLS

SEQ ID NO: 145 - protein, 402 aa, Homo sapiens
MDRTETRFRKRGQITGKITTSRQPHPQNEQSPQRSTSGYPLQEVVDDEMLGPSAPGVDPSPPCRSLGWKRKREWSDESEEEPEKELAPEPEETWVVEMLCGLKMKLKQQRVSPILPEHHKDFNSQLAPGVDPSPPHRSFCWKRKMEWWDESEESLEEEPRKVLAPEPEEIWVAEMLCGLKMKLKRRRVSLVLPEHHEAFNRLLEDPVIKRFLAWDKDLRVSDKYLLAMVIAYFSRAGFPSWQYQRIHFFLALYLANDMEEDDEDSKQNIFHFLYRKNRSRIPLLRKPWFQLGHSMNPRARKNRSRIPLLRKRRFQLYRSTNPRARKNRSRIPLLRKRRFQLYRSMNSRARKNRSQIVLFQKRRFHFFCSMSCRAWVSPEELEEIQAYDPEHWVWARDRAHLS

SEQ ID NO: 146 - protein, 293 aa, Homo sapiens
MLWAIPELGSPCPISISYEMSDSQDPTTSPVVTTQVELGGCSRQGGGNGFLRFRQHQEVQAFLSLLEDSFVQEFLSKDPCFQISDKYLLAMVLVYFQRAHLKLSEYTHSSLFLALYLANDMEEDLEGPKCEIFPWALGKDWCLRVGKFLHQRDKLWARMGFRAVVSRQCCEEVMAKEPFHWAWTRDRRPHHGGVQRVCPQVPVRLPRGPGLSPPHCSPCGLPQHCSSHLLKPVSSKCPSLTSECHRPPSQNYLSRVKNAWGGDFLIVLPPQMQLEPGTYSLRIFPKPPARPGH

SEQ ID NO: 147 - protein, 33 aa, Homo sapiens
RPRLKNVDRSSAQHLEVTVGDLTVIITDFKEKT

SEQ ID NO: 148 - protein, 33 aa, Homo sapiens
RPRLKNVDRSTAQQLAVTVGNVTVIITDFKEKT

SEQ ID NO: 149 - protein, 34 aa, Homo sapiens
QDWKPTRSLIEHVFVTDVTANLITVTVKESPTSV

SEQ ID NO: 150 - protein, 34 aa, Homo sapiens
GDWRPEMSPCSNVVVTDVTSNLLTVTIKEFCNPE

SEQ ID NO: 151 - protein, 34 aa, Homo sapiens
ESWSPSLTNLEKVVVTDVTSNFLTVTIKESNTDQ

SEQ ID NO: 152 - protein, 30 aa, Homo sapiens
SEFKPFFGNIIITDVTANCLTVTFKEYVTV

SEQ ID NO: 153 - protein, 33 aa, Homo sapiens
PPWTPALPSSEVTVTDITANSITVTFREAQAAE

SEQ ID NO: 154 - protein, 60 aa, Homo sapiens
VRSPEVVVDDEVPGPSAPWIDPSPQPQSLGLKRKSEWSDESEEELEEELELERAPEPEDT

SEQ ID NO: 155 - protein, 60 aa, Homo sapiens
WVVETLCGLKMKLKRKRASSVLPEHHEAFNRLLGDPVVQKFLAWDKDLRVSDKYLLAMVI

SEQ ID NO: 156 - protein, 56 aa, Homo sapiens
SFGTLSSWELEAWYEDLQEVLSSDENGGTYVSPPGNEEEESKIFTTLDPASLAWLT

SEQ ID NO: 157 - protein, 29 aa, Homo sapiens
ERAPEPEDTWVVETLCGLKMKLKRKRASS

SEQ ID NO: 158 - protein, 44 aa, Homo sapiens
TGNFYLESGGSARTDYRAIQLAKEKKQRGAGSNAGASQSTQRKI

SEQ ID NO: 159 - protein, 45 aa, Homo sapiens
GFSVDTSALLDLFSPSVTVPDMSLPDLDSSLASIQELLSPQEPPR

SEQ ID NO: 160 - protein, 40 aa, Homo sapiens
LMDSLPGNFEITTSTGFLTDLTLDDILFADIDTSMYDFDP

SEQ ID NO: 161 - protein, 74 aa, Homo sapiens
MAEEFVTLKDVGMDFTLGDWEQLGLEQGDTFWDTALDNCQDLFLLDPPRPNLTSHPDGSEDLEPLAGGSPEATS

SEQ ID NO: 162 - protein, 44 aa, Homo sapiens
AIIDSDPVDEEVLMSLVVELGLDRANELPELWLGQNEFDFTADF

SEQ ID NO: 163 - protein, 84 aa, Homo sapiens
MQQVTTLQLPPSRVSPFKDMILCFSEEDWSLLDPAQTGFYGEFIIGEDYGVSMPPNDLAAQPDLSQGEENEPRVPELQDLQGKE

SEQ ID NO: 164 - protein, 44 aa, Homo sapiens
VIDTDFIDEEVLMSLVIEMGLDRIKELPELWLGQNEFDFMTDFV

SEQ ID NO: 165 - protein, 54 aa, Homo sapiens
PTQAGEGTLSEALLQLQFDDEDLGALLGNSTDPAVFTDLASVDNSEFQQLLNQG

SEQ ID NO: 166 - protein, 33 aa, Homo sapiens
ATDADPGSLKQAFDDHNIVETVLDLEEDYNVMT

SEQ ID NO: 167 - protein, 20 aa, Homo sapiens
DDHNIVETVLDLEEDYNVMT

SEQ ID NO: 168 - protein, 26 aa, Homo sapiens
DADPGSLKQAFDDHNIVETVLDLEED

SEQ ID NO: 169 - protein, 28 aa, Homo sapiens
DSPFKEEAWALLMDKSPQKATDADPGSL

SEQ ID NO: 170 - protein, 33 aa, Homo sapiens
DLQEVLSSDENGGTYVSPPGNEEEESKIFTTLD

SEQ ID NO: 171 - protein, 21 aa, Homo sapiens
LSSWELEAWYEDLQEVLSSDE

SEQ ID NO: 172 - protein, 43 aa, Homo sapiens
ATDADPGSLKQAFDDHNIVETVLDLEEDYNVMTSFKYQIEGSS

SEQ ID NO: 173 - DNA, 1098 bp, synthetic construct
  source 1..1098: mol_type = other DNA
  misc_feature 44..180: note = SPDYE4
  misc_feature 181..192: note = Linker and/or NLS sequence
  misc_feature 193..291: note = C3orf62.2
  misc_feature 292..351: note = Linker and/or NLS sequence
  misc_feature 352..708: note = P_AD
  misc_feature 709..726: note = Linker and/or NLS sequence
  misc_feature 727..1098: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcagcaacagacgccgacccgggtagccttaaacaggccttcgacgatcataatatcgtggagaccgtgctggacctggaggaagattacaatgtaatgactggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 174 - protein, 366 aa, synthetic construct
MDPSPQPQSLGLKRKSEWSDESEEELEEELELERAPEPEDTWVVETLCGLKMKLKRKRASSGGSATDADPGSLKQAFDDHNIVETVLDLEEDYNVMTGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 175 - DNA, 1059 bp, synthetic construct
  source 1..1059: mol_type = other DNA
  misc_feature 4..180: note = SPDYE4
  misc_feature 181..192: note = Linker and/or NLS sequence
  misc_feature 193..252: note = C3orf62.3
  misc_feature 253..312: note = Linker and/or NLS sequence
  misc_feature 313..669: note = P_AD
  misc_feature 670..687: note = Linker and/or NLS sequence
  misc_feature 688..1059: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcagatgaccacaatattgtggagaccgtgctcgatcttgaagaggattacaatgtaatgacgggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 176 - protein, 353 aa, synthetic construct
MDPSPQPQSLGLKRKSEWSDESEEELEEELELERAPEPEDTWVVETLCGLKMKLKRKRASSGGSDDHNIVETVLDLEEDYNVMTGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 177 - DNA, 1128 bp, synthetic construct
  source 1..1128: mol_type = other DNA
  misc_feature 4..180: note = SPDYE4
  misc_feature 181..192: note = Linker and/or NLS sequence
  misc_feature 193..321: note = C3orf62_MT
  misc_feature 322..381: note = Linker and/or NLS sequence
  misc_feature 382..738: note = P_AD
  misc_feature 739..756: note = Linker and/or NLS sequence
  misc_feature 757..1128: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcagccaccgatgccgacccagggtccttgaagcaggcgttcgatgatcataatatcgtagaaacggtcctcgatctggaggaagactacaatgttatgacttcatttaagtatcagatagagggttcctctggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 178 - protein, 376 aa, synthetic construct
MDPSPQPQSLGLKRKSEWSDESEEELEEELELERAPEPEDTWVVETLCGLKMKLKRKRASSGGSATDADPGSLKQAFDDHNIVETVLDLEEDYNVMTSFKYQIEGSSGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 179 - DNA, 1176 bp, synthetic construct
  source 1..1176: mol_type = other DNA
  misc_feature 4..180: note = SPDYE4
  misc_feature 181..201: note = Linker and/or NLS sequence
  misc_feature 202..369: note = DDIT3_MT
  misc_feature 370..429: note = Linker and/or NLS sequence
  misc_feature 430..786: note = P_AD
  misc_feature 787..804: note = Linker and/or NLS sequence
  misc_feature 805..1176: note = HSF1
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcagctttggtaccctgtcatcctgggagctggaagcgtggtatgaggacttgcaggaggttttgagttcagacgaaaacggtggtacctatgtatctcctccgggaaatgaagaagaagagtccaaaatctttaccacccttgacccagcttctttggcgtggctcaccggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 180 - protein, 392 aa, synthetic construct
MDPSPQPQSLGLKRKSEWSDESEEELEEELELERAPEPEDTWVVETLCGLKMKLKRKRASSGGSGGSSFGTLSSWELEAWYEDLQEVLSSDENGGTYVSPPGNEEEESKIFTTLDPASLAWLTGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 181 - DNA, 951 bp, synthetic construct
  source 1..951: mol_type = other DNA
  misc_feature 4..180: note = SPDYE4
  misc_feature 181..201: note = Linker and/or NLS sequence
  misc_feature 202..381: note = CITED1
  misc_feature 382..441: note = Linker and/or NLS sequence
  misc_feature 442..798: note = P_AD
  misc_feature 799..816: note = Linker and/or NLS sequence
  misc_feature 817..951: note = HSF1_MT
atggacccttcccctcaaccccaaagcctgggtcttaaaagaaaatccgaatggagcgacgaaagcgaggaggagttggaggaagagttggaactcgaacgagcgcccgagcctgaagatacgtgggtagtagaaacgctgtgcggattgaagatgaaattgaaacggaagagagcgagttctggcgggtcaggaggaagcgaatccctcagcccctccgctggcgcacagagccctgctattatagatagtgacccggtagacgaggaggtactgatgagtctcgttgttgagttgggattggaccgagcgaacgagttgcccgaactgtggctggggcagaacgagtttgattttactgcagactttccgtctagctgcggaggttctattaactctagaagttccggatctccgaaaaagaaacgcaaagttggtagccctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagcggctttagtgttgatacctcagctctgcttgacttgttctcacccagtgttaccgttcctgatatgagtctgcctgatctcgactcaagtttggcgtcaattcaggagctgctgagccctcaggagcctcctaga

SEQ ID NO: 182 - protein, 317 aa, synthetic construct
MDPSPQPQSLGLKRKSEWSDESEEELEEELELERAPEPEDTWVVETLCGLKMKLKRKRASSGGSGGSESLSPSAGAQSPAIIDSDPVDEEVLMSLVVELGLDRANELPELWLGQNEFDFTADFPSSCGGSINSRSSGSPKKKRKVGSPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSGFSVDTSALLDLFSPSVTVPDMSLPDLDSSLASIQELLSPQEPPR

SEQ ID NO: 183 - DNA, 666 bp, synthetic construct
  source 1..666: mol_type = other DNA
atggcggaagagtttgtaacgctcaaggatgtaggaatggattttacattgggtgactgggaacagcttggtttggaacagggtgatacgttctgggatactgcattggataactgccaagatctgttcctgctggatcccccgaggccaaatcttacctcacatcccgatggcagtgaggatcttgaacctctggcaggaggaagtcccgaagcaactagtatggcagaagaatttgtgacattgaaggacgtgggaatggactttacattgggggactgggaacaattgggtcttgagcagggtgatacattttgggacacagctctcgacaattgccaagatcttttcttgctggatccaccccgccctaacctgacatcacatccagacggtagcgaggatttggaacccctggccggaggtagtcctgaggcaacctccatggcggaggaatttgtgactctgaaggatgtagggatggacttcacgctcggagattgggagcagctcggattggagcaaggtgacacattctgggacaccgcacttgacaactgccaagatctttttcttctcgacccaccgcggccgaacttgactagccaccccgatggctctgaagatcttgaaccactcgccgggggatcacctgaagccacgagc

SEQ ID NO: 184 - protein, 222 aa, synthetic construct
MAEEFVTLKDVGMDFTLGDWEQLGLEQGDTFWDTALDNCQDLFLLDPPRPNLTSHPDGSEDLEPLAGGSPEATSMAEEFVTLKDVGMDFTLGDWEQLGLEQGDTFWDTALDNCQDLFLLDPPRPNLTSHPDGSEDLEPLAGGSPEATSMAEEFVTLKDVGMDFTLGDWEQLGLEQGDTFWDTALDNCQDLFLLDPPRPNLTSHPDGSEDLEPLAGGSPEATS

SEQ ID NO: 185 - protein, 15 aa, Homo sapiens
ERAPEPEDTWVVETL

SEQ ID NO: 186 - protein, 206 aa, Escherichia coli
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESG